(19) United States
(12) Patent Application Publication
(10) Pub. No.: US 2025/0213547 A1
(43) Pub. Date: Jul. 3, 2025

(54) METHODS OF TREATING NEUROLOGICAL DISEASES

(71) Applicant: TAKEDA PHARMACEUTICAL COMPANY LIMITED, Osaka (JP)

(72) Inventors: Mahnaz ASGHARNEJAD, Cambridge, MA (US); Wei YIN, Cambridge, MA (US); Naga Venkatesha Murthy PATHI JAGANNATHAM, Lexington, MA (US)

(21) Appl. No.: 18/848,069

(22) PCT Filed: Mar. 17, 2023
(86) PCT No.: PCT/IB2023/052659

§ 371 Date: Sep. 17, 2024

Related U.S. Application Data
(60) Provisional application No. 63/321,409, filed on Mar. 18, 2022.

(51) Int. Cl. A61K 31/4545; A61K 9/00; A61P 25/08

(52) U.S. Cl. CPC A61K 31/4545; A61K 9/0053; A61P 25/08

(57) ABSTRACT

The invention describes the pharmacokinetic and pharmacodynamic properties of soticlestat and defines significant covariates thereof. The invention provides safe and effective dosages and dose regimens of soticlestat for treating neurological disease.

### TECHNICAL FIELD

The present disclosure relates to the methods for administering soticlestat or a pharmaceutically acceptable salt thereof to a human patient at a safe, tolerable, and therapeutic dose for the treatment or prevention of a neurological disease caused or exacerbated by increased levels of 24-hydroxycholersterol. The present invention discloses soticlestat pharmacokinetic and pharmacodynamic parameters, and the safe and therapeutic dosages providing an effective exposure level, as well as the covariates affecting soticlestat bioavailability and CH24H enzyme inhibition.

### BACKGROUND

The enzyme cholesterol 24-hydroxylase (CH24H), also known as cytochrome P450 46A1 (CYP461) is mostly expressed in neurons where it catalyzes the hydroxylation of cholesterol to the oxysterol 24-hydroxycholesterol (24HC) in the endoplasmic reticulum. 24HC has rapid efflux from neurons into the extracellular space and moves through the blood brain barrier into systemic circulation for hepatic elimination. However, high levels of CH24H activity can increase 24HC levels in the extracellular space where it can act as a positive allosteric modulator of N-methyl-D-aspartate (NMDA) receptor activity in neurons. 24HC may also stimulate CH24H expression in astrocytes, which would deplete the cholesterol in lipid rafts required for glutamate uptake and decrease astrocytic cholesterol export to neurons. Decreased glutamate uptake could markedly increase extracellular glutamate leading to excitotoxicity in neurons.

High levels of 24HC can increased tonic neuronal hyperactivity resulting increased activity of NMDA receptor, depolarization, and excitotoxicity. 24HC-mediated neuronal hyperactivity could contributed to developmental and epileptic encephalopathies (DEE) and neurodegenerations. DEE is characterized by multiple seizure types, and developmental delay or regression, and affects approximately 100,000-200,000 people in the USA. DEE includes a number of orphan syndromes, including Lennox-Gastaut syndrome (LGS), with an estimated prevalence of 1-5 individual per 10,000 worldwide, and Dravet syndrome (DS), with an estimated prevalence of 1 individual per 15,700 people in the USA. Many DEE patients experience daily seizures resulting in a lower quality of life and significant morbidity. The seizures of most patients are refractory to current anti-seizure treatments and therefore there is a significant need for an effective therapy.

Increased NMDA receptor activation may produce excitotoxicity in other neurodegenerations, such as Huntington's disease and Parkinson's disease. Epileptiform seizure activity is present in about half of Alzheimer's disease patients and is associated with faster cognitive decline. Accordingly, postmortem CH24H levels are significantly higher in glia cells from Alzheimer's disease patient's brains than from cognitive controls. Yet, plasma 24HC is significantly decreased in Alzheimer's disease patients compared to 24HC levels from cognitive controls. This indicates that 24HC may accumulate in brains of Alzheimer's disease patients and could cause induce NMDA-mediated epileptiform seizure activity and excitotoxicity mediated neuron death, contributing to Alzheimer's disease etiology. Moreover, 24HC may decrease cholesterol synthesis in glia cells. Decreased cholesterol synthesis and increased cholesterol hydroxylation in astrocytes would decrease cholesterol transport to neurons, which would deplete their cholesterol rich lipid rafts, resulting in reduced signal transduction and cognitive deficits.

In summary, decreasing CH24H activity by pharmacological intervention could have therapeutic value for patients with epilepsy and neurodegenerations. The drug soticlestat selectively binds to CH24H inhibiting enzyme activity. Thus, soticlestat may have significant therapeutic potential for treating disorders caused by 24HC-mediated neuronal hyperactivity, such as DEE and other neurodegenerations.

### SUMMARY

Increased levels of 24-hydroxycholesterol (24HC) is associated with neurological disease. The drug soticlestat selectively binds cholesterol 24-hydroxylase (CH24H), the enzyme that metabolizes cholesterol to 24-hydroxycholesterol (24HC), and robustly inhibits enzyme activity. Decreasing 24HC levels has the potential to treat neurological diseases such as developmental epileptic encephalopathy or epileptic type disease, such as epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like. The present invention defines soticlestat pharmacokinetic and pharmacodynamic parameters and identifies significant covariates and their effect size on soticlestat exposure and biological response. The present disclosure describes the safe, tolerable, and effective dosage range of soticlestat for treating neurological disease associated with increased 24-hydroxycholesterol (24HC).

In one aspect, disclosed herein is a method of treating a neurological disease in a human in need thereof, the method comprising administering to the human a therapeutically effective amount of a dosage form comprising soticlestat or a pharmaceutically acceptable salt thereof, wherein the human reaches a soticlestat steady-state plasma area under the curve (AUC₂₄) from about 449±219 to about 6118±4841 h×ng/ml following administration.

In another aspect, disclosed herein is a method of treating a neurological disease in a human in need thereof, the method comprising administering to the human a therapeutically effective amount of a dosage form comprising soticlestat or a pharmaceutically acceptable salt thereof, wherein the human reaches a maximum blood plasma concentration (Cₘₐₓ) of from about 204±177 to about 2063±821 ng/ml following administration.

In another aspect, disclosed herein is a method of treating a neurological disease in a human in need thereof, the method comprising administering to the human a therapeutically effective amount of soticlestat or a pharmaceutically acceptable salt thereof, wherein the human reaches a reduction from baseline in plasma 24HC of at least about 50% following administration.

In another aspect, disclosed herein is a method of treating a neurological disease in a human in need thereof, the method comprising administering to the human a therapeutically effective amount of a solution comprising soticlestat or a pharmaceutically acceptable salt thereof, wherein the human reaches a soticlestat steady-state plasma area under the curve (AUC₂₄) from about 350 to about 2900 h×ng/ml following administration.

In another aspect, disclosed herein is a method of treating a neurological disease in a human in need thereof, the method comprising administering to the human a therapeutically effective amount of a solution comprising soticlestat or a pharmaceutically acceptable salt thereof, wherein the human reaches a maximum blood plasma concentration (Cₘₐₓ) of from about 430 to about 2980 ng/ml.

In another aspect, disclosed herein is a method of reducing plasma 24HC levels in a human, the method comprising administering to the human a therapeutically effective amount of soticlestat or a pharmaceutically acceptable salt thereof in a solution or a solid dosage form, wherein the human reaches a reduction from baseline in plasma 24HC of at least about 50% following administration.

In another aspect, disclosed herein is a method of reducing seizure frequency in a human, the method comprising administering to the human a therapeutically effective amount of soticlestat or a pharmaceutically acceptable salt thereof in a solution or a solid dosage form, wherein the human achieves a reduction in seizure frequency of at least about 30% following administration.

In some embodiments, the therapeutically effective amount is a total daily dosage of from about 80 to about 600 mg of soticlestat or a pharmaceutically acceptable salt thereof. In some embodiments, the total daily dosage is about 200 mg, about 400 mg, or about 600 mg. In some embodiments, the total daily dose is 80 mg, 120 mg, 160 mg, 200 mg, 240 mg, or 400 mg for a human, wherein the human is a pediatric patient. In some embodiments, the total daily dosage is administered twice daily. In some embodiments, the human is a pediatric patient.

In another aspect, disclosed herein is a method of minimizing the risk of one or more adverse events associated with soticlestat administration, the method comprising administering to a human having a neurological disease a total daily dosage of from about 80 mg to about 600 mg of soticlestat or a pharmaceutically acceptable salt thereof, wherein the soticlestat or a pharmaceutically acceptable salt thereof is administered in a solution or a solid dosage form.

In some embodiments, soticlestat or a pharmaceutically acceptable salt thereof is administered once daily. In some embodiments, soticlestat or a pharmaceutically acceptable salt thereof is administered twice daily.

In some embodiments, the total daily dosage is about 100 mg once daily, about 200 mg once daily, about 300 mg once daily, or about 400 mg once daily. In some embodiments, the total daily dosage is about 200 mg, about 400 mg, or about 600 mg, wherein the total daily dosage is administered in two administrations per day.

In some embodiments, the human reaches an AUC₂₄ from about 800 to about 2290 h×ng/ml. In some embodiments, the human reaches an AUC₂₄ from about 800 to about 2900 h×ng/ml. In some embodiments, the time to reach maximum blood plasma concentration (Tₘₐₓ) is about 2 hours or less after oral administration. In some embodiments, the time to reach maximum blood plasma concentration (Tₘₐₓ) is about 0.5 to 2 hours after oral administration. In some embodiments, the terminal elimination half-life is from about 1.7 to about 7.1 hours.

In some embodiments, the total daily dosage is from about 200 to about 600 mg, wherein the human is administered soticlestat for at least 7 days, and wherein the AUC₁₂ on day 7 is within 160% of the AUC₁₂ on day 1.

In some embodiments, the total daily dosage is from about 200 to about 600 mg, wherein the human is administered soticlestat for at least 7 days, and wherein the Cₘₐₓ on day 7 is within 220% of the Cₘₐₓ on day 1.

In some embodiments, the therapeutically effective amount is a total daily dosage of about 200 mg, administered twice daily, and wherein the human reaches a soticlestat steady-state plasma area under the curve (AUC₂₄) of about 449 ng×h/ml±219 ng×h/ml.

In some embodiments, the therapeutically effective amount is a total daily dosage of about 400 mg, administered twice daily, and wherein the human reaches a soticlestat steady-state plasma area under the curve (AUC₂₄) of about 767 ng×h/ml±235 ng×h/ml.

In some embodiments, the therapeutically effective amount is a total daily dosage of about 600 mg, administered twice daily, and wherein the human reaches a soticlestat steady-state plasma area under the curve (AUC₂₄) of about 4036 ng×h/ml±1018 ng×h/ml.

In some embodiments, the therapeutically effective amount is a total daily dosage of about 200 mg, administered twice daily, and wherein the human reaches a maximum blood plasma concentration (Cₘₐₓ) of about 204 ng/ml±177 ng/ml.

In some embodiments, the therapeutically effective amount is a total daily dosage of about 400 mg, administered twice daily, and wherein the human reaches a maximum blood plasma concentration (Cₘₐₓ) of about 253 ng/ml±109 ng/ml.

In some embodiments, the therapeutically effective amount is a total daily dosage of about 600 mg, administered twice daily, and wherein the human reaches a maximum blood plasma concentration (Cₘₐₓ) of about 2063 ng/ml±831 ng/ml.

In some embodiments, the neurological disease is epilepsy, rare epilepsy, developmental epileptic encephalopathy, epileptic encephalopathy, Dravet syndrome, Lennox-Gastaut syndrome, focal onset seizures, major motor seizures, major motor drop seizures, focal seizures with secondary generalization, infantile spasms, primary generalized tonic-clonic seizures, partial onset seizures with our without secondary generalization, simple partial seizures, complex partial seizures, simple absence seizures, complex absence seizures, Dup15q syndrome, CDKL5 deficiency disorder, migraine, or complex regional pain syndrome.

In some embodiments, the soticlestat is a free base of crystalline Form II. In some embodiments, the crystalline Form II is characterized by (i) an x-ray powder diffraction (XRPD) pattern comprising three or more, four or more, five or more, six or more, seven or more, eight or more, nine or more, or ten 2θ values selected from 9.4, 10.8, 13.0, 15.3, 17.2, 18.2, 18.8, 19.4, 20.1, and 21.6±0.2°2θ, and/or (ii) an XPRD pattern substantially in accordance with FIG. 2.

In some embodiments, the soticlestat is a free base of crystalline Form I. In some embodiments, the crystalline Form I is characterized by (i) an x-ray powder diffraction (XRPD) pattern comprising three or more, four or more, five or more, six or more, seven or more, eight or more, nine or more, or ten 2θ values selected from 9.0, 9.6, 11.3, 12.3, 14.1, 15.7, 17.4, 20.9, 21.6, and 22.0±0.2°2θ, and/or (ii) an XPRD pattern substantially in accordance with FIG. 1.

In some embodiments, the soticlestat is a crystalline 3.0 hydrate of soticlestat. In some embodiments, the crystalline 3.0 hydrate is characterized by (i) an x-ray powder diffraction (XRPD) pattern comprising three or more, four or more, five or more, six or more, seven or more, eight or more, nine or more, or ten 2θ values selected from 8.8, 9.3, 12.4, 14.8, 16.9, 20.5, 20.9, 21.9, 22.3, and 24.5±0.2°2θ, and/or (ii) an XPRD pattern substantially in accordance with FIG. 3.

In some embodiments, the solid dosage form is a tablet.

In some embodiments, soticlestat or a pharmaceutically acceptable salt thereof is administered daily for at least fourteen days, optionally wherein the human is in a fasted state at the time of administration.

In yet another aspect, disclosed herein is a method of treating a neurological disease in a human in need thereof, the method comprising administering to the human about 100 mg once daily of soticlestat or a pharmaceutically acceptable salt thereof.

In yet another aspect, disclosed herein is a method of treating a neurological disease in a human in need thereof, the method comprising administering to the human about 200 mg once daily of soticlestat or a pharmaceutically acceptable salt thereof.

In yet another aspect, disclosed herein is a method of treating a neurological disease in a human in need thereof, the method comprising orally administering to the human about 300 mg once daily of soticlestat or a pharmaceutically acceptable salt thereof.

In yet another aspect, disclosed herein is a method of treating a neurological disease in a human in need thereof, the method comprising administering to the human about 400 mg once daily of soticlestat or a pharmaceutically acceptable salt thereof.

In yet another aspect, disclosed herein is a method of treating a neurological disease in a human in need thereof, the method comprising administering to the human a total daily dosage of about 200 mg of soticlestat or a pharmaceutically acceptable salt thereof, wherein the soticlestat or a pharmaceutically acceptable salt thereof is administered twice daily.

In yet another aspect, disclosed herein is a method of treating a neurological disease in a human in need thereof, the method comprising administering to the human a total daily dosage of about 400 mg of soticlestat or a pharmaceutically acceptable salt thereof, wherein the soticlestat or a pharmaceutically acceptable salt thereof is administered twice daily.

In yet another aspect, disclosed herein is a method of treating a neurological disease in a human in need thereof, the method comprising administering to the human a total daily dosage of about 600 mg of soticlestat or a pharmaceutically acceptable salt thereof, wherein the soticlestat or a pharmaceutically acceptable salt thereof is administered twice daily.

In yet another aspect, disclosed herein is a method of treating a neurological disease in a human in need thereof, the method comprising administering to the human a total daily dosage of about 80 mg, 120 mg, 160 mg, 200 mg, 240 mg, or 400 mg of soticlestat or a pharmaceutically acceptable salt thereof, wherein the soticlestat or pharmaceutically acceptable salt thereof is administered twice daily.

In some embodiments, the human is a pediatric patient.

In some embodiments, the soticlestat or pharmaceutically acceptable salt thereof is administered orally.

In some embodiments, the soticlestat or pharmaceutically acceptable salt thereof is administered by a gastrostomy or jejunostomy tube delivery.

In some embodiments, the soticlestat or pharmaceutically acceptable salt thereof is administered in an oral solution.

In some embodiments, the soticlestat or pharmaceutically acceptable salt thereof is administered in a solid oral dosage form. In some embodiments, the solid oral dosage form is a tablet.

In some embodiments, the neurological disease is epilepsy, rare epilepsy, developmental epileptic encephalopathy, epileptic encephalopathy, Dravet syndrome, Lennox-Gastaut syndrome, focal onset seizures, major motor seizures, major motor drop seizures, focal seizures with secondary generalization, infantile spasms, primary generalized tonic-clonic seizures, partial onset seizures with our without secondary generalization, simple partial seizures, complex partial seizures, simple absence seizures, complex absence seizures, Dup15q syndrome, CDKL5 deficiency disorder, migraine, or complex regional pain syndrome. In some embodiments, the neurological disease is developmental epileptic encephalopathy or epileptic type disease. In some embodiments, the neurological disease is Dravet syndrome or Lennox-Gastaut syndrome.

In some embodiments, the soticlestat is a free base of crystalline Form II. In some embodiments, the crystalline Form II is characterized by (i) an x-ray powder diffraction (XRPD) pattern comprising three or more, four or more, five or more, six or more, seven or more, eight or more, nine or more, or ten 2θ values selected from 9.4, 10.8, 13.0, 15.3, 17.2, 18.2, 18.8, 19.4, 20.1, and 21.6±0.2°2θ, and/or (ii) an XPRD pattern substantially in accordance with FIG. 2.

In some embodiments, the soticlestat is a free base of crystalline Form I. In some embodiments, the crystalline Form I is characterized by (i) an x-ray powder diffraction (XRPD) pattern comprising three or more, four or more, five or more, six or more, seven or more, eight or more, nine or more, or ten 2θ values selected from 9.0, 9.6, 11.3, 12.3, 14.1, 15.7, 17.4, 20.9, 21.6, and 22.0±0.2°2θ, and/or (ii) an XPRD pattern substantially in accordance with FIG. 1.

In some embodiments, the soticlestat is a crystalline 3.0 hydrate of soticlestat. In some embodiments, the crystalline 3.0 hydrate is characterized by (i) an x-ray powder diffraction (XRPD) pattern comprising three or more, four or more, five or more, six or more, seven or more, eight or more, nine or more, or ten 2θ values selected from 8.8, 9.3, 12.4, 14.8, 16.9, 20.5, 20.9, 21.9, 22.3, and 24.5±0.2°2θ, and/or (ii) an XPRD pattern substantially in accordance with FIG. 3.

In some embodiments, the dosage form is orally administered.

In some embodiments, the dosage form is administered by gastronomy or jejunostomy tube.

In some embodiments, soticlestat is co-administered with an antiseizure medication. In some embodiments, the co-administration is sequential. In some embodiments, the co-administration is at the same time. In some embodiments, the antiseizure medication is valproic acid, clobazam, stiripentol, topiramate, clonazepam (klonipin), levetiracetam, zonisamide, gelbatolm, epidiolex, fintepla, zonisamide, pyridoxine, ethosuximide, diazepam, brivaracetam, prednisone, prednisolone methylprednisolone, fycompa, phenobarbital, valproate, or lamotrigine, rufinamide.

In one aspect, the present disclosure provides a method of treating a neurological disease in a human in need thereof, the method comprising administering to the human a therapeutically effective amount of a dosage form comprising soticlestat or a pharmaceutically acceptable salt thereof, wherein the human reaches a soticlestat steady-state plasma area under the curve (AUC₂₄) from about 460 to about 2290 h×ng/ml following administration.

In another aspect, the present disclosure provides method of treating a neurological disease in a human in need thereof, the method comprising administering to the human a therapeutically effective amount of a dosage form comprising soticlestat or a pharmaceutically acceptable salt thereof, wherein the human reaches a maximum blood plasma concentration (Cₘₐₓ) of from about 220 to about 1025 ng/ml.

In another aspect, the present disclosure provides a method of treating a neurological disease in a human in need thereof, the method comprising administering to the human a therapeutically effective amount of soticlestat or a pharmaceutically acceptable salt thereof, wherein the human reaches a reduction from baseline in plasma 24HC of at least about 40% following administration.

In another aspect, the present disclosure provides a method of treating a neurological disease in a human in need thereof, the method comprising administering to the human a therapeutically effective amount of a solution comprising soticlestat or a pharmaceutically acceptable salt thereof, wherein the human reaches a soticlestat steady-state plasma area under the curve (AUC₂₄) from about 350 to about 2900 h×ng/ml following administration.

In another aspect, the present disclosure provides a method of treating a neurological disease in a human in need thereof, the method comprising administering to the human a therapeutically effective amount of a solution comprising soticlestat or a pharmaceutically acceptable salt thereof, wherein the human reaches a maximum blood plasma concentration (Cₘₐₓ) of from about 430 to about 2980 ng/ml.

In another aspect, the present disclosure provides a method of reducing plasma 24HC levels in a human, the method comprising administering to the human a therapeutically effective amount of soticlestat or a pharmaceutically acceptable salt thereof in a solution or a solid dosage form, wherein the human reaches a reduction from baseline in plasma 24HC of at least about 40% following administration.

In another aspect, the present disclosure provides a method of reducing seizure frequency in a human, the method comprising administering to the human a therapeutically effective amount of soticlestat or a pharmaceutically acceptable salt thereof in a solution or a solid dosage form, wherein the human achieves a reduction in seizure frequency of at least about 30% following administration.

In another aspect, the present disclosure provides a method of minimizing the risk of one or more adverse events associated with soticlestat administration, the method comprising administering to a human having a neurological disease a total daily dosage of from about 100 mg to about 400 mg of soticlestat or a pharmaceutically acceptable salt thereof, wherein the soticlestat or a pharmaceutically acceptable salt thereof is administered in a solution or a solid dosage form.

These and other aspects may be characterized by one or more optional embodiments. In some embodiments, the therapeutically effective amount is a total daily dosage of from about 100 to about 600 mg of soticlestat or a pharmaceutically acceptable salt thereof. In some embodiments, the total daily dosage is from about 100 to about 400 mg. In some embodiments, soticlestat or a pharmaceutically acceptable salt thereof is administered once daily. In some embodiments, soticlestat or a pharmaceutically acceptable salt thereof is administered twice daily. In some embodiments, the total daily dosage is about 100 mg once daily, about 200 mg once daily, about 300 mg once daily, or about 400 mg once daily. In some embodiments, the total daily dosage is about 200 mg, about 400 mg, or about 600 mg, and the total daily dosage is administered in two administrations per day. In some embodiments, the human reaches an AUC₂₄ from about 800 to about 2290 h×ng/ml. In some embodiments, the human reaches an AUC₂₄ from about 800 to about 2900 h×ng/ml.

In some embodiments, the time to reach maximum blood plasma concentration (Tₘₐₓ) is about 0.6 hours or less after oral administration. In some embodiments, the Tₘₐₓ is about 0.5 hours or less after oral administration. In some embodiments, the terminal elimination half-life is from about 3 to about 5 hours. In some embodiments, the total daily dosage is from about 100 to about 400 mg, and the human is administered soticlestat for at least 14 days, and the AUC₂₄ on day 14 is within 10% of the AUC₂₄ on day 1. In some embodiments, the total daily dosage is from about 100 to about 400 mg, and the human is administered soticlestat for at least 14 days, and the Cₘₐₓ on day 14 is within 10% of the Cₘₐₓ on day 1.

In some embodiments, the therapeutically effective amount is a total daily dosage of about 200 mg, administered twice daily, and the human reaches a soticlestat steady-state plasma area under the curve (AUC₂₄) of about 563 ng×h/ml±100 ng×h/ml. In some embodiments, the therapeutically effective amount is a total daily dosage of about 400 mg, administered twice daily, and the human reaches a soticlestat steady-state plasma area under the curve (AUC₂₄) of about 1437 ng×h/ml±100 ng×h/ml. In some embodiments, the therapeutically effective amount is a total daily dosage of about 600 mg, administered twice daily, and the human reaches a soticlestat steady-state plasma area under the curve (AUC₂₄) of about 2188 ng×h/ml±100 ng×h/ml. In some embodiments, the therapeutically effective amount is a total daily dosage of about 200 mg, administered twice daily, and the human reaches a maximum blood plasma concentration (Cₘₐₓ) of about 270 ng/ml±50 ng/ml. In some embodiments, the therapeutically effective amount is a total daily dosage of about 400 mg, administered twice daily, and the human reaches a maximum blood plasma concentration (Cₘₐₓ) of about 640 ng/ml±50 ng/ml. In some embodiments, the therapeutically effective amount is a total daily dosage of about 600 mg, administered twice daily, and the human reaches a maximum blood plasma concentration (Cₘₐₓ) of about 975 ng/ml±50 ng/ml. In some embodiments, the therapeutically effective amount is a total daily dosage of about 100 mg, administered once daily, and the human reaches a soticlestat steady-state plasma area under the curve (AUC₂₄) of about 458 ng×h/ml±100 ng×h/ml. In some embodiments, the therapeutically effective amount is a total daily dosage of about 300 mg, administered once daily, and the human reaches a soticlestat steady-state plasma area under the curve (AUC₂₄) of about 2690 ng×h/ml±100 ng×h/ml. In some embodiments, the therapeutically effective amount is a total daily dosage of about 400 mg, administered once daily, and the human reaches a soticlestat steady-state plasma area under the curve (AUC₂₄) of about 2800 ng×h/ml±100 ng×h/ml. In some embodiments, the therapeutically effective amount is a total daily dosage of about 100 mg, administered once daily, and the human reaches a maximum blood plasma concentration (Cₘₐₓ) of about 481 ng/ml±50 ng/ml. In some embodiments, the therapeutically effective amount is a total daily dosage of about 300 mg, administered once daily, and the human reaches a maximum blood plasma concentration (Cₘₐₓ) of about 3100 ng/ml±50 ng/ml. In some embodiments, the therapeutically effective amount is a total daily dosage of about 400 mg, administered once daily, and the human reaches a maximum blood plasma concentration (Cₘₐₓ) of about 2930 ng/ml±50 ng/ml.

In some embodiments, the method further comprises determining the total daily dosage to be administered to the human based on one or more covariates of the human selected from age, body weight, body mass index, antiepileptic comedication, baseline plasma alpha-1 acid glycoprotein level, plasma eGFR level, formulation type, Asian origin, or any combination thereof. In some embodiments, one of the one or more covariates for determining the total daily dosage comprises baseline plasma alpha-1 acid glycoprotein level. In some embodiments, one of the one or more covariates for determining the total daily dosage comprises antiepileptic comedication. In some embodiments, one of the one or more covariates for determining the total daily dosage comprises plasma eGFR level. In some embodiments, one of the one or more covariates for determining the total daily dosage comprises Asian ethnic origin. In some embodiments, one of the one or more covariates for determining the total daily dosage comprises body mass index or body weight.

In some embodiments, the neurological disease is epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome. In some embodiments, the neurological disease is developmental epileptic encephalopathy or epileptic type disease. In some embodiments, the neurological disease is Dravet syndrome or Lennox-Gastaut syndrome.

In some embodiments, the soticlestat is a free base of crystalline Form II. In some embodiments, the crystalline Form II is characterized by (i) an x-ray powder diffraction (XRPD) pattern comprising three or more, four or more, five or more, six or more, seven or more, eight or more, nine or more, or ten 2θ values selected from 9.4, 10.8, 13.0, 15.3, 17.2, 18.2, 18.8, 19.4, 20.1, and 21.6±0.2°2θ, and/or (ii) an XPRD pattern substantially in accordance with FIG. 2.

In some embodiments, the soticlestat is a free base of crystalline Form I. In some embodiments, the crystalline Form I is characterized by (i) an x-ray powder diffraction (XRPD) pattern comprising three or more, four or more, five or more, six or more, seven or more, eight or more, nine or more, or ten 2θ values selected from 9.0, 9.6, 11.3, 12.3, 14.1, 15.7, 17.4, 20.9, 21.6, and 22.0±0.2°2θ, and/or (ii) an XPRD pattern substantially in accordance with FIG. 1.

In some embodiments, the soticlestat is a crystalline 3.0 hydrate of soticlestat. In some embodiments, the crystalline 3.0 hydrate is characterized by (i) an x-ray powder diffraction (XRPD) pattern comprising three or more, four or more, five or more, six or more, seven or more, eight or more, nine or more, or ten 2θ values selected from 8.8, 9.3, 12.4, 14.8, 16.9, 20.5, 20.9, 21.9, 22.3, and 24.5±0.2°2θ, and/or (ii) an XPRD pattern substantially in accordance with FIG. 3.

In some embodiments, the solid dosage form is a tablet.

In some embodiments, soticlestat or a pharmaceutically acceptable salt thereof is administered daily for at least fourteen days.

In some embodiments, the human is in a fasted state at the time of administration.

In some embodiments, the dosage form is orally administered. In some embodiments, the dosage form is administered by gastronomy tube.

In some embodiments, soticlestat is co-administered with an antiseizure medication. In some embodiments, the co-administration is sequential. In some embodiments, the co-administration is at the same time. In some embodiments, the antiseizure medication is stiripentol, clobazam, valproic acid, lamotrigine, fenfluramine, or cannabidiol.

In another aspect, the present disclosure provides a method of treating a neurological disease in a human in need thereof, the method comprising orally administering to the human about 100 mg once daily of soticlestat or a pharmaceutically acceptable salt thereof.

In another aspect, the present disclosure provides a method of treating a neurological disease in a human in need thereof, the method comprising orally administering to the human about 200 mg once daily of soticlestat or a pharmaceutically acceptable salt thereof.

In another aspect, the present disclosure a method of treating a neurological disease in a human in need thereof, the method comprising orally administering to the human about 300 mg once daily of soticlestat or a pharmaceutically acceptable salt thereof.

In another aspect, the present disclosure provides a method of treating a neurological disease in a human in need thereof, the method comprising orally administering to the human about 400 mg once daily of soticlestat or a pharmaceutically acceptable salt thereof.

In another aspect, the present disclosure provides a method of treating a neurological disease in a human in need thereof, the method comprising orally administering to the human a total daily dosage of about 200 mg of soticlestat or a pharmaceutically acceptable salt thereof, wherein the soticlestat or a pharmaceutically acceptable salt thereof is administered twice daily.

In another aspect, the present disclosure provides a method of treating a neurological disease in a human in need thereof, the method comprising orally administering to the human a total daily dosage of about 400 mg of soticlestat or a pharmaceutically acceptable salt thereof, wherein the soticlestat or a pharmaceutically acceptable salt thereof is administered twice daily.

In another aspect, the present disclosure a method of treating a neurological disease in a human in need thereof, the method comprising orally administering to the human a total daily dosage of about 600 mg of soticlestat or a pharmaceutically acceptable salt thereof, wherein the soticlestat or a pharmaceutically acceptable salt thereof is administered twice daily.

These and other aspects may be characterized by one or more optional embodiments. In some embodiments, the soticlestat is administered in an oral solution. In some embodiments, the soticlestat is administered in a solid oral dosage form. In some embodiments, the solid oral dosage form is a tablet.

In some embodiments, the neurological disease is epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome. In some embodiments, the neurological disease is developmental epileptic encephalopathy or epileptic type disease. In some embodiments, the neurological disease is Dravet syndrome or Lennox-Gastaut syndrome.

In some embodiments, the soticlestat is a soticlestat free base of crystalline Form II. In some embodiments, the crystalline Form II is characterized by (i) an x-ray powder diffraction (XRPD) pattern comprising three or more, four or more, five or more, six or more, seven or more, eight or more, nine or more, or ten 2θ values selected from 9.4, 10.8, 13.0, 15.3, 17.2, 18.2, 18.8, 19.4, 20.1, and 21.6±0.2°2θ, and/or (ii) an XPRD pattern substantially in accordance with FIG. 2.

In some embodiments, the soticlestat is a soticlestat free base of crystalline Form I. In some embodiments, the crystalline Form I is characterized by (i) an x-ray powder diffraction (XRPD) pattern comprising three or more, four or more, five or more, six or more, seven or more, eight or more, nine or more, or ten 2θ values selected from 9.0, 9.6, 11.3, 12.3, 14.1, 15.7, 17.4, 20.9, 21.6, and 22.0±0.2°2θ, and/or (ii) an XPRD pattern substantially in accordance with FIG. 1.

In some embodiments, the soticlestat is a crystalline 3.0 hydrate of soticlestat. In some embodiments, the crystalline 3.0 hydrate is characterized by (i) an x-ray powder diffraction (XRPD) pattern comprising three or more, four or more, five or more, six or more, seven or more, eight or more, nine or more, or ten 2θ values selected from 8.8, 9.3, 12.4, 14.8, 16.9, 20.5, 20.9, 21.9, 22.3, and 24.5±0.2°2θ, and/or (ii) an XPRD pattern substantially in accordance with FIG. 3.

In some embodiments, the dosage form is orally administered. In some embodiments, the dosage form is administered by gastronomy tube.

In some embodiments, soticlestat is co-administered with an antiseizure medication. In some embodiments, the co-administration is sequential. In some embodiments, the co-administration is at the same time. In some embodiments, the antiseizure medication is stiripentol, clobazam, valproic acid, lamotrigine, fenfluramine, or cannabidiol.

### BRIEF DESCRIPTION OF THE DRAWINGS

FIG. 1 shows the X-ray powder diffraction pattern of the soticlestat polymorph Form I crystal, (4-benzyl-4-hydroxypiperidin-1-yl) (2,4′-bipyridin-3-yl) methanone.

FIG. 2 shows the X-ray powder diffraction pattern of the soticlestat polymorph Form II crystal, (4-benzyl-4-hydroxypiperidin-1-yl) (2,4′-bipyridin-3-yl) methanone.

FIG. 3 shows the X-ray powder diffraction pattern of the soticlestat polymorph 3.0 hydrate crystal, (4-benzyl-4-hydroxypiperidin-1-yl) (2,4′-bipyridin-3-yl) methanone.

FIG. 4A shows the mean linear plasma concentration-time profiles of soticlestat from 0 to 96 hours after administration of a single oral dose.

FIG. 4B shows the mean log-linear plasma concentration-time profiles of soticlestat from 0 to 96 hours after administration of a single oral dose.

FIG. 4C shows the mean linear plasma concentration-time profiles of soticlestat from 0 to 24 hours after administration of a single oral dose.

FIG. 4D shows the mean log-linear plasma concentration-time profile of soticlestat from 0 to 24 hours after administration of a single oral dose.

FIG. 5A shows a graph of the mean linear plasma concentration-time profile after a single oral dose of 300 mg soticlestat administered in oral solution or tablet formulation in the fasting condition from 0 to 48 hours.

FIG. 5B shows a graph of the mean log-linear plasma concentration-time profile after a single oral dose of 300 mg soticlestat administered in oral solution or tablet formulation in the fasting condition from 0 to 48 hours.

FIG. 5C shows a graph of the mean linear plasma concentration-time profile after a single oral dose of 300 mg soticlestat administered in the tablet formulation in the fasted and fed condition from 0 to 48 hours.

FIG. 5D shows a graph of the mean log-linear plasma concentration-time profile after a single oral dose of 300 mg soticlestat administered in the tablet formulation in the fasted and fed condition from 0 to 48 hours.

FIG. 6A shows a graph of the mean plasma concentration-time profile of 24HC for 24 hours before soticlestat dosing (baseline).

FIG. 6B shows a graph of the mean plasma concentration-time profile of 24HC for 24 hours after soticlestat dosing.

FIG. 6C shows a graph of the mean plasma concentration-time profile of 24HC for 96 hours before soticlestat dosing.

FIG. 7A shows a graph of the mean plasma soticlestat concentration after a single oral dose of soticlestat on day 1 from 0 to 24 hours.

FIG. 7B shows a graph of the mean plasma M-I concentration after a single oral dose of soticlestat on day 1 from 0 to 24 hours.

FIG. 7C shows a graph of the mean plasma soticlestat concentration after multiple oral doses of soticlestat as measured on day 14 from 0 to 24 hours.

FIG. 7D shows a graph of the mean plasma M-I concentration after multiple oral doses of soticlestat as measured on day 14 from 0 to 24 hours.

FIG. 8 shows a graph of the mean time-matched percent change from baseline in trough plasma 24HC concentrations with daily soticlestat dosing at different concentration over a 14-day period. Note that cohorts receiving 300 mg BID and 600 mg/day dosing discontinued dosing after day 7.

FIG. 9A shows a schematic depicting the study paradigm including part A, a randomization, double-blind placebo controlled study for 30 days, and part B, an open-label extension study for 60 days, followed by a 30 follow-up period.

FIG. 9B shows a schematic depicting the patient screening and subject disposition in the trial. The superscript “a” by screen failure (n=8) refers to the 5 patients who did not meet the screening criteria plus the 3 patients who withdrew. The superscript “b” by withdrawals (n=2) refers to 1 patient who experienced weakness and another patient who experienced difficulty walking and worsening lethargy. The superscript “c” by withdrawals (n=2) in period two refers to the 2 patients who experienced seizure clusters.

FIG. 10 shows the mean and standard deviation of 24HC levels in plasma at baseline, during the study (Part A and Part B), and 36 days after the last dose.

FIG. 11A shows the median seizure frequency in part B of the study for all completers during the maintenance phase separated into two intervals including days 31-42 and days 42-85. The data is calculated for a 28-day average.

FIG. 11B shows the median seizure frequency in part B of the study, excluding the three patients taking perampanel, during the maintenance phase (days 42-85). The data is calculated for a 28-day average.

FIG. 12 shows a schematic of the 2-compartment linear population PK model including dose, and distribution into plasma and tissue.

FIG. 13 shows a scatterplot of the population and individual predicted versus the observed soticlestat plasma concentration.

FIG. 14 shows a scatterplot of soticlestat population and individual conditionally weighted residuals in relation to time after first dose.

FIG. 15 shows a scatterplot of soticlestat population and individual conditionally weighted residuals at time after most recent dose.

FIG. 16 shows a scatterplot of the population predicted conditionally weighted residuals at population predicted soticlestat concentrations.

FIG. 17 shows a visual predictive check plot of the predicted-corrected residuals from all PK concentration data up to 48 hours.

FIG. 18 shows a visual predictive check plot of the predicted-corrected observation residuals from different study groups up to 24 hours after dose.

FIG. 19 shows a visual predictive check plot of the predicted-corrected observation residuals from different study groups up to 6 hours after dose.

FIG. 20 shows a visual predictive check plot of the predicted-corrected observation residuals at different dose concentrations up to 1-hour post dose.

FIG. 21 shows a tornado plot of the change in AUC₂₄ with simulated changes in the covariate reference value for dose, A1-AGLP, body weight, and patient.

FIG. 22 shows a tornado plot of the change in Cₘₐₓ with simulated changes in the covariate reference value for dose, A1-AGLP, strong inducer, body weight, BMI, formulation, and patient.

FIG. 23 shows a tornado plot of the change in Ctrough with simulated changes in the covariate reference value for dose, A1-AGLP, body weight, patient, Asian origin, eGFR, and strong inducer.

FIG. 24 shows a graph of the effect of changes in body weight on steady state AUC₂₄.

FIG. 25 shows a graph of the effect of different A1-AGLP concentrations on steady state AUC₂₄.

FIG. 26 shows a schematic of the PK/OE/24HC model structure displaying observed and unobserved data, the two compartment linear PK model, together with the PD model depicting the brain soticlestat concentration, enzyme occupancy, and 24HC turnover.

FIG. 27 shows a scatterplot of the predicted population and individual 24HC concentration versus observed plasma 24HC concentrations.

FIG. 28 shows 24HC individual weighted residual scatterplots at top left and normalized predication error top right plotted against time after first dose. The bottom two scatterplots show estimated individual and population conditional weight residuals plotted against time after first dose. The centerline is the regression of the mean and the lines above and below are the regression of the + and—the absolute residuals.

FIG. 29 shows 24HC individual weighted residual scatterplots at top left and normalized predication error top right plotted against the population predicted concentration (ng/mL). The bottom two scatterplots show estimated individual and population conditional weight residuals plotted against the population predicted concentration (ng/ml). The centerline is the regression line of the mean and the lines above and below are the regression of the + and—the absolute residuals.

FIG. 30 shows a tornado plot of the percent change in 24HC concentration from baseline in relation to a simulated covariate change from reference value (X-axis). The Y-axis shows the four covariates, dose (mg), A1-AGLP (mg/dL), weight (kg), and patient.

FIG. 31 shows a tornado plot of the percent change in enzyme occupancy in relation to a simulated covariate change from reference value (X-axis). The Y-axis shows the four covariates, dose (mg), A1-AGLP (mg/dL), weight (kg), and patient.

FIG. 32 shows the differential scanning calorimetry (DSC) trace and thermogravimetric analysis (TGA) thermogram for the soticlestat polymorph Form I crystal.

FIG. 33 shows the differential scanning calorimetry (DSC) trace for the soticlestat polymorph Form II crystal.

FIG. 34 shows the differential scanning calorimetry (DSC) trace and thermogravimetric analysis (TGA) thermogram for the soticlestat polymorph 3.0 hydrate crystal.

### DETAILED DESCRIPTION

The compound soticlestat potently and selectively inhibits cholesterol 24-hydroxylase (CH24H) enzyme activity and is bioavailable through oral administration. Soticlestat is a promising drug for treating neurological disease related to upregulated CH24H activity, which results in excess 24HC production and/or cholesterol depletion. To realize the therapeutic potential of soticlestat it is necessary to determine the pharmacokinetic (PK) and pharmacodynamic (PD) parameters of soticlestat, so that safe, tolerable, and clinically effective dosage regimens are developed. The PK parameters define how soticlestat interacts with the body to affect bioavailability, while the PD parameters define how soticlestat interacts with the CH24H to produce the biological effect, in this case decreased HC24 levels from decreased CH24H activity. Soticlestat bioavailability (F) is the amount of the extravascular soticlestat dose that enters the blood and indicates exposure. The PK parameters that influence soticlestat relative bioavailability are absorption, distribution, metabolism, and elimination, and these values can vary for different dosages, regimens, and formulations. PK/PD parameters that influence soticlestat are the 24HC baseline value (BL), the first-order elimination rate (KOUT), the soticlestat concentration resulting in maximum inhibitory activity (Iₘₐₓ), and the soticlestat concentration resulting in 50% enzyme inhibition (IC50).

Factors that can influence soticlestat bioavailability and its biological effect are covariates such as age, weight, body mass index, race, and concomitant anti-epileptic drug usage, the drug's affinity to serum proteins, and liver and kidney function. For instance, a subject's body weight often affects a drug's absorption rate constant through increased volume of distribution. A covariate that explains between subject variability can predict individual differences in bioavailability and the biological response. The relationships between covariates and drug exposure or biological response can be characterized by population modeling. Subject covariates are incorporated into the model to accurately define the PK and PK/PD parameters and sources of variability in a population. Discovering the significant covariates and their effect size can aid in selecting the appropriate soticlestat dosage for individuals that maximizes the therapeutic effect, while minimizing adverse effects.

The invention described herein defines soticlestat PK and PK/PD parameters, and the covariates with significant effects on between subject variability. In addition, the invention defines the safe, tolerable, and effective soticlestat exposure (AUC) for treating neurological disease related to elevated levels of 24HC. PK and PK/PD data was derived from multiple clinical trials using healthy subjects (examples 2-8) and patients with epileptic encephalopathies (examples 9-11). The cumulative data from these trials was used in stepwise non-linear mix effects modeling analyses to define the population PK and PK/PD parameters, the exposure parameters, and the significant covariates and their effect size (examples 12 and 13). Soticlestat efficacy for reducing seizures was studied in pediatric patients with DEE (example 14).

The data from the clinical trials described herein were obtained from subjects administered the soticlestat polymorph Form II. Thus, in some embodiments of the present invention, a composition containing soticlestat Form II is administered to treat neurological disease. In other embodiments, a composition containing soticlestat Form I or 3.0 hydrate is administered to treat neurological disease. The methods for producing the soticlestat polymorphs and their melting point and X-ray power diffraction (XRD) characterization are described in example 1.

The first-in-human clinical trial administered a single-rising dose and included 48 healthy men and women aged 19-55 with a body mass index between 18 kg/m2 and 30 kg/m2. Subjects were excluded from this study if they were pregnant or had a history of seizures or convulsions, gastrointestinal disease, or drug abuse, including alcohol and nicotine, or a positive drug finding in the urine screening, or any uncontrolled illness, a positive result for hepatitis B antigen or hepatitis C antibody, or history of immunodeficiency. Subjects were administered either oral solution of 300 mg soticlestat or the placebo after a 10 hour fast. Blood samples of 4 ml were collected 30 minutes before dosing with an oral solution of soticlestat on day 1 and at 0.17, 0.25, 0.33, 0.5, 1, 1.5, 2, 3, 4, 6, 8, 10, 12, 16, 24, 36, 48, and 72 hours after dosing. The trial results indicated a safe dosage range and demonstrated soticlestat CH24H inhibitory effect based on decreased plasma 24HC levels. Examples 2-5 herein further describes the trial protocol and the results, including data on the bioavailability of solution versus tablet (example 3), fed versus fasting patients (example 4), and PD data (example 5). The data obtained from this trial informed on the safe dosage range for future trials, and was included in the first-step of the modeling analysis described in example 12.

The trial also compared the bioavailability of soticlestat administered in solution versus tablet formulation, and the effect of food on bioavailability. This study included nine patients in a three-sequence regimen altering fed state and formulation. Soticlestat Cₘₐₓ in fasted subjects was 36.9% lower in the patients administered the tablet formulation and the time to Cₘₐₓ (Tₘₐₓ) was 0.53 hour compared to 0.35 hour for the oral solution. However, soticlestat exposure was similar between solution and tablet formulations, with AUC_{∞} in the tablet only 15.8% lower than the solution in fasted subjects, 7.315 ng×h/mL compared to 7.144 ng×h/mL, respectively.

The second trial was a phase I, randomized, double-blind, placebo-controlled study, with soticlestat administered by oral solution at multiple escalating doses in healthy subjects. The criteria for subject inclusion and exclusion are similar to those of the first trial described above but with the added screening of female subjects for pregnancy. Five cohorts with eight subjects each, including two placebos, were randomized to receive soticlestat by oral solution for 14 days. After a cohort finished their dosage regimen, a decision was made whether to escalate the dosage for the next cohort. Cohorts 1, 2, and 3 received 100, 300, and 400 mg once daily (QD), respectively, and cohorts 4 and 5 received 300 mg twice daily (BID) or 600 mg QD. The regimen for cohorts 4 and 5 were discontinued after subjects began experiencing adverse neurological and psychiatric events. Example 6 describes the PK protocol and the results.

The blood sampling protocol included obtaining a 4-ml blood sample for PK analysis 30 minutes pre-dose, and at 10, 15, 20, and 30 minutes and 1, 1.5, 2, 3, 4, 6, 8, 10, 12, 16, and 24 hours post-dose on days 1 and 14. Cohort 3, receiving 400 mg QD, had additional blood samples taken on day 7 at 15 and 30 minutes, and 1, 2, 4, and 8 hours post-dose. The mean Cₘₐₓ for doses 100, 300, and 400 mg were 481, 3100, and 2930 ng/ml, respectively. The median time for Cₘₐₓ (Tₘₐₓ) across the dose range varied between 0.33-0.5 hours. The mean terminal half-life (t1/2z) for soticlestat did not change from day 1 to day 14, ranging from 3.49 to 4.83 hours. The 24 hour plasma area under the concentration-time curve (AUC₂₄) for doses 100, 300, and 400 mg at day 14 were 458, 2690, and 2800 ng×h/mL, respectively. The concentration time profiles were similar from day 1 through day 14. However, mean Cₘₐₓ and AUC₂₄ increased disproportionately to soticlestat dose, with a 4-fold dose increase resulting in a 6.08-fold and 6.12 fold increase in Cₘₐₓ and AUC₂₄, respectively.

In one embodiment of the present invention, soticlestat oral solution is administered to a patient at a daily dosage between 100 and 400 mg to treat a neurological disease. In certain embodiments, soticlestat oral solution is administered to a patient at a daily dosage between 100 and 400 mg as a prophylaxis or treatment to reduce symptoms of developmental epileptic encephalopathies, epilepsy, and the like. In another embodiment, soticlestat oral solution is administered to a patient at a daily dosage between 100 and 400 mg as a prophylaxis or treatment for neurodegenerative diseases. In certain embodiments, soticlestat oral solution is administered once or twice daily. In certain embodiments, soticlestat oral solution is administered in the fasting condition.

In certain embodiments, a soticlestat in oral solution formulation is administered to a patient in need of treatment for a neurological disease by selecting a dose that reaches a target steady-state soticlestat plasma AUC₂₄ from about 350 to about 2900 ng×h/mL, e.g., from about 458 to about 2800 ng×h/mL. In certain embodiments, soticlestat oral solution is administered to a patient at a dose wherein the target steady-state soticlestat plasma AUC₂₄ reaches from about 350 to about 2900 ng×h/mL (e.g., from about 458 to about 2800 ng×h/mL) as a prophylaxis or treatment to reduce symptoms of developmental epileptic encephalopathies, epilepsy, and the like. In certain embodiments, soticlestat oral solution is administered to a patient at a dose that reaches a target steady-state soticlestat plasma AUC₂₄ from about 350 to about 2900 ng×h/ml (e.g., from about 458 to about 2800 ng×h/mL) as a prophylaxis or treatment for neurodegenerative diseases (e.g., epilepsy, rare epilepsy, developmental epileptic encephalopathy, Dravet syndrome, Lennox-Gastaut syndrome, Dup15q syndrome, CDKL5 deficiency disorder, migraine, complex regional pain syndrome, Alzheimer's disease, mild cognitive disorder, Huntington's disease, Parkinson's disease, amyotrophic lateral sclerosis, traumatic brain injury, cerebral infarction, glaucoma, multiple sclerosis, and the like). In some embodiments, soticlestat is administered in a solid form, which may include a tablet or capsule formulation.

In certain embodiments, soticlestat oral solution is administered to patient in need of treatment for a neurological disease, wherein following administration, the patient's plasma soticlestat reaches a Cₘₐₓ from about 430 to about 2980 ng/ml, e.g., from about 481 to about 2930 ng/mL. In certain embodiments, soticlestat oral solution is administered to patient as a prophylaxis or treatment to reduce symptoms of developmental epileptic encephalopathies, epilepsy, and the like, wherein a patient's plasma soticlestat Cₘₐₓ reaches from about 430 to about 2980 ng/ml (e.g., from about 481 to about 2930 ng/ml). In certain embodiments, soticlestat oral solution is administered to patient as a prophylaxis or treatment for neurodegenerative diseases (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like), wherein the patient reaches a plasma soticlestat Cₘₐₓ from about 430 to about 2980 ng/ml (e.g., from about 481 to about 2930 ng/mL).

An 8 mL blood sample was collected to obtain plasma 24HC levels for PD analysis on day 1 and day 14 at 30 minutes pre-dose and at 30 minutes, and 1, 2, 4, 8, 12, 16, and 24 hours post-dose. The mean 24-hour area under the effect curve (AUEC₂₄), presented as the percent change in 24HC from baseline, for doses 100, 300, and 400 mg were −46.8, −61.9, and −62.7, respectively. Soticlestat treatment at 100-400 mg produced a robust decrease in 24HC levels that plateaued at day 7. Example 7 below further describes the study's PD protocol and trial results, including safety (example 8).

In some embodiments, soticlestat oral solution is administered to a patient to treat neurological disease at a dosage that reaches a target percent decrease in 24HC from baseline of at least 40%, at least 50%, at least 60%, at least 70%, or at least 80%. In some embodiments, soticlestat oral solution is administered to a patient as a prophylaxis or treatment to reduce symptoms of developmental epileptic encephalopathies, epilepsy, wherein the effective dosage produces a target percent decrease in 24HC from baseline of at least 40%, at least 50%, at least 60%, at least 70%, or at least 80%. In some embodiments, soticlestat oral solution is administered to a patient as a prophylaxis or treatment for neurodegenerative diseases (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like), wherein the dose produces a target percent decrease in 24HC from baseline of at least 40%, at least 50%, at least 60%, at least 70%, or at least 80%.

The third clinical trial was a randomized, double-blind, placebo controlled study. The trial was divided into two parts, Part A and Part B. In Part A, 18 adult patients with developmental epileptic encephalopathies (DEE) received soticlestat (n=14) or placebo (n=4) for 30 days. After the completion of Part A, 16 patients continued in Part B with open-label treatment for 60 additional days. Patients were included in the trial if they were 18-65 years of age with an established diagnosis of DEE, such as Lennox-Gastaut syndrome, Dravet syndrome, or tuberous sclerosis complex. For enrollment, patients were required to have had at least two bilateral motor seizures per month in the three months prior. In addition, during the 4-week baseline period prior to dosing, patients were required to have incurred at least one bilateral motor seizure. Exclusion criteria included abnormal ECG, degenerative eye disease, or admission to emergency care for treatment of status epilepticus that required mechanical ventilation.

In Part A, patient dosages were titrated, with soticlestat administered at 100 mg BID for days 1-10, 200 mg BID for days 11-20, and if tolerated, 300 mg BID for days 21-30. Soticlestat was administered in tablet formulation during fasting condition. Two patients could not tolerate 100 mg soticlestat and were withdrawn from the study. Blood samples were obtained before the first morning dose, and at 1, 3, and 5 hours post-dose. On days 11 and 21, blood samples were taken before the morning dose and 1 hour post-dose. In Part B, patients were administered 200 mg BID for days 31-40, followed by 300 mg BID for days 41-85. On days 31, 41, and 85, blood samples were taken before the morning dose. The mean Cₘₐₓ was 269.6 ng/ml, 639.8 ng/ml, and 975.3 ng/ml at 100, 200, and 300 mg soticlestat BID respectively, which is reduced (˜ 36.9%) compared to an equivalent dose of soticlestat oral solution. The AUC₂₄ for doses 100, 200, and 300 mg BID were 562.5, 1437, and 2188 ng×h/mL, respectively.

In some embodiments, the therapeutically effective amount of soticlestat for treating neurological disease is a total daily dosage of about 200 mg, administered twice daily, and wherein the steady-state soticlestat in a patient's plasma reaches AUC₂₄ of about 563 ng×h/ml +100 ng×h/ml.

In some embodiments, the therapeutically effective amount of soticlestat for treating neurological disease is a total daily dosage of about 400 mg, administered twice daily, and wherein the steady-state soticlestat in a patient's plasma reaches AUC₂₄ of about 1437 ng×h/ml +100 ng×h/ml.

Example 10 below further describes the study's PK protocol and trial results in developmental and/or epileptic encephalopathy patients, including safety (example 9).

In one embodiment of the present invention, soticlestat in tablet formulation is administered to a patient in need of treatment for a neurological disease at a daily dosage between 100 and 600 mg. In certain embodiments, soticlestat in tablet formulation is administered to a patient at a daily dosage between 100 and 600 mg as a prophylaxis or treatment to reduce symptoms of developmental epileptic encephalopathies, epilepsy, and the like. In certain embodiments, soticlestat in a tablet formulation is administered to a patient at a daily dose between 100 and 600 mg for use as a prophylaxis or treatment for neurodegenerative disease (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like). In certain embodiments, soticlestat tablets are administered to a patient once or twice daily. In certain embodiments, soticlestat tables are administered in the fasting condition. In another embodiment, soticlestat in tablet formulation is administered to a patient in need of neurological disease treatment by selecting a dose that reaches a target steady-state soticlestat plasma AUC₂₄ between 562.5 and 2188 ng×h/mL. In certain embodiments, soticlestat in tablet formulation is administered to a patient for prophylaxis or treatment to reduce symptoms of developmental epileptic encephalopathies, epilepsy, wherein the patient's target steady-state soticlestat plasma AUC₂₄ reaches between about 460 and about 2290 ng×h/mL, e.g., between about 562.5 and about 2188 ng×h/mL. In certain embodiments, soticlestat tablet formulation is administered to a patient as a prophylaxis or treatment for neurodegenerative disease, (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like), at a dose that reaches a target steady-state soticlestat plasma AUC₂₄ between about 460 and about 2290 ng×h/mL, e.g., between about 562.5 and about 2188 ng×h/mL.

In certain embodiments, soticlestat is administered in tablet formulation to a patient in need of treatment for a neurological disease, wherein, following administration, the patient's plasma Cₘₐₓ reaches between about 220 and about 1025 ng/ml, e.g., between about 269 and about 1000 ng/mL. In certain embodiments, soticlestat is administered in tablet formulation as a prophylaxis or treatment to reduce symptoms of developmental epileptic encephalopathies, epilepsy, and the like, wherein the patient's plasma Cₘₐₓ reaches between about 220 and about 1025 ng/mL, e.g., between about 269 and about 1000 ng/mL. In certain embodiments, soticlestat is administered in tablet formulation to a patient as a prophylaxis or treatment for neurodegenerative disease (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like), wherein the patient's plasma reaches a Cₘₐₓ between about 220 and about 1025 ng/mL, e.g., between about 269 and about 1000 ng/mL. Example 10 further describes the study's PK protocol and trial results in developmental and/or epileptic encephalopathy patients, including safety data (example 9).

The blood sample schedule for collecting for PD data were similar to those collected for PK data in part A. During Part A, the mean plasma 24HC levels were decreased from baseline by 69.76% on day 11 and 76.88% on day 21 in soticlestat treated patients compared to 4.30% and 0.71% for placebo. At the end of the Part B (day 85), the mean percent decrease of plasma 24HC was 80.97%. The ability of soticlestat to lower plasma 24HC levels appears to plateau when soticlestat AUC₂₄ is greater than 800 ng×h/mL. In certain embodiments, the soticlestat dosage in tablet formulation is administered to a patient for treating neurological disease by selecting a dosage to reach a target steady-state soticlestat plasma AUC₂₄ of at least 800 ng×h/mL. In certain embodiments, the soticlestat dosage in tablet formulation is administered to a patient in need of treatment for a neurological disease by selecting a dosage that reaches a target steady-state soticlestat plasma AUC₂₄ of at least 800 ng×h/mL. In certain embodiments, the soticlestat dosage in tablet formulation is administered to a patient at a dosage to reach a target steady-state soticlestat plasma AUC₂₄ of at least 800 ng×h/mL for prophylaxis or treatment to reduce symptoms of developmental epileptic encephalopathies, epilepsy, and the like. In certain embodiments, the soticlestat dosage in tablet formulation is administered to a patient at a dosage to reach a target steady-state soticlestat plasma AUC₂₄ of at least 800 ng×h/mL for the prophylaxis or treatment of a neurodegenerative disease (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like). In some embodiments, the stead-state soticlestat plasma AUC₂₄ of the patient is from about 800 to about 1600 ng×h/mL. In some embodiments, the steady-state soticlestat plasma AUC₂₄ of the patient is from about 800 to about 1200 ng×h/mL.

In one embodiment of the invention, soticlestat tablets are administered to patient at a dose necessary to reach a targeted percent decrease in 24HC from baseline for treating neurological disease, wherein the effective 24HC percent decrease from baseline is at least 60%, at least 70%, or at least 80%. In another embodiment of the invention, soticlestat tablets are administered to patient at a dose necessary to reach a targeted percent decrease in 24HC from baseline for the prophylaxis or treatment to reduce symptoms of developmental epileptic encephalopathies, epilepsy, and the like, wherein the effective 24HC percent decrease from baseline is at least 40%, at least 50%, at least 60%, at least 70%, or at least 80%. In yet another embodiment of the invention, soticlestat tablets are administered to patient at a dose necessary to reach a targeted percent decrease in 24HC from baseline for the prophylaxis or treatment of a neurodegenerative disease (e.g., epilepsy, rare epilepsy, developmental epileptic encephalopathy, Dravet syndrome, Lennox-Gastaut syndrome, Dup15q syndrome, CDKL5 deficiency disorder, migraine, or complex regional pain syndrome and the like), wherein the effective 24HC percent decrease from baseline is at least 60%, at least 70%, or at least 80%.

Patients treated with soticlestat in Part A had a median 16.71% increase in seizure frequency from baseline, whereas placebo treated patients increased seizure frequency by 22.16% from baseline. Three patients treated concomitantly with the anti-epileptic drug perampanel had significantly increased seizure frequency. When these patients were excluded from the analysis, the soticlestat treated patients had a median 7.54% increase in seizure frequency from baseline. In part B, the median seizure frequency during days 42-85 was −36.38% from baseline and after excluding the patients taking perampanel, the median seizure frequency was −44.66%. A post-hoc analysis of the last 28 days of treatment found median seizure frequency decreased to −60.74% from baseline. Two patients had a 100% reduction in seizure frequency in the last 4 weeks of treatment. In one embodiment, a method of reducing seizure frequency in a patient comprises orally administering to the patient a therapeutically effective amount of soticlestat or a pharmaceutically acceptable salt thereof in an oral solution or a solid oral dosage form, wherein the patient achieves a reduction in seizure frequency of at least about 30% following administration.

The ELEKTRA trial was a phase 2, multicenter, randomized, double-blind, placebo-controlled study to that evaluated the safety, tolerability, efficacy of soticlestat in pediatric patients with DEE. Patients met the inclusion criteria if they were diagnosed with Dravet syndrome and had an average of ≥3 convulsive seizures per month, or if they were diagnosed with Lennox-Gastaut syndrome with an average of ≥3 convulsive or ≥4 drop seizures per a month. Other inclusion criteria were body weight ≥10 kg, currently taking a fixed dosage of 1 to 4 antiseizure medications, and failure to remain seizure free after trying at least two antiseizure medications. Convulsive seizures include focal to bilateral tonic-clonic with impaired awareness, hemi-clonic, simultaneous bilateral clonic, and generalized tonic-clonic seizures. Drop seizures can involve the entire body, trunk, or head which leads to falling. Patients were excluded from the study if they were pregnant, had a history of cataracts, or were taking the antiseizure drug parampanel. Approximately 126 patients ages ≥2 and ≤17 years were enrolled in the study. The study included two main phases, a 4-6 week screening and baseline period, and a 20-week treatment period. Patients received doses twice a day in a tablet formulation. The starting dose was adjusted according to the patient's body weight (Table 17). Patients had a two-week taper period after the treatment period and after that, a two-week safety follow-up period.

During the first 8 weeks of treatment patient dosing was a titrated for individual optimization. The remaining 12-weeks of treatment consisted of a maintenance phase. The study began with treating patients ≥9 years of age for one month before treating younger patients. Blood was collected for PK analysis on the first dosage day within one hour of the morning dose, and 30 minutes, and 1, 2, 3, and 4 hours post-dose. Subsequent blood sampling took place before the morning dose and 30 minutes post-dose. Blood samples were obtained at scheduled times for determining PK profile and Tₘₐₓ, which is the time Cₘₐₓ was reached. A patient's seizure frequency was recorded before and during the trial to calculate percent change from baseline. Soticlestat treated patients had significantly decreased seizure frequency from baseline than placebo treated patients. Example 14 describes the percent change in seizure frequency in ELEKTRA patients over time (Table 35).

The datasets from the clinical trials described above were used in a population PK and PK/PD modeling analysis. Three PK samples were excluded from the analyses because their baseline values were over the limit of quantification. The ELEKTRA dataset contained 69 single-dose and 270 steady-state dose events. Individual variables from those studies were included in the dataset to determine significant covariates and their effect size. Subject variables included the study ID, patient ID, treatment, health status, and DEE disease type. Baseline demographic variables included age, body weight, body mass index, gender, race, and Chinese or Asian ethnic origin. Baseline laboratory variables included aspartate aminotransferase (AST), gamma-glutamyl transferase (GGT), alkaline phosphatase (ALP), bilirubin, creatinine, albumin, alpha-1-acid glycoprotein (A1-AGLP), estimated glomerular filtration rate (eGFR), and creatinine clearance (CrCl)) (see formulas in example 12). Treatment variables included soticlestat formulation, crushed or crushed tablets, dosage amount, and dosing frequency. Another variable was the type of antiepileptic drugs patients were on concomitantly during the trial.

The dataset included 2193 observations from 173 subjects. Both PK and PK/PD parameters were analyzed as a mixed-effects population model using the computer program NONMEM. A mixed-effects model takes into account fixed value parameters and random-effect parameters. The first stage of the analysis began with using only data obtained from the oral solution studies in a structural and statistical base model to account for concentration changes over time and for between and within subject variability. Several different structural model types were tested and the structure of the omega complex was optimized. Omega, expressed as random effects variance, represents the difference between an individual's parameter value and the population value and describes the distribution of between subject variability across the population. The linear two-compartment model incorporates delayed first-order absorption from oral administration. Model parameters were absorption lag time (ALAG1), bioavailability (F1), elimination clearance (CL), absorption rate constant (KA), central volume (V2), inter-compartmental clearance (Q), and peripheral volume (V3). Random effects were estimated for F1, KA, Q, and V3. The omega matrix was restricted to a diagonal structure, wherein between parameter correlations are ignored. A combined additive and proportional residue error model was utilized to broadly reflect between subject variability.

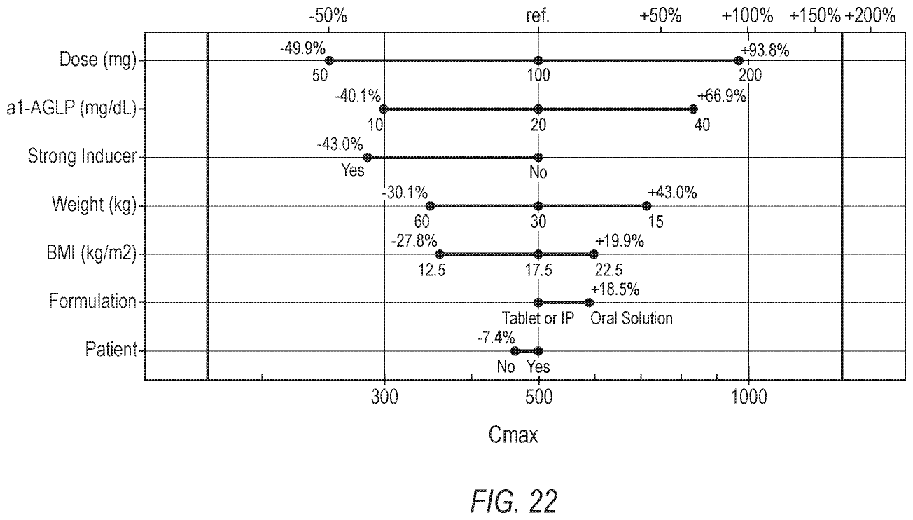

The second stage of the analysis added data collected from the tablet formulation studies in adults. Tablet formulation added covariate effects in the absorption lag time (ALAG1˜FORM) and absorption rate constant (KA˜FORM). In addition, patient differences in systemic clearance (CL) introduced variability (CL˜PATIENTS). The combined data showed a pronounced non-linearity. The non-linearity indicates that soticlestat dose and plasma concentrations are not proportional to increased dosage (see FIG. 22). This phenomenon could cause problems when adjusting dosage in patients. Covariates had significant effects on relative bioavailability (F1˜DOSE), absorption rate constant (KA˜DOSE), inter-compartmental clearance (Q-DOSE), and peripheral volume (V3˜DOSE).

The third stage of the analysis added the pediatric data collected from the ELEKTRA study. A range of different formulations and administration procedures were used for the pediatric patients, including swallowed mini-tablets, tablets, and crushed tablets administered through a gastrostomy tube. However, the tablet formulation model fit the ELEKTRA dataset best. Next variables were screened separately by correlating the individual random effects with the base model. An F-test was used to determine the significance of the individual random effects with and without the variable. Candidates were further screened using a standard forward inclusion (α<0.05), backward deletion (α<0.01) procedure. Growth-related changes, such as age, body weight, and body mass index affected PK parameters. The data was best described with the covariate effects of body weight on relative bioavailability (F1˜WEIGHT) and body mass index on absorption rate constant in subjects 18 years or younger (KA˜BMI (AGE≤18). The final population PK model included the following covariates: baseline A1-AGLP on relative bioavailability, body weight on relative bioavailability, dose on KA, F1, Q, and V3, patient status on CL, BMI on KA (age≤18), strong PK inducing antiepileptic comedications on KA, eGFR on V3, and Asian origin on V3. In some embodiments, a patient's dosage is adjusted based on an individual's covariate values, said covariates comprising body weight, body mass index, antiepileptic comedication(s), A1-AGLP, eGFR level, formulation type, regimen, and race. In one embodiment, a patient's baseline plasma A1-AGLP level guides the adjustment of the soticlestat dosage. In one embodiment, a patient's concomitant usage of antiepileptic drugs guides the adjustment of the soticlestat dosage. In one embodiment, a patient's measure of kidney function, comprising eGFR levels, guides the adjustment of soticlestat dosage. In yet another embodiment, dosage is adjusted based on whether a patient's ethnic origin is Asian or Chinese. In one embodiment, soticlestat dosage is adjusted based on the patient's body mass index or body weight for patients≤18 years of age.

The final population PK parameters are shown in Table 25. The PK model parameters can be used as a tool to estimate PK and drug exposure in an individual or a population (such as with ELEKTRA), as well as to calculate individualized dosing based on covariates. For example, optimal daily dosing can be calculated using the area under the concentration time curve and clearance values (dose=targetAUC₂₄×CL). Blood sampling at steady-state was limited in the ELEKTRA study, thus some of the PK parameters shown in Table 32 for the ELEKTRA population were simulated by interpolation.

Similar to the PK model, the PK/PD model was developed as a population mixed-effects model and incorporated the individual PK parameters from the final PK population model. A turnover model was used to characterize changes in plasma 24HC overtime. Plasma 24HC was considered to be at steady-state baseline (BL) before the start of treatment. The PK/PD model parameters include a plasma/brain scaling parameter (KPLBR), maximum inhibition of 24CH synthesis (Iₘₐₓ), concentration resulting in 50% enzyme inhibition (IC50), and shape parameter. PK/PD covariates were screened in a procedure similar to the PK covariates. In the final PK/PD model, two covariates, A1-AGLP and body weight affected baseline 24HC. Example 13 describes the PK/PD modeling procedure and the final PK/PD parameter estimates are shown in Table 31. The derived PK/PD parameters for the ELEKTRA dataset are shown in Table 33.

In one embodiment of the present invention, soticlestat is administered orally at a daily dosage between about 80 and about 600 mg to pediatric patients in need of treatment for a neurological disease. In certain embodiments, soticlestat is administered orally at a dosage between about 80 and about 600 mg to pediatric patients for prophylaxis or treatment to reduce symptoms of developmental epileptic encephalopathies, epilepsy, and the like. In certain embodiments, soticlestat is administered orally at a dosage between about 80 and about 600 mg to pediatric patients to treat traumatic brain injury or stroke.

Pediatric doses of soticlestat can be selected according to the following table.

| | |
|---|---|
| Pediatric Body | Adult Reference Dose |

| Weight | 100 mg BID | 200 mg BID | 300 mg BID |
|---|---|---|---|
| 10 to < 15 kg | 40 mg BID | 60 mg BID | 100 mg BID |
| 15 to < 30 kg | 60 mg BID | 120 mg BID | 200 mg BID |
| 30 to < 45 kg | 80 mg BID | 140 mg BID | 200 mg BID |
| ≥45 kg | 100 mg BID | 200 mg BID | 300 mg BID |

In some embodiments, a dose of 40 mg BID is administered to a pediatric patient, e.g., weighing about 10-15 kg. In some embodiments, a dose of 60 mg BID is administered to a pediatric patient, e.g., weighing about 10-15 kg. In some embodiments, a dose of 100 mg BID is administered to a pediatric patient, e.g., weighing about 10-15 kg. In some embodiments, a dose of 60 mg BID is administered to a pediatric patient, e.g., weighing about 15-30 kg. In some embodiments, a dose of 120 mg BID is administered to a pediatric patient, e.g., weighing about 15-30 kg. In some embodiments, a dose of 200 mg BID is administered to a pediatric patient, e.g., weighing about 15-30 kg. In some embodiments, a dose of 80 mg BID is administered to a pediatric patient, e.g., weighing about 30-45 kg. In some embodiments, a dose of 1400 mg BID is administered to a pediatric patient, e.g., weighing about 30-45 kg. In some embodiments, a dose of 200 mg BID is administered to a pediatric patient, e.g., weighing about 30-45 kg. In some embodiments, a dose of 100 mg BID is administered to a pediatric patient, e.g., weighing 45 kg or more. In some embodiments, a dose of 200 mg BID is administered to a pediatric patient, e.g., weighing 45 kg or more. In some embodiments, a dose of 300 mg BID is administered to a pediatric patient, e.g., weighing 45 kg or more. In some embodiments, the pediatric patient weighing 45 kg or more is an adolescent patient. In some embodiments, the pediatric patient weighing about 30-45 kg is an adolescent patient.

In certain embodiments, soticlestat is administered to non-Chinese pediatric patients in need of treatment for neurological disease at a dose selected to reach a targeted soticlestat plasma AUC₂₄ between 1430 and 4770 ng×h/mL. In certain embodiments, soticlestat is administered to Chinese or Asian pediatric patients in need of treatment for neurological disease at a dose selected to reach a targeted soticlestat plasma AUC₂₄ between 1390 and 4030 ng×h/mL.

In certain embodiments, soticlestat is administered to non-Chinese pediatric patients for prophylaxis or treatment to reduce symptoms of developmental epileptic encephalopathies, epilepsy, and the like, at a dose selected to reach a targeted soticlestat plasma AUC₂₄ between 1430 and 4770 ng×h/mL. In certain embodiments, soticlestat is administered to Chinese or Asian pediatric patients for prophylaxis or treatment to reduce symptoms of developmental epileptic encephalopathies, epilepsy, and the like at a dose selected to reach a targeted soticlestat plasma AUC₂₄ between 1390 and 4030 ng×h/mL.

In certain embodiments, soticlestat is administered to non-Chinese pediatric patients for traumatic brain injury or stroke at a dose selected to reach a targeted soticlestat plasma AUC₂₄ between 1430 and 4770 ng×h/mL. In certain embodiments, soticlestat is administered to Chinese or Asian pediatric patients for traumatic brain injury or stroke at a dose selected to reach a targeted soticlestat plasma AUC₂₄ between 1390 and 4030 ng×h/mL.

In some embodiments, soticlestat is administered to pediatric patients at a dosage selected to reach a targeted percent decreased in 24HC plasma from baseline between 68% to 83%. In certain embodiments, soticlestat is administered to pediatric patients twice daily. In some embodiments, soticlestat is administered in an oral tablet formulation. In other embodiments, soticlestat is administered as an oral solution formulation. In some embodiments, soticlestat is administered in the fasting state.

In certain embodiments, the disclosed soticlestat PK and PK/PD parameters are used to determine the optimal soticlestat dose to administer in a subject for treatment of disease characterized by neuronal hyperactivity or epileptiform activity, e.g., Alzheimer's disease, mild cognitive disorder, Huntington's disease, Parkinson's disease, amyotrophic lateral sclerosis, traumatic brain injury, cerebral infarction, glaucoma, multiple sclerosis, and the like. In some embodiments, soticlestat is administered in a solid form, which may include a tablet or capsule formulation.

Treatments for neurological and neurodegenerative diseases are described herein. In some embodiments, the neurological disease is epilepsy, rare epilepsy, developmental epileptic encephalopathy, epileptic encephalopathy, Dravet syndrome, Lennox-Gastaut syndrome, focal onset seizures, major motor seizures, major motor drop seizures, focal seizures with secondary generalization, infantile spasms, primary generalized tonic-clonic seizures, partial onset seizures with our without secondary generalization, simple partial seizures, complex partial seizures, simple absence seizures, complex absence seizures, Dup15q syndrome, CDKL5 deficiency disorder, migraine, or complex regional pain syndrome. In some embodiments, the disease is developmental epileptic encephalopathy or epileptic type disease. In some embodiments, the disease is Dravet syndrome or Lennox-Gastaut syndrome. In some embodiments, the disease is Alzheimer's disease, mild cognitive disorder, Huntington's disease, Parkinson's disease, amyotrophic lateral sclerosis, traumatic brain injury, cerebral infarction, glaucoma, or multiple sclerosis.

The IUPAC name for soticlestat shown below is 4-benzyl-4-hydroxypiperidin-1-yl) (2,4′-bipyridin-3-yl) methanone.

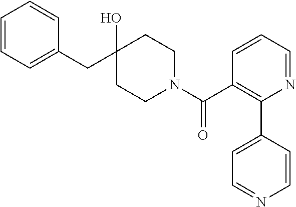 [SMILES: O=C(c1cccnc1-c1ccncc1)N1CCC(O)(Cc2ccccc2)CC1]

Soticlestat may be prepared according to methods known in the art, including those described in U.S. publication numbers US 2014/0228373 A1, published Aug. 14, 2014 and US 2018/0297980 A1, published Oct. 18, 2018.

Crystalline polymorphs of soticlestat include Form I, II, and 3.0 hydrate. The production methods for Forms I, II, and 3.0 hydrate, and their powder X-ray diffraction profiles, are described in example 1.

In some embodiments, soticlestat is formulated as a solid using ingredients typically used for tablet formulation.

In some embodiments, soticlestat is prepared as a pharmaceutically acceptable salt. Examples of a pharmaceutically acceptable salt forming counterions include inorganic acid, organic acid, and acidic amino acid, and the like. Preferable examples of the salt with inorganic acid include salts with hydrochloric acid, hydrobromic acid, nitric acid, sulfuric acid, phosphoric acid and the like. Preferable examples of the salt with organic acid include salts with formic acid, acetic acid, trifluoroacetic acid, phthalic acid, fumaric acid, oxalic acid, tartaric acid, maleic acid, citric acid, succinic acid, malic acid, benzoic acid, methanesulfonic acid, benzenesulfonic acid, p-toluene-sulfonic acid and the like. Preferable examples of the salt with acidic amino acid include salts with aspartic acid, glutamic acid and the like. Methods for producing a pharmaceutically acceptable salt from inorganic acid, organic acid, or acidic amino acid is well known by persons having ordinary skill in the art.

### Definitions

The term “soticlestat” includes various forms of soticlestat such as pharmaceutically acceptable salt(s), hydrate(s), solvate(s), and polymorph(s), thereof. Soticlestat is also known as TAK-935.

The term “covariates” are the variables that affect PK and PD parameters and include continuous or categorical variables, such as age, weight, body mass index (BMI), concomitant drug use, race, health status, and liver and kidney function.

The term “F1” refers to “relative bioavailability”, which is the soticlestat concentration that enters systemic circulation.

The term “KA” is the absorption rate constant and refers to the rate at which soticlestat enters the circulation.

The term “ALAG1” is the absorption lag time.

The term “BSV” refers to the between subject variability.

The term “V2” refers to central volume.

The term “V3” refers to peripheral volume.

The term “Q” refers to the inter-compartmental clearance.

The term “RV” refers to the residual variability and represents the composite variability derived from inter-assay variability, intra-individual variability, errors in timing and dosing, subject non-compliance, and other unexplained errors.

The PK parameters can be measured after administration of a single dose of the composition or after administration of multiple doses of the composition such that a steady state concentration of soticlestat is attained.

The term “steady state” concentration relates to the concentration obtained after administration of soticlestat on a daily basis for at least 14 days.

The term “escalating doses” or “ascending dose” relates to increasing the concentration of the subsequent dosage to a subject.

The term “M-I” refers to the N-oxide metabolite of soticlestat.

The term “Cₘₐₓ” is defined as the concentration of soticlestat in the plasma at the point of maximum concentration.

The term “Tₘₐₓ” refers to the time at which soticlestat in the plasma is at the highest concentration.

The term “AUC” represents “area under the concentration-time curve” with respect to a plot of drug concentration in plasma versus time from ingestion. AUC provides a measure of the total drug exposure and is expressed as ng×h/mL. An AUC interval is from time zero to any time ‘t’ post drug administration or if extrapolated to infinity the time of the last quantifiable concentration. For instance, the term “AUC_{(X-Y)}” is the area under the curve in a plot of concentration of drug in plasma over time, measured from time point X hours after administration of the composition to time point Y hours after administration of the composition. AUC₂₄ is the exposure of plasma drug concentration over 24 hours.

The term “CV %” is the coefficient of variation is a measure of the data points around the mean and is calculated from the formula: ((standard deviation/mean)×100).

The term “t1/2z” is the terminal elimination half-life of soticlestat.

The term “V2/F” stands for the apparent volume of distribution during the terminal phase after extravascular administration.

The term “CL” is a constant relating the rate of elimination (mg/h) to the volume of units of plasma concentration (Cp) in mg/L. The units for CL are L/h. The term “CLR” is the renal clearance of the drug. The term “CL/F” stands for the time of total clearance of soticlestat from plasma after oral administration expressed as volume/time/kg.

The term “fₑ” is the fraction of dose excreted unchanged into urine.

The term metabolic ration (MR) is the calculated from the formula: [AUC_{∞} (M-I)×soticlestat molecular weight]/[AUC_{∞}(soticlestat)×M-I molecular weight]-1).

The term ‘BID’ refers to a patient receiving a dose twice a day, i.e., twice daily. The term “QD” refers to a patient receiving a dose once a day, i.e., once daily.

The term “Aer” is a measure of the cumulative amount of soticlestat excreted in urine over time. For QD dosing, the term Ae24 stands for the cumulative amount of unchanged soticlestat excreted in urine over 24 hours. For BID dosing, Ae12 stands for the cumulative amount of unchanged soticlestat excreted over 12 hours.

The term “Et” is the observed effect at time t; “E12” is the observed effect at 12 hours; “E24” is the observed effect at 24 hours.

The term “AUEC_{τ}” for a given time (t) measures the change in 24HC concentration over time. The term “AUEC₁₂” stands for the area under the effect-time curve from 0 to 12 hours. The term “AUEC₂₄” is the area under the effect-time curve from 0 to 24 hours.

The term “confidence interval” refers to the percent likelihood that the population mean lies within an upper and lower interval.

The term “strong inducer” for a concomitant anti-epileptic drug means that it increases soticlestat-mediated CH24H enzyme inhibition, which appears related to an increase in the absorption rate.

The term “solid” used herein defines the matter state of soticlestat. Solid soticlestat may be in the form of a tablet or capsule.

### Examples

### Example 1. Soticlestat polymorph production methods and characterization

Crystalline polymorphs of soticlestat (4-benzyl-4-hydroxypiperidin-1-yl) (2,4′-bipyridin-3-yl) methanone) include Form I, Form II, and 3.0 hydrate. The following are methods for producing each of these soticlestat polymorphs.

### Form I

Form I was produced by stirring a mixture of ethyl 2-chloronicotinate (16.2 g), pyridine-4-boronic acid (12.9 g), sodium carbonate (27.8 g), tetrakis (triphenylphosphine) palladium (0) (5.04 g), water (50.0 mL) and 1,2-dimethoxyethane (250 mL) overnight at 100° C. under a nitrogen atmosphere. The reaction mixture was diluted with water and extracted with ethyl acetate. The extract was washed with saturated brine, and dried over anhydrous sodium sulfate. The solvent was evaporated under reduced pressure and purified by silica gel column chromatography (NH, ethyl acetate/hexane) to yield 14.1 g of ethyl 2,4′-bipyridine-3-carboxylate. The proton nuclear magnetic resonance spectrum was measured by Fourier-transform ¹H NMR (300 MHz, CDCl₃) δ 1.09 (3H, t, J=7.0 Hz), 4.19 (2H, q, J=7.0 Hz), 7.39-7.48 (3H, m), 8.21 (1H, dd, J=7.8, 1.7 Hz), 8.66-8.74 (2H, m), 8.81 (1H, dd, J=4.7, 1.7 Hz).

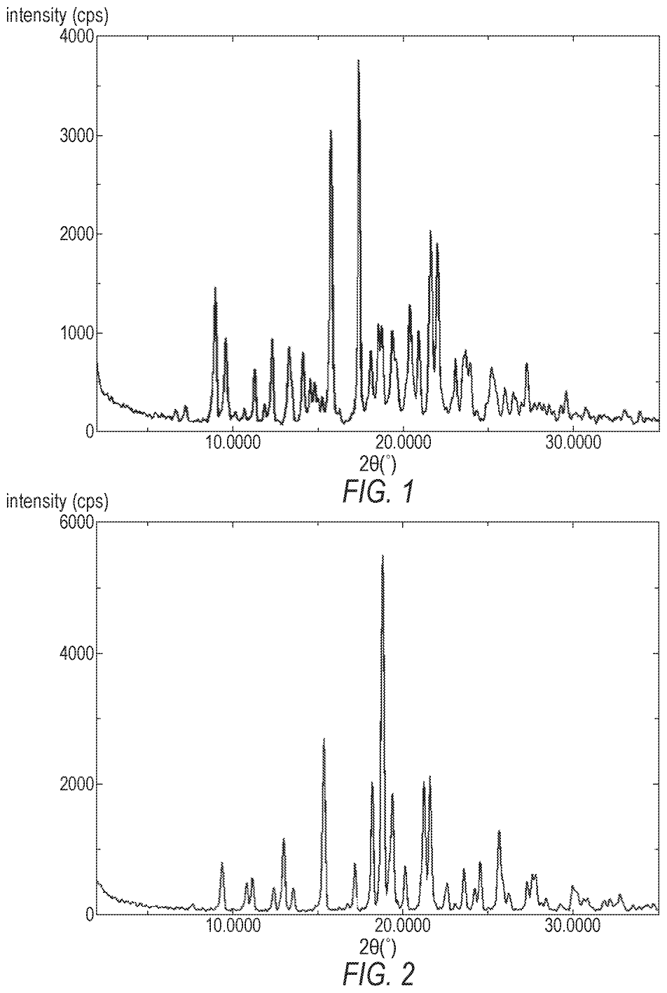

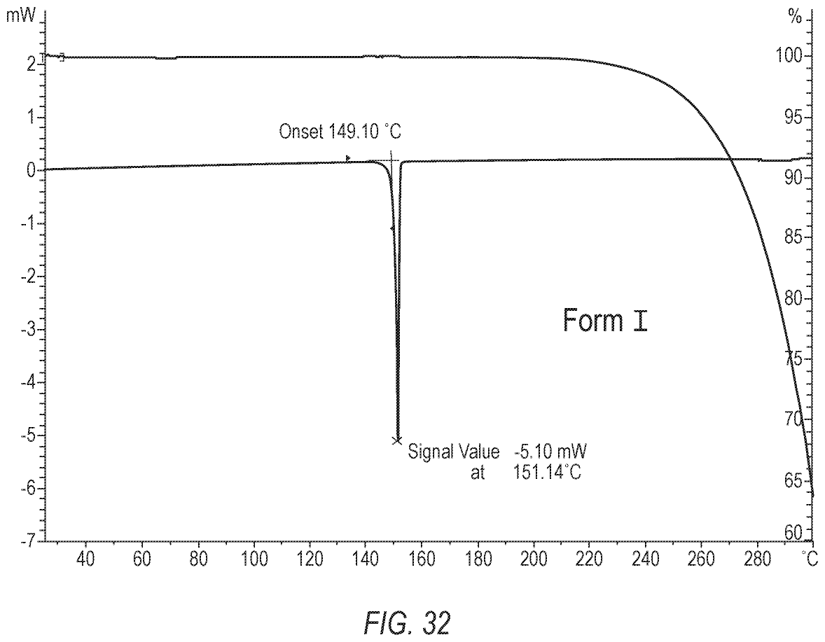

The ethyl 2,4′-bipyridine-3-carboxylate (14.1 g) was mixed with 6 N hydrochloric acid (200 mL) and heated under reflux overnight. Toluene was then added to the mixture and evaporated under reduced pressure to yield 16.4 g of 2,4′-bipyridine-3-carboxylic acid dihydrochloride (MS (atmospheric chemical ionization+): molecular ion peak-[M+H]⁺201.1). Next, a suspension of 2,4′-bipyridine-3-carboxylic acid dihydrochloride (5.0 g), 4-benzyl-4-hydroxypiperidine (3.9 g), 2-(1H-7-azabenzotriazol-1-yl)-1,1,3,3-tetramethyl uranium hexafluorophosphate (HATU) (10 g) and triethylamine (13 mL) in N,N-dimethylformamide (50 mL) was stirred overnight at room temperature. The reaction mixture was then diluted with water and extracted with ethyl acetate. The extract was washed with saturated brine and dried over anhydrous sodium sulfate under reduced pressure. The resulting residue was purified by silica gel column chromatography (NH, ethyl acetate/hexane), and recrystallized from ethyl acetate/hexane to give the 3.2 g of (4-benzyl-4-hydroxypiperidin-1-yl) (2,4′-bipyridin-3-yl) methanone. The proton nuclear magnetic resonance spectrum was measured by Fourier-transform ¹H NMR (300 MHz, CDCl₃) δ 0.06-1.74 (5H, m), 2.34-3.18 (5H, m), 4.42-4.60 (1H, m), 6.98-7.15 (2H, m), 7.21-7.34 (3H, m), 7.41 (1H, dd, J=7.6, 4.9 Hz), 7.61 (1H, d, J=5.3 Hz), 7.70-7.83 (2H, m), 8.62-8.81 (3H, m). The melting point of Form I was 150-152° C. FIG. 1 shows the X-ray powder diffraction pattern of pure crystalline Form I. Table 1 lists the 20 peak positions and the interplanar d spacing (d-value) for the X-ray diffraction pattern of Form I. FIG. 32 shows the DSC trace/TGA thermogram for Form I.

The powder X-ray diffractions in Tables 1-3 and FIGS. 1-3 were measured under the following conditions:
Measurement apparatus: RIGAKU Ultima IV
Measurement Conditions:
tube voltage: 40 kV
tube current: 50 mA
scan speed: 6°/min
scan angle (2θ): 2-35°

TABLE 1
Crystal X-ray diffraction 2θ peak
position and d value of Form I soticlestat

| 2θ (°) | d value(Å) |
|---|---|
| 8.98 | 9.84 |
| 9.58 | 9.22 |
| 11.28 | 7.84 |
| 12.30 | 7.19 |
| 14.10 | 6.28 |
| 15.74 | 5.63 |
| 17.38 | 5.10 |
| 20.90 | 4.25 |
| 21.58 | 4.11 |
| 22.00 | 4.04 |

In some embodiments, Form I is characterized by an x-ray powder diffraction pattern comprising five or more, six or more, seven or more, eight or more, nine or more, or ten peak 2θ values selected from 9.0, 9.6, 11.3, 12.3, 14.1, 15.7, 17.4, 20.9, 21.6, and 22.0±0.2°2θ.

In some embodiments, Form I is characterized by an x-ray powder diffraction pattern comprising peak 2θ values at 9.0, 9.6, 11.3, 12.3, 14.1, 15.7, 17.4, 20.9, 21.6, and 22.0±0.2°2θ.

In some embodiments, Form I is characterized by an x-ray powder diffraction pattern comprising at least three peak 2θ values selected from 9.0, 9.6, 14.1, 15.7 and 17.4±0.2°2θ.

In some embodiments, Form I is characterized by an x-ray powder diffraction pattern comprising peak 2θ values at 9.0, 9.6, 14.1, 15.7 and 17.4±0.2°2θ.

### Form II

The soticlestat Form II polymorph is more stable than Form I and was the polymorph used in the PK and PD studies described in the examples below. Form II was produced by adding thionyl chloride (47.9) to a mixture of 2-chloronicotinic acid (53.7 kg), toluene (252 kg) and N,N-dimethylformamide (0.55 kg) and stirring under atmospheric nitrogen at 90° C. for 1 hr. The reactant was concentrated under reduced pressure, and the resulting residue dissolved in tetrahydrofuran (216 kg) and 2 M aqueous sodium hydroxide solution (419 kg). To this solution, 4-benzyl-4-hydroxypiperidine benzoate (97.0 kg) was added and the mixture stirred under atmospheric nitrogen at room temperature for 1 hour, after which 10% aqueous potassium carbonate solution (291 kg) was added. The mixture was then extracted with ethyl acetate (524 kg). Ethanol (46 kg) and n-heptane (365 kg) were added and the solution and concentrated under reduced pressure resulted in (4-benzyl-4-hydroxypiperidin-1-yl) (2-chloropyridin-3-yl) methanone (96.8 kg) as crystals (MS (electron spray ionization+): molecular ion peak-[M+H]⁺331.1).

The (4-benzyl-4-hydroxypiperidin-1-yl) (2-chloropyridin-3-yl) methanone (60.0 kg) was added to bis (acetylacetonato) palladium (II) (0.28 kg), tris (4-methoxyphenyl)phosphine (0.32 kg), pyridine-4-boronic acid (27.9 kg), sodium carbonate (57.6 kg), tertbutanol (482 kg) and water (300 kg) and stirred overnight under nitrogen atmosphere at 85° C. The following day, 20% aqueous sodium hydrogensulfite solution (600 kg) was added to the reaction mixture and extracted with ethyl acetate (271 kg). The extract was washed with 1M hydrochloric acid (611 kg), 20% aqueous potassium carbonate solution (480 kg) and 10% aqueous potassium carbonate solution (300 kg), and the solvent evaporated under reduced pressure. Acetone (143 kg) and n-heptane (206 kg) were then added to produce the residue (4-benzyl-4-hydroxypiperidin-1-yl) (2,4′-bipyridin-3-yl) methanone crystals (58.1 kg). The proton nuclear magnetic resonance spectrum was measured by Fourier-transform ¹H NMR (300 MHZ, CDCl3) 8 0.05-1.73 (5H, m), 2.34-2.53 (1H, m), 2.61-3.25 (4H, m), 4.37-4.64 (1H, m), 6.96-7.16 (2H, m), 7.19-7.34 (3H, m), 7.42 (1H, dd, J=7.6, 4.9 Hz), 7.54-7.85 (3H, m), 8.60-8.83 (3H, m).

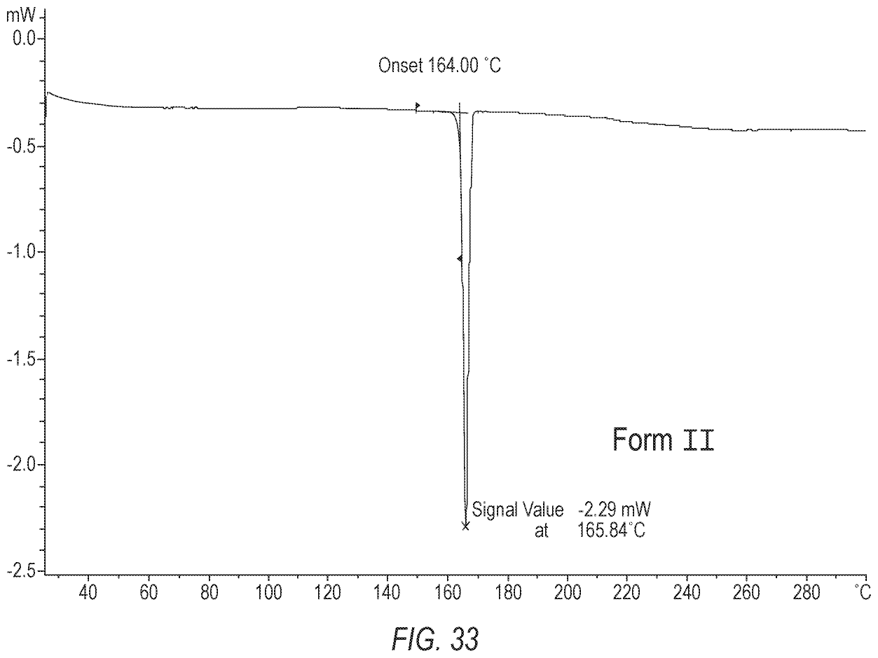

The seed crystals were produced by adding (4-benzyl-4-hydroxypiperidin-1-yl) (2,4′-bipyridin-3-yl) methanone (66.4 kg) to n-heptane (109 kg), ethyl acetate (216 kg), and ethanol (52.4 kg), and dissolving the compound under nitrogen atmosphere at 70° C. The dissolved compound was cooled to room temperature and n-heptane (58.1 kg) added to give (4-benzyl-4-hydroxypi-peridin-1-yl) (2,4′-bipyridin-3-yl) methanone crystals. The crystals were pulverized using a jet mill to yield seed crystals. Next, (4-benzyl-4-hydroxypiperidin-1-yl) (2,4′-bipyridin-3-yl) methanone (60.0 g) was dissolved in ethanol (118.4 g) at 70° C. To this solution was added n-heptane (41.0 g) and the seed crystals (120 mg). The further addition of n-heptane (451.4 g) yielded the Form II crystalline polymorph (57.2 g). The Form II polymorph melting point was 164° C. when measured under atmospheric nitrogen gas at a temperature rise rate of 5° C./min using a METTLER TOLEDO apparatus. Table 2 shows the 20 and d-spacing value of the powder X-ray diffraction peaks of Form II crystalline soticlestat. The X-ray powder diffraction pattern of Form II is shown in FIG. 2. FIG. 33 shows the DSC trace for Form II.

TABLE 2
Crystal X-ray diffraction 2θ peak
position and d value of Form II soticlestat

| 2θ (°) | d value(Å) |
|---|---|
| 9.38 | 9.42 |
| 10.82 | 8.17 |
| 12.98 | 6.81 |
| 15.34 | 5.77 |
| 17.16 | 5.16 |
| 18.20 | 4.87 |
| 18.80 | 4.72 |
| 19.38 | 4.58 |
| 20.14 | 4.41 |
| 21.58 | 4.11 |

In some embodiments, Form II is characterized by an x-ray powder diffraction pattern comprising five or more, six or more, seven or more, eight or more, nine or more, or ten peak 2θ values selected from 9.4, 10.8, 13.0, 15.3, 17.2, 18.2, 18.8, 19.4, 20.1, and 21.6±0.2°2θ.

In some embodiments, Form II is characterized by an x-ray powder diffraction pattern having peak 2θ values at 9.4, 10.8, 13.0, 15.3, 17.2, 18.2, 18.8, 19.4, 20.1, and 21.6±0.2°2θ.

In some embodiments, Form II is characterized by an x-ray powder diffraction pattern having at least three peak 2θ values selected from 9.4, 13.0, 15.3, 17.2, and 18.8±0.2°2θ.

In some embodiments, Form II is characterized by an x-ray powder diffraction pattern having peak 2θ values at 9.4, 13.0, 15.3, 17.2, and 18.8±0.2°2θ.

### 3.0 Hydrate

The soticlestat 3.0 hydrate polymorph was produced by adding 2-chloronicotinic acid (10.0 kg), toluene (43.4 kg) and 1,2-dimethoxyethane (43.4 kg) to thionyl chloride (9.1 kg), and stirred under atmospheric nitrogen at 80° C. for 3 hr. The reaction mixture was concentrated under reduced pressure, and the residue dissolved in tetrahydrofuran (88.9 kg). Thereafter potassium tert-butoxide (8.4 kg) and tetrahydrofuran (88.9 kg) were added at −5° C. Next, aqueous sodium chloride solution was added to the mixture and the mixture was extracted with toluene. The extract was concentrated under reduced pressure, and 1,2-dime-thoxyethane was added, producing tert-butyl 2-chloronicotinate (13.1 kg).

The tert-butyl 2-chloronicotinate (9.3 kg) was added, along with pyridine-4-boronic acid (6.4 kg) and tetrakistriphenylphosphine palladium (1.5 kg), to a mixture of sodium carbonate (13.8 kg), 1,2-dime-thoxyethane (80.7 kg) and water (93.0 kg), and stirred under atmospheric nitrogen at 80° C. for 24 hr. Ethyl acetate was added to the mixture, and the mixture concentrated under reduced pressure. Thereafter, sodium chloride was added and the mixture was extracted with ethyl acetate. The extract was concentrated under reduced pressure to produce tert-butyl (2,4′-bipyridine)-3-carboxylate (10.6 kg).

A mixture of tert-butyl (2,4′-bipyridine)-3-carboxylate (10.6 kg) and ethyl acetate (57.4 kg) were added to 6M hydrochloric acid and 4N hydrochloric acid/ethyl acetate solution, and the mixture was stirred under nitrogen atmosphere at 25° C. for 22 hr to give (2,4′-bipyridine)-3-carboxylic acid dihydrochloride (11.1 kg).

The (2,4′-bipyridine)-3-carboxylic acid dihydrochloride (11.1 kg) was added to a mixture of methanol (79.2 kg) and water (11.0 kg), and dissolved under atmospheric nitrogen at 65° C. The solution was cooled to room temperature, and the addition of ethyl acetate resulted in (2,4′-bipyridine)-3-carboxylic acid crystals (7.9 kg).

The (2,4′-bipyridine)-3-carboxylic acid hydrochloride (7.5 kg) was added to tetrohydrofuran (51.6 kg), diisopropylethylamine (6.1 kg), 4-benzyl-4-hydroxypiperidine (6.7 kg), 1-hydroxyben-zotriazole (0.5 kg) and 1-ethyl-3-(3-dimethylaminopropyl) carbodiimide hydrochloride (7.3 kg), and the mixture was stirred under nitrogen atmosphere at 25° C. for 3 hr. Aqueous potassium carbonate solution was added to the reaction mixture, and the mixture was extracted with ethyl acetate. The extract was concentrated under reduced pressure, and n-heptane was added to yield (4-benzyl-4-hydroxypiperidin-1-yl) (2,4′-bipyridin-3-yl) methanone (10.5 kg).

The (4-benzyl-4-hydroxypiperidin-1-yl) (2,4′-bipyridin-3-yl) methanone (10.0 kg) was added to isopropyl alcohol (39.1 kg) and the compound was dissolved under atmospheric nitrogen at 70° C. The solution was concentrated under reduced pressure, and water was added to yield the crystals (8.3 kg) of (4-benzyl-4-hydroxypiperidin-1-yl) (2,4′-bipyri-din-3-yl) methanone. This is crystalline Form I and thus, this method describes a second production process for Form I.

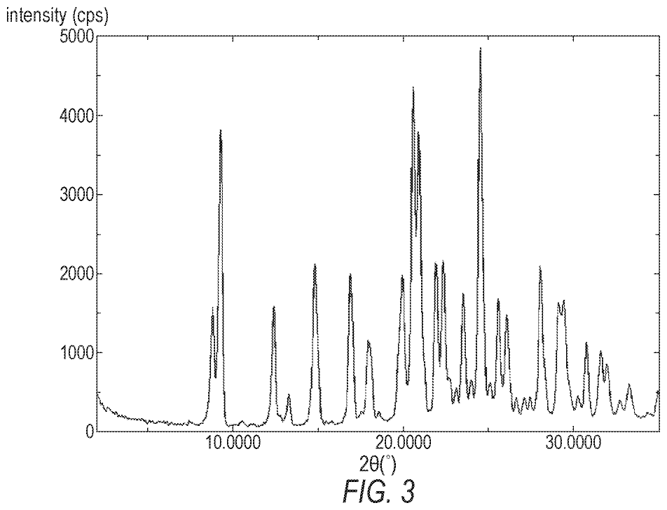

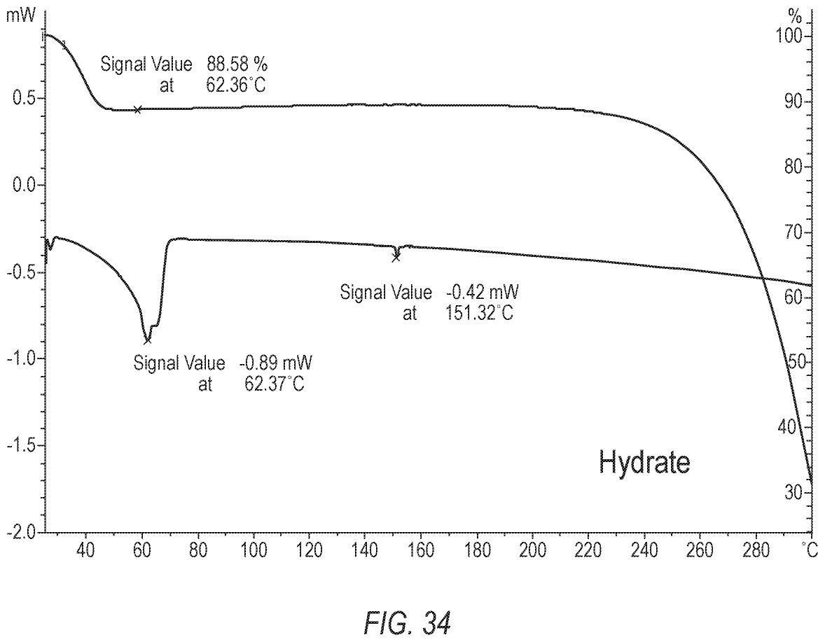

The (4-benzyl-4-hydroxypiperidin-1-yl) (2,4′-bipyri-din-3-yl) methanone crystals were pulverized using jet mill. The melting point of the milled product was measured under nitrogen gas at a temperature rise rate of 5° C./min using a METTLER TOLEDO apparatus. The resulting (4-benzyl-4-hydroxypiperidin-1-yl) (2,4′-bipyridin-3-yl) methanone melting point was 164° C. To obtain 3.0 hydrate crystals, (4-benzyl-4-hydroxypiperidin-1-yl) (2,4′-bipyridin-3-yl) methanone (about 9 g) was added to 0.5% (w/v) aqueous methyl cellulose solution (about 60 mL), and mixed using a conditioning mixer at room temperature for 5 min. The mixture was refrigerated for 2 days and the resulting crystals in the suspension were collected by filtration. A moisture measurement (Karl Fischer moisture measurement) was taken from a ˜2 mg sample at room temperature (about 26° C.) at a relative humidity of about 35% using a Hiranuma Sangyo Co., Ltd. AQ-7 electrolyte Aqualyte RS-A apparatus. The result indicated that the crystal was (4-benzyl-4-hydroxypiperidin-1-yl) (2,4′-bipyridin-3-yl) methanone trihydrate. Table 3 shows the 20 and d-spacing value of the powder X-ray diffraction peaks for soticlestat 3.0 hydrate crystalline. The X-ray powder diffraction pattern of soticlestat 3.0 hydrate is shown in FIG. 3. FIG. 34 shows the DSC trace/TGA thermogram for the 3.0 hydrate.

TABLE 3
Crystal X-ray diffraction 2θ peak position
and d value of 3.0 hydrate soticlestat

| 2θ (°) | d value(Å) |
|---|---|
| 8.84 | 9.99 |
| 9.30 | 9.50 |
| 12.42 | 7.12 |
| 14.78 | 5.99 |
| 16.88 | 5.25 |
| 20.54 | 4.32 |
| 20.90 | 4.25 |
| 21.90 | 4.06 |
| 22.32 | 3.98 |
| 24.48 | 3.63 |

In some embodiments, crystalline 3.0 hydrate of soticlestat is characterized by an x-ray powder diffraction pattern comprising five or more, six or more, seven or more, eight or more, nine or more, or ten peak 2θ values selected from 8.8, 9.3, 12.4, 14.8, 16.9, 20.5, 20.9, 21.9, 22.3, and 24.5±0.2°2θ.

In some embodiments, crystalline 3.0 hydrate of soticlestat is characterized by an x-ray powder diffraction pattern comprising peak 2θ values at 8.8, 9.3, 12.4, 14.8, 16.9, 20.5, 20.9, 21.9, 22.3, and 24.5±0.2°2θ.

In some embodiments, crystalline 3.0 hydrate of soticlestat is characterized by an x-ray powder diffraction pattern comprising at least three peak 2θ values selected from 9.3, 12.4, 14.8, 16.9, and 20.5±0.2°2θ.

In some embodiments, crystalline 3.0 hydrate of soticlestat is characterized by an x-ray powder diffraction pattern comprising peak 2θ values at 9.3, 12.4, 14.8, 16.9, and 20.5±0.2°2θ.

Tablets comprising soticlestat drug substance, such as Form I, Form II, 3.0 hydrate, or any mixture thereof, may be prepared according to methods known in the art. One exemplary tablet formulation is provided in Table 4.

TABLE 4
Ingredients and amounts for tablet formulation

| Ingredient | Amount |
|---|---|
| 1. soticlestat | 30 g |
| 2. lactose | 50 g |
| 3. cornstarch | 15 g |
| 4. calcium carboxymethocellulose | 40 g |
| 5. magnesium stearate | 1 g |
| | 140 g |

### Example 2. Bioavailability of single escalating doses of soticlestat

Healthy men and women received single ascending doses of soticlestat oral solution to access the safety and tolerability of dose concentrations. The study enrolled 48 subjects in the single rising dose study. Subjects were sorted into six cohorts and randomly assigned to treatment or placebo groups. Table 5 below shows subject demographics for the single rising dose study.

TABLE 5
Subject demographics and baseline characteristics
Single Rising Dose Study

| | | | Soticlestat | | | |
|---|---|---|---|---|---|---|
| | Placebo | | (all arms) | | Total | |
| Characteristic | (n = 12) | | (n = 36) | | (n = 48) | |
| Age, | 35.9 | (10.41) | 32.8 | (10.10) | 33.6 | (10.15) |
| mean (SD), | | | | | | |
| years | | | | | | |
| Sex, n (%) | | | | | | |
| Male | 7 | (58.3) | 19 | (52.8) | 26 | (54.2) |
| Female | 5 | (41.7) | 17 | (47.2) | 22 | (45.8) |
| Race, n (%) | | | | | | |
| White | 10 | (83.3) | 29 | (80.6) | 39 | (81.3) |
| Black or | 2 | (16.7) | 6 | (16.7) | 8 | (16.7) |
| African | | | | | | |
| American | | | | | | |
| Multiracial | 0 | | 1 | (2.8) | 1 | (2.1) |
| Ethnicity, | | | | | | |
| n (%) | | | | | | |
| Hispanic or | 1 | (8.3) | 3 | (8.3) | 4 | (8.3) |
| Latino | | | | | | |
| Non-Hispanic or - | 11 | (91.7) | 33 | (31.7) | 44 | (91.7) |
| Latino | | | | | | |
| Height, | 170.8 | (5.99) | 169.9 | (9.16) | 170.1 | (8.43) |
| mean (SD), | | | | | | |
| cm | | | | | | |
| Weight, | 70.2 | (10.48) | 73.9 | (12.70) | 73.0 | (12.18) |
| mean (SD), | | | | | | |
| kg | | | | | | |
| BMI, mean (SD), | 24.0 | (2.53) | 25.3 | (3.16) | 25.1 | (3.06) |
| kg/m² | | | | | | |

A prior study of soticlestat dosing in dogs guided the safety margin of minimal and maximal single dose selection for human subjects. An initial dosage of 10 mg provided a 44-fold safety margin relative to the maximum safe dosage in dogs. Subjects were administered an oral solution of soticlestat in the morning after a 10-hour fast. Six subjects from 36 total were randomized into assigned soticlestat doses of 15, 50, 200, 600, 900 or 1350 mg. Each dose trial contained two placebo subjects. Ascending doses were tested in 2 sentinel subjects at a time to determine safety and tolerability before including the full cohort. Soticlestat and its metabolite (M-I) concentration were measured in plasma obtained from a 4 ml blood sample collected 30 minutes pre-dose on day 1 and at 0.25, 0.5, 1, 1.5, 2, 3, 4, 6, 8, 10, 12, 16, 24, 36, 48, 72, and 96 hours post-dose.

Urine samples were collected during the 12 hours pre-dose, and 0 to 6, 6 to 12, 12 to 24, 24 to 48, 48 to 72, and 72 to 96 hour intervals post-dose to determine soticlestat and M-I renal elimination quantity. Plasma and urine concentrations of soticlestat and M-I were measured by high performance liquid chromatography with tandem mass spectrometry detection (HPLC-MS/MS). The analytes and internal standards were extracted by solid phase extraction or by dilution. Assays were validated with a lower limit of quantitation of 1.00 ng/ml (LLOQ) to an upper limit of 2000 ng/mL for both soticlestat and M-I standards. Multiple analyses of pooled samples (n=6) verified intra- and inter-assay accuracy with an acceptance criteria of within 15% (CV≤15%) or 20% (CV≤20%) for the LLOQ. PK parameters were analyzed from plasma and urine concentration time data using standard non-compartmental analysis methods with Phoenix WinNonlin™ version 6.3 (Certara, Princeton, N.J. USA). All statistical analyses were performed using SAS software version 9.2 (SAS Institute Inc., Cary, N.C. USA).

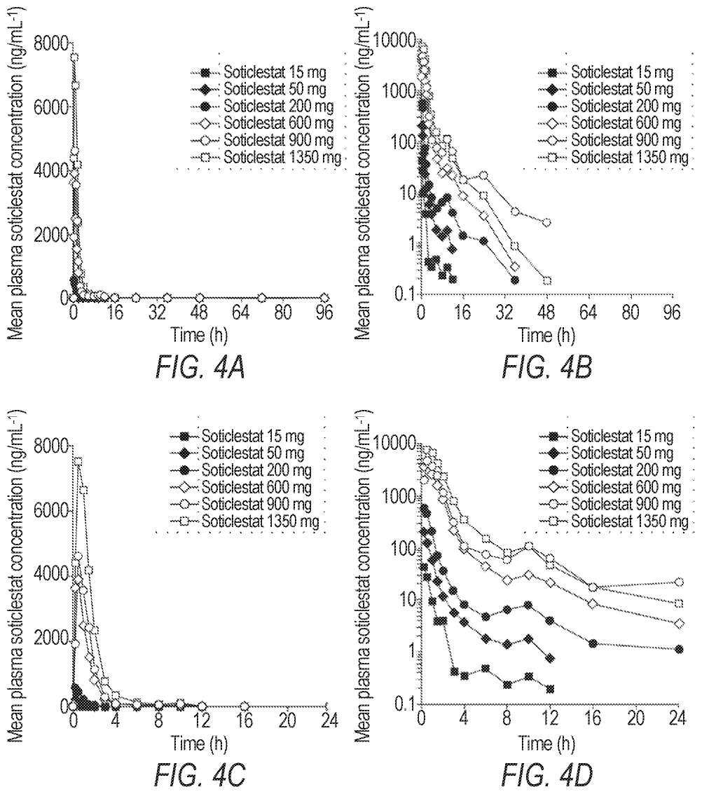

Single doses ranging from 15 mg to 1350 mg appeared well tolerated by the subjects. A total of five (41.7%) of the 12 subjects receiving placebo reported 13 treatment emergent adverse events (TEAEs), whereas 14 (38.9%) of the 36 receiving soticlestat reported 29 TEAEs. The TEAEs in soticlestat treated subjects did not appear to be dose related. The most frequently reported TEAEs were headache (placebo n=2; soticlestat, n=3), nausea (soticlestat, n=2), and dermatitis at the ECG electrode site (soticlestat, n=2). No abnormalities in clinical laboratory tests, ECGs, vitals, physical examinations, or visual function were observed in soticlestat treated subjects during the study. Table 6 shows a summary of PK parameter data obtained from the subject's plasma and urine. The mean values in table 6 with the superscript “a” were derived from n=3 subjects and values with superscript “b” were derived from n=4 subjects. FIG. 4 shows the mean plasma soticlestat concentration in subjects at different time points.

The maximum plasma concentration (Cₘₐₓ) ranged from 43.5 ng/ml for the 15 mg dose to 7950 ng/ml for the 1350 mg dose. The time to Cₘₐₓ (Tₘₐₓ) was between 0.25 hours and 0.52 hours for all soticlestat doses. A second smaller peak was observed at approximately 10 hours post-dose for all doses (see log-linear 24 hr, FIG. 4). The mean area under the curve from zero to infinity (AUC_{∞}) ranged from 23.3 (28.8) ng×h/mL for the 15 mg dose to 13500 (42.6) ng×h/mL for the 1350 mg dose. The mean area (CV %) under the curve from zero to the time of the last quantifiable concentration (AUC₍₀₋ₜ₎ produced similar results as AUC_{∞}, 33.3 (86.9) ng×h/mL for the 15 mg dose and 13500 (42.8) ng×h/mL for the 1350 mg dose. The mean terminal elimination half-life (t1/2) for soticlestat ranged from 0.820 hours to 7.16 hours. The mean apparent plasma clearance of drug after extravascular administration (CL/F) for soticlestat decreased with increased dosage, from 689 L/h (33.9) for the 15 mg dose to 112 L/h (34.7) for the 1350 mg dose. No dose-related trend was apparent in the mean volume of soticlestat distribution during the terminal phase after extravascular administration (V2/F). The negligible fraction of the soticlestat dose excreted in urine (fₑ%) and the renal clearance (CLR) indicates that renal elimination is not a significant means of soticlestat disposal.

The estimated slopes between the log-transformed soticlestat PK parameters and the log-transformed soticlestat failed to show dose proportionality. For instance, the plasma soticlestat concentration increased disproportionally to increasing dosage. Changes in drug absorption with increasing dosage could produce the nonlinear PK profile. The slope values are 1.22 (1.11, 1.32), 1.38 (1.28, 1.48) and 1.42 (1.31, 1.53) for Cₘₐₓ, AUC₍₀₋ₜ₎ and AUC_{∞}, respectively.

TABLE 6
Summary of plasma and urine pharmacokinetic parameters of soticlestat after administration of a single oral solution dose

| | Soticlestat | | Soticlestat | | Soticlestat | | Soticlestat | | Soticlestat | | Soticlestat | |
|---|---|---|---|---|---|---|---|---|---|---|---|---|
| | 15 mg | | 50 mg | | 200 mg | | 600 mg | | 900 mg | | 1350 mg | |
| | (n = 6) | | (n = 6) | | (n = 6) | | (n = 6) | | (n = 6) | | (n = 6) | |
| Soticlestat | | | | | | | | | | | | |
| plasma PK | | | | | | | | | | | | |
| parameters | | | | | | | | | | | | |
| Cₘₐₓ, mean | 43.5 | (94.7) | 210 | (72.8) | 571 | (45.1) | 4552 | (56.5) | 5080 | (35.5) | 7953 | (27.0) |
| (% CV), | | | | | | | | | | | | |
| ng/mL | | | | | | | | | | | | |
| Tₘₐₓ, median | 0.25 | (0.25-0.25) | 0.25 | (0.25-0.50) | 0.25 | (0.23-0.50) | 0.52 | (0.25-1.00) | 0.50 | (0.50-1.48) | 0.50 | (0.50-1.00) |
| (range), h | | | | | | | | | | | | |
| AUC(0-t), | 33.3 | (86.9) | 169 | (52.4) | 563 | (35.0) | 5790 | (59.2) | 7620 | (25.3) | 13 500 | (42.8) |
| mean (% | | | | | | | | | | | | |
| CV), ng · | | | | | | | | | | | | |
| h/mL | | | | | | | | | | | | |
| AUC_{∞}, mean | 23.3 | (28.8)^{a} | 172 | (62.1)^{b} | 621 | (38.7)^{a} | 5810 | (59.0) | 7660 | (25.6) | 13 500 | (42.6) |
| (% CV), ng · | | | | | | | | | | | | |
| h/mL | | | | | | | | | | | | |
| t_{1/2z}, mean | 0.820 | (82.8)^{a} | 2.99 | (68.8)^{b} | 4.39 | (43.3)^{a} | 4.77 | (53.8) | 7.16 | (34.3) | 5.31 | (21.3) |
| (% CV), h | | | | | | | | | | | | |
| V_{z}/F, mean | 732 | (62.3)^{a} | 1500 | (49.6)^{b} | 2240 | (50.1)^{a} | 993 | (69.7) | 1230 | (26.3) | 852 | (34.0) |
| (% CV), L | | | | | | | | | | | | |
| CL/F, mean | 689 | (33.9)^{a} | 549 | (112.4)^{b} | 355 | (37.4)^{a} | 145 | (64.3) | 124 | (25.4) | 112 | (34.7) |
| (% CV), L/h | | | | | | | | | | | | |
| Soticlestat | | | | | | | | | | | | |
| urine PK | | | | | | | | | | | | |
| parameters | | | | | | | | | | | | |
| CL_{R}, mean | 0.280 | (33.3) | 0.230 | (35.4) | 0.360 | (27.0) | 0.320 | (39.1) | 0.440 | (38.0) | 0.620 | (27.5) |
| (% CV), L/h | | | | | | | | | | | | |
| fₑ (%), mean | 0.0500 | (39.2) | 0.0700 | (46.6) | 0.100 | (27.4) | 0.360 | (91.8) | 0.400 | (57.8) | 0.600 | (42.2) |
| (% CV) | | | | | | | | | | | | |

Table 7 shows a summary of plasma and urine PK parameters for the soticlestat N-oxide metabolite (M-I). Mean plasma M-I concentrations increased with increasing soticlestat dose with Cₘₐₓ reached approximately 0.38-1.25 hours after soticlestat administration, followed by a relatively fast decline as indicated by t1/2z. The metabolic ratio (MR) decreased with increasing soticlestat dosage. Renal clearance (CLR) of M-I was higher than that of soticlestat at 3.23-4.76 L/h compared to 0.230-0.620 L/h. The CLR values of M-I were less proportional to dosage, indicating a saturation of soticlestat metabolic flux. From the urine soticlestat and M-I values, soticlestat appears to be predominantly metabolized by the liver and excreted as M-I in the urine. The mean values in table 4 with the superscript “a” were derived from n=5 subjects, superscript “c” from n=3 subjects, and superscript “d” from n=4 subjects.

TABLE 7
Summary of plasma and urine PK parameters of the soticlestat metabolite
M-I after administration of a single oral dose in healthy subjects

| | Soticlestat | Soticlestat | Soticlestat | Soticlestat | Soticlestat | Soticlestat |
|---|---|---|---|---|---|---|
| | 15 mg | 50 mg | 200 mg | 600 mg | 900 mg | 1350 mg |
| | (n = 6) | (n = 6) | (n = 6) | (n = 6) | (n = 6) | (n = 6) |
| M-I | | | | | | |
| plasma PK | | | | | | |
| parameters | | | | | | |
| Cₘₐₓ, | 19.3 | 56.4 | 151 | 542 | 833 | 900 |
| mean | (34.4) | (66.1) | (35.9) | (23.6) | (13.2) | (19.2) |
| (% CV), | | | | | | |
| ng/mL | | | | | | |
| Tₘₐₓ, | 0.50 | 0.38 | 0.50 | 0.53 | 1.00 | 1.25 |
| median | (0.25-1.98) | (0.25-0.50) | (0.48-0.53) | (0.50-1.00) | (0.50-1.50) | (0.98-1.50) |
| (range), h | | | | | | |
| AUC₍₀₋ₜ₎, | 39.1 | 114 | 293 | 1490 | 2710 | 3110 |
| mean | (43.5) | (55.1) | (26.5) | (31.8) | (17.1) | (18.3) |
| (% CV), | | | | | | |
| ng × h/mL | | | | | | |
| AUC_{∞}, | 45.8 | 118 | 299 | 1500 | 2780 | 3120 |
| mean | (37) | (53.6) | (25.9) | (31.7) | (18.0) | (18.2) |
| (% CV), | | | | | | |
| ng × h/mL | | | | | | |
| t1/2z, | 1.59 | 1.44 | 2.92 | 3.64 | 7.82 | 4.45 |
| mean | (29.2)^{a} | (10.8) | (32.3) | (45.7) | (52.6)^{a} | (47.7) |
| (% CV), h | | | | | | |
| MR (based | 1.84 | 0.750 | 0.580 | 0.300 | 0.340 | 0.240 |
| on | (42.9)^{c} | (30.0)^{d} | (24.4)^{c} | (37.6) | (22.8)^{a} | (32.6) |
| AUC_{∞})^{b} | | | | | | |
| mean | | | | | | |
| (% CV) | | | | | | |
| M-I urine | | | | | | |
| PK | | | | | | |
| parameters | | | | | | |
| CL_{R}, mean | 3.66 | 3.23 | 3.87 | 3.51 | 3.52 | 4.76 |
| (% CV), | (32.4) | (23.4) | (24.9) | (41.8) | (14.0) | (14.8) |
| L/h | | | | | | |

### Example 3. Comparison of Soticlestat Bioavailability in Solution Versus Tablet Formulation

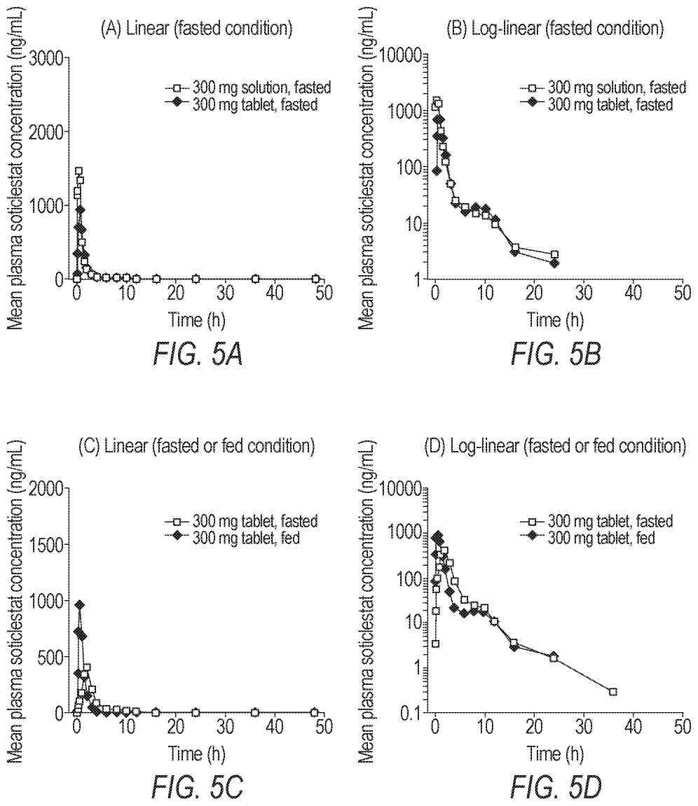

The bioavailability of soticlestat in solution versus tablet formulation was tested using the same PK data collection procedures as described in example 2. The graphs in FIG. 5A (linear) and 5B (log-linear) show that for a single 300 mg soticlestat dose, the oral solution and tablet soticlestat formulation produce similar plasma concentration-time profiles. Plasma soticlestat concentrations for the tablet formulation were only slightly lower than for the oral solution at 16 and 24 hours post-dose and there was no difference in t1/2z. Similar to the oral solution, approximately 10 hours after tablet ingestion, a secondary peak in plasma soticlestat level occurred. As shown in FIGS. 5A and 5B and table 9, ingestion of soticlestat in tablet form resulted in a 36.9% lower Cₘₐₓ compared with the oral solution. However, AUC_{∞} was only 15.8% lower. Ingestion of soticlestat tablet also delayed the median Tₘₐₓ to 0.53 hours compared to 0.35 hours for the oral solution.

### Example 4. Bioavailability of Soticlestat in Tablet Form in Fed Vs. Fasted Condition Compared with Oral Solution in Fasted Condition

A food-effect study measured the bioavailability of soticlestat in the fed state versus the fasting state. Nine subjects were randomized to three groups (A,B, and C) in an open-label cross-over study of 300 mg soticlestat bioavailability after ingesting tablet and solution formulations during fasted or fed condition. Group A and B treatment both consisted of swallowing a 300 mg soticlestat tablet, however group A was dosed 30 minutes after ingesting a high fat meal, while group B was dosed after a 10-hour fast. Group C treatment consisted of taking 300 mg soticlestat in an oral solution after a 10-hour fast. Table 8 shows the demographics for the nine enrolled subjects. Aside from differences in a few selected time points, PK data collection procedures and analyses are similar to example 2. The graph in FIG. 5C (linear) and 5D (log-linear) and the values in table 9 show that the mean soticlestat Cₘₐₓ plasma concentration for tablets under the fed condition decreased by 59.7% compared to the fasted condition. However, after reaching Cₘₐₓ and up to 10 hours post-dose, the mean soticlestat plasma concentration under the fed condition was slightly higher than the concentration under the fasting condition.

TABLE 8
Subject demographics and baseline characteristics
for food effect study

| | rBA/FE | |
|---|---|---|
| Characteristic | Total (n = 9) | |
| Age, mean (SD), years | 36.7 | (8.00) |
| Sex, n (%) | | |
| Male | 7 | (77.8) |
| Female | 2 | (22.2) |
| Race, n (%) | | |
| White | 3 | (33.3) |
| Black or African American | 5 | (55.6) |
| Multiracial | | |
| Ethnicity, n (%) | 1 | (11.1) |
| Hispanic or Latino | 2 | (22.2) |
| Non-Hispanic or Latino | 7 | (77.8) |
| Height, mean (SD), cm | 171.1 | (3.44) |
| Weight, mean (SD) kg | 77.6 | (9.04) |
| BMI, mean (SD) kg/m⁻² | 26.5 | (2.43) |

A transient sustained plasma soticlestat concentration was observed approximately 10 hours post-dose under both fed and fasting conditions but was more apparent under fasting conditions. When soticlestat was administered as an oral tablet under fed conditions, the AUC_{∞} was only 10.6% lower than that under fasting conditions. Compared with fasting conditions, the median Tₘₐₓ under the fed condition was delayed by approximately 1.5 hours. After a single-dose 300 mg soticlestat tablet, soticlestat t1/2z mean values were similar under fasting and fed conditions. Table 9 shows soticlestat Cₘₐₓ, AUC₍₀₋₀₎, and (AUC_{∞}) their 90% confidence interval for the point estimate.

TABLE 9
Plasma PK parameters after administration of a single soticlestat 300 mg dose (oral
tablet vs oral solution [fasted], and oral tablet [fed] vs oral tablet [fasted]

| | Tablet vs solution formulation | | | | | Food effect | | | | |
|---|---|---|---|---|---|---|---|---|---|---|
| | LS mean (natural logarithmic scale) | | | | | LS mean (natural logarithmic scale) | | | | |
| | | | Regimen | | | Regimen | | | | |
| | Regimen B: | | C: | | Point | A: | | Regimen | | Point |
| | Tablet, | | Solution, | | estimate | Tablet, | | B: Tablet, | | estimate |
| | fasted | | fasted | | (90% | fed | | fasted | | (90% |
| Parameter | n | test | n | reference | CI)* | n | (test) | n | reference | CI)* |
| Cₘₐₓ | 9 | 6.903 | 9 | 7.363 | 0.631 | 9 | 5.993 | 9 | 6.903 | 0.403 |
| ng/mL | | | | | (0.411, | | | | | (0.262, |
| | | | | | 0969) | | | | | 0.618) |
| AUC_{(0-t),} | 9 | 7.139 | 9 | 7.301 | 0.850 | 9 | 7.020 | 9 | 7.139 | 0.889 |
| ng × h/mL | | | | | (0.706, | | | | | (0.738, |
| | | | | | 1.024) | | | | | 1.070) |
| AUC_{∞}, | 8 | 7.144 | 8 | 7.315 | 0.842 | 9 | 7.031 | 8 | 7.144 | 0.894 |
| ng × h/mL | | | | | (0.676, | | | | | (0.726, |
| | | | | | 1.050) | | | | | 1.100) |

### Example 5. Pharmacodynamics of Soticlestat

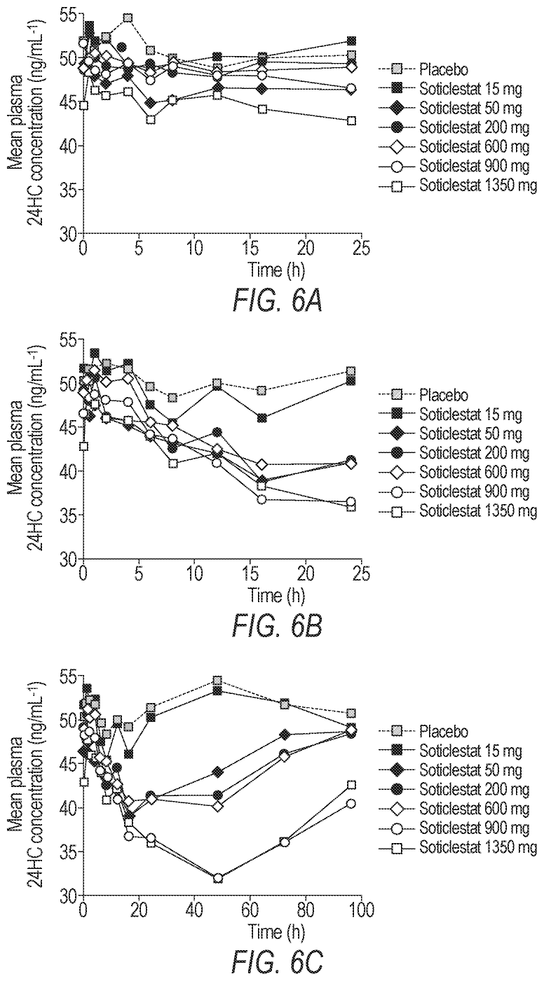

The pharmacodynamics of soticlestat were obtained from the same subjects studied in example 2. This study measured the plasma concentration-time profiles of 24HC at baseline and after administration of a single soticlestat dose at a range of dosage concentrations. The concentration of 24HC was measured by obtaining an 8 ml blood sample collected 30 minutes pre-dose time and 0.5, 1, 2, 4, 6, 8, 12, and 16 hours prior to dosing day, and then 30 minutes pre-dose time and at times 0.5, 1, 2, 4, 6, 8, 12, 16, 24, 48, 72, and 96 hours post-dose. Plasma 24HC levels were measured by incubating plasma in sodium hydroxide and methanol and then performing liquid-liquid extraction and HPLC-MS/MS multiple reaction monitoring. The validation range for the 24HC analyte and an internal standard had a LLOQ of 2.00 ng/mL and an upper limit of 100 ng/mL. Assay quality control measures, parameter analyses, and statistical analyses were performed as described in example 3. FIG. 6 shows mean plasma 24HC concentration across time for different soticlestat concentrations. FIG. 6A shows circadian fluctuation of 24HC levels 24 hours pre-dose. FIG. 6B shows 24HC levels after a single dose of soticlestat at different concentrations over 24 hours. Subjects treated with 900 mg soticlestat showed the maximum 24HC decrease of approximately 23% at 16 hours post-dose. Estimates of the area under the effect-time curve from zero to 24 hours and 96 hours, (AUEC₂₄) and (AUEC₉₆), and the effect at 24 hours (E24) showed that AUEC₉₆ decreased by approximately 28.7% compared to placebo. FIG. 6C shows 24HC concentration after dose time extended to 96 hours. All dosage groups returned to 24HC baseline values by 96 hours.

### Example 6. Multiple Rising Dose Study of Soticlestat to Study Safety, Tolerability, and PK Profiles of Soticlestat and its Metabolite M-I

This study administered soticlestat to healthy male and female adults in an oral solution at escalating daily dose levels. Table 10 shows the mean demographics and baseline anthropometric measurements for the 40 subjects enrolled in the study. Eight subjects were randomly assigned to one of five cohorts, with two subjects in each cohort receiving a placebo. Single daily dosing occurred in the morning after a minimum fast of 8 hours and subjects continued fasting until 2 hours post-dose. The study began with cohort 1 and 2 receiving a single oral dose of 100 mg and 300 mg of soticlestat for 14 days, respectively. After a full review of the safety data from cohorts 1 and 2, including vitals, physical examination, BMI, adverse events, and clinical laboratory tests, cohorts 3 and 4 were approved to receive increased dosages of 300 mg twice daily (BID) and 600 mg once daily (QD), respectively. After subjects in cohorts 3 and 4 experienced adverse neurological and psychiatric events, a soticlestat dosage of 400 mg was selected for cohort 5.

TABLE 10
Summary of subject demographics and baseline characteristics in rising dose study

| | | Soticlestat dose | | | | | |
|---|---|---|---|---|---|---|---|
| | | 100 mg | 300 mg | 400 mg | 300 mg | 600 mg | |
| | Placebo, | QD, | QD, | QD, | BID, | QD, | Total, |
| Characteristic | n = 10 | n = 6 | n = 6 | n = 6 | n = 6 | n = 6 | N = 40 |
| Age, mean, | 34.8 | 33.8 | 33.7 | 37.7 | 26.7 | 40.3 | 34.5 |
| years (SD) | (8.51) | (4.62) | (10.71) | (13.08) | (4.50) | (11.55) | (9.60) |
| Sex, n (%) | | | | | | | |
| Male | 7 | 5 | 3 | 4 | 5 | 6 | 30 |
| | (70.0) | (83.3) | (50.0) | (66.7) | (83.2) | (100.0) | (75.0) |
| Female | 3 | 1 | 3 | 2 | 1 | 0 | 10 |
| | (30.0) | (16.7) | (50.0) | (33.3) | (16.7) | | (25.0) |
| Ethnicity, n (%) | | | | | | | |
| Hispanic or | 5 | 0 | 4 | 5 | 4 | 1 | 19 |
| Latino | (50.0) | | (66.7) | (83.3) | (66.7) | (16.7) | (47.5) |
| Non-Hispanic | 5 | 6 | 2 | 1 | 2 | 5 | 21 |
| or non-Latino | (50.0) | (100.0) | (33.3) | (16.7) | (33.3) | (83.3) | (52.5) |
| Race, n (%) | | | | | | | |
| White | 7 | 5 | 4 | 5 | 5 | 5 | 31 |
| | (70.0) | (83.3) | (66.7) | (83.3) | (83.3) | (83.3) | (77.5) |
| Black or African | 3 | 1 | 2 | 1 | 1 | 1 | 9 |
| American | (30.0) | (16.7) | (33.3) | (16.7) | (16.7) | (16.7) | (22.5) |
| Height, mean, | 172.7 | 175.5 | 165.5 | 167.5 | 174.8 | 175.7 | 172.0 |
| cm (SD) | (10.02) | (5.65) | (6.16) | (7.29) | (8.57) | (5.43) | (8.19) |
| Weight, mean, | 78.90 | 79.37 | 67.68 | 75.28 | 73.55 | 78.25 | 75.85 |
| kg (SD) | (13.136) | (8.586) | (10.860) | (12.367) | (6.227) | (7.936) | (10.647) |
| BMI, mean, | 26.30 | 25.76 | 24.66 | 26.67 | 24.16 | 25.31 | 25.56 |
| kg/m² (SD) | (2.756) | (2.386) | (3.476) | (2.971) | (2.629) | (1.491) | (2.666) |

Blood samples were obtained for soticlestat and M-I PK data on days 1 and 14 at −30 minutes pre-dose and at 10, 15, 20, and 30 minutes, and post-dose at 1, 1.5, 2, 3, 4, 6, 8, 10, 12, 16, and 24 hours. All cohorts had additional blood samples taken 30 minutes pre-dose on days 7, 11, 12, and 13. Cohort 5 had additional blood samples taken for PK data on day 7 at post-dose times 15 and 30 minutes, and 1, 2, 4, and 8 hours. The PK parameter analysis was performed as described in example 2. Plasma and urine soticlestat and M-I concentrations were measured and validated as described in example 2. A power model estimated the dose proportionality for Cₘₐₓ and AUC_{∞} on day 1 and day 14 for cohorts receiving once daily dosing. An analysis of variance (ANOVA) assessed the variation in time dependency for Cₘₐₓ and AUC_{∞} on day 1 and day 14 for cohorts receiving once daily dosing.

Pre-dose urine samples were collected for PK analysis for 12 hours pre-dose of day 1 and for 2 hours pre-dose of day 14. Post-dose urine samples were collected at intervals of 0-6, 6-12, and 12-24 hours.

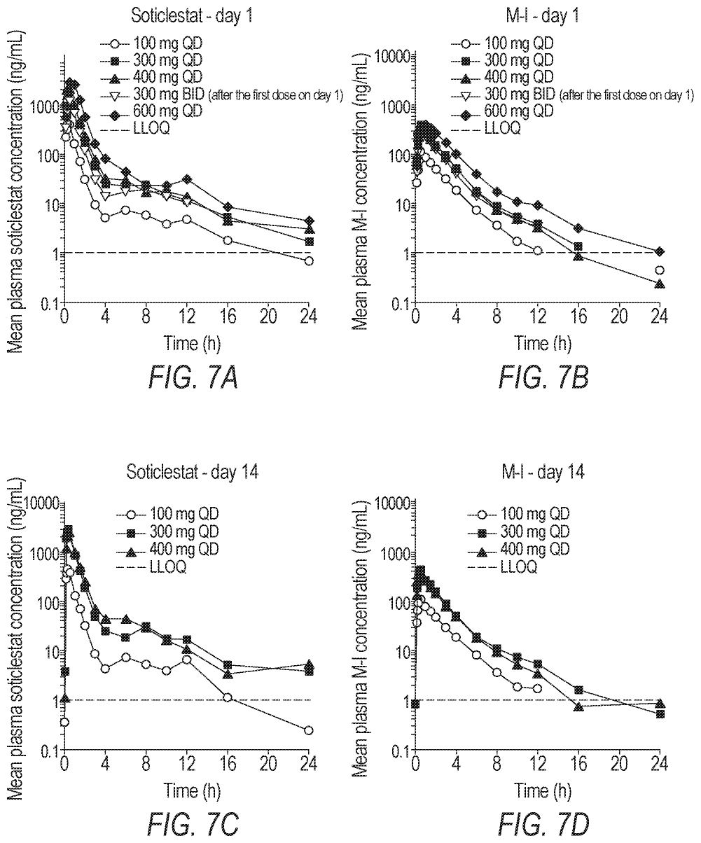

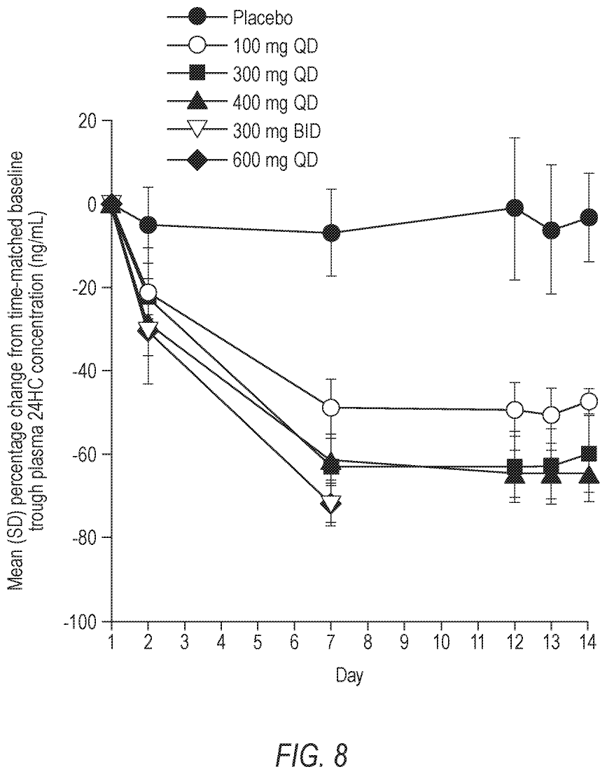

Table 11 and FIGS. 7A and 7B shows soticlestat PK data for cohorts receiving single and multiple doses. Soticlestat Cₘₐₓ from a 6-fold dose range (100-600 mg) ranged from 6.55 to 9.35 fold, respectively. The time to reach Cₘₐₓ, (Tₘₐₓ) was rapid and ranged from 0.33 to 0.5 hours after single or multiple dose regimens. The 600 mg cohort are not included in the multiple dose analysis since their treatment course terminated after day 10.

FIG. 7A shows soticlestat Cₘₐₓ and AUC₂₄ over the 4-fold dose range (100 to 400 mg) ranged from 6.08-fold to 6.12-fold on day 1, respectively. The dosage amount did not affect the mean terminal half-life (t1/2z) of soticlestat. There was no apparent difference in exposure accumulation from day 1 to day 14 for QD 100 mg and 400 mg. However, soticlestat Cₘₐₓ and AUC₂₄ at day 14 for the 300 mg QD cohort, shown in FIG. 7C, had an accumulated exposure of approximately 1.74-fold and 1.42-fold, respectively. Plasma soticlestat was higher at zero hour on day 14 in the 300 mg soticlestat QD cohort. As shown in example 2 table 6, soticlestat renal elimination is minimal.

Data in Table 11 and FIGS. 7B and 7D shows that Cₘₐₓ of M-I increased higher than the n-fold soticlestat dose increase. From 100 to 300 mg soticlestat, M-I increased 4.31 on day 1 to 6.44 on day 14. From 100 to 400 mg soticlestat, M-I increased 5.62 and on day 1 to 6.09 on day 14. The time of peak M-I in plasma (Tₘₐₓ) occurred between 0.5-1.0 hours post-dose, which is shortly after the soticlestat Tₘₐₓ.

TABLE 11
Estimates of pharmacokinetic parameters of soticlestat and M-I
after single (day 1) and multiple (day 14) doses of soticlestat

| Soticlestat dose | | | | | | | |
|---|---|---|---|---|---|---|---|
| 100 | | 300 | | 400 | | 300 | 600 |
| mg | | mg | | mg | | mg | mg |
| QD | | QD | | QD | | BID^{a, b} | QD^{a} |
| Day 1, | Day 14, | Day 1, | Day 14, | Day 1, | Day 14, | Day 1, | Day 1, |
| n = 6 | n = 6 | n = 6 | n = 6 | n = 6 | n = 6 | n = 6 | n = 6 |

Soticlestat,
plasma

| | | | | | | | | |
|---|---|---|---|---|---|---|---|---|
| Cₘₐₓ, mean, | 468 | 481 | 2020 | 3100 | 2630 | 2930 | 1330 | 3060 |
| ng/mL | (50.0) | (25.3) | (61.7) | (41.1) | (50.6) | (40.3) | (32.5) | (70.2) |
| (CV %) | | | | | | | | |
| Tₘₐₓ, | 0.42 | 0.42 | 0.42 | 0.33 | 0.33 | 0.33 | 0.44 | 0.50 |
| median, h | (0.33- | (0.25- | (0.33- | (0.17- | (0.25- | (0.33- | (0.33- | (0.50- |
| (range) | 0.53) | 0.50) | 1.05) | 0.33) | 0.50) | 0.50) | 1.00) | 1.00) |
| AUC_{∞}, | 451 | 509 | 2040 | 2840 | 2520 | 3090 | 1510 | 4220 |
| mean, | (36.7)^{c} | (24.8)^{c} | (55.3) | (40.1)^{d} | (48.1) | (32.8)^{d} | (48.0)^{e} | (59.4) |
| ng × h/mL | | | | | | | | |
| (CV %) | | | | | | | | |
| AUC₂₄ | 446 | 458 | 2030 | 2690 | 2500 | 2800 | 1460 | 4180 |
| mean, | (37.1)^{c} | (28.4) | (55.7) | (40.0) | (48.8) | (40.3) | (49.7)^{e} | (59.6) |
| ng × h/mL | | | | | | | | |
| (CV %) | | | | | | | | |
| t½z, mean, | 3.91 | 3.83 | 4.44 | 4.22 | 4.67 | 3.66 | 3.49 | 4.83 |
| h (CV %) | (20.7)^{c} | (14.2)^{c} | (19.8) | (23.4)^{d} | (38.1) | (42.4)^{d} | (59.2)^{e} | (20.9) |
| CL/F, | 248 | 234 | 185 | 130 | 187 | 166 | 232 | 194 |
| mean, L/h | (40.4)^{c} | (29.6) | (47.6) | (43.9) | (38.8) | (44.4) | (47.3)^{e} | (57.5) |
| (CV %) | | | | | | | | |
| V_{z}/F, | 1320 | 1140 | 1180 | 732 | 1330 | 713 | 1360 | 1320 |
| mean, L | (21.1)^{c} | (24.2)^{c} | (48.1) | (43.3)^{d} | (56.5) | (38.0)^{d} | (101)^{e} | (61.4) |
| (CV %) | | | | | | | | |

Soticlestat,
urine

| | | | | | | | | |
|---|---|---|---|---|---|---|---|---|
| Ae₂₄ mean, | 0.0900 | 0.0800 | 0.660 | 0.56 | 0.920 | 1.01 | 0.490 | 1.440 |
| mg (CV %) | (25.6) | (30.0) | (84.3) | (83.4) | (47.7) | (31.9) | (75.9) | (74.4) |
| CL_{R}, mean, | 0.220 | 0.200 | 0.300 | 0.220 | 0.370 | 0.380 | 0.350 | 0.340 |
| L/h (CV %) | (40.4) | (47.9) | (36.4) | (69.1) | (15.0) | (23.8) | (36.8) | (51.1) |
| fₑ, mean, % | 0.0900 | 0.0800 | 0.220 | 0.190 | 0.230 | 0.250 | 0.160 | 0.240 |
| (CV %) | (25.6) | (30.0) | (84.3) | (83.4) | (47.7) | (31.9) | (75.9) | (74.4) |

M-I, plasma

| | | | | | | | | |
|---|---|---|---|---|---|---|---|---|
| Cₘₐₓ, mean, | 105 | 106 | 398 | 449 | 369 | 383 | 302 | 407 |
| ng/mL | (16.7) | (23.5) | (35.2) | (33.9) | (28.3) | (24.8) | (18.8) | (17.4) |
| (CV %) | | | | | | | | |
| Tₘₐₓ, | 0.51 | 0.50 | 0.50 | 0.50 | 0.50 | 0.50 | 0.50 | 1.00 |
| median, h | (0.33- | (0.33- | (0.50- | (0.33- | (0.33- | (0.33- | (0.37- | (0.50- |
| (range) | 1.00) | 0.53) | 1.05) | 0.50) | 1.00) | 0.50) | 1.00) | 1.50) |
| AUC_{∞}, | 258 | 245 | 788 | 829 | 731 | 795 | 601 | 1180 |
| mean, | (18.6)^{d} | (19.8) | (35.3) | (41.9)^{d} | (31.8) | (27.6)^{d} | (29.4) | (23.8) |
| ng × h/mL | | | | | | | | |
| (CV %) | | | | | | | | |
| AUC_{τ},^{f} | 255 | 243 | 784 | 858 | 726 | 741 | 590 | 1170 |
| mean, | (18.5)^{d} | (19.7) | (35.5) | (37.3) | (32.1) | (31.8) | (29.2) | (23.7) |
| ng × h/mL | | | | | | | | |
| t_{1/2z}, mean, h | 2.34 | 2.32 | 3.15 | 2.50 | 3.43 | 2.42 | 2.40 | 3.88 |
| (CV %) | (23.3)^{d} | (5.3) | (44.4) | (28.5)^{d} | (53.1) | (21.2)^{d} | (15.8) | (32.1) |
| MR (based | 0.560 | 0.440 | 0.410 | 0.280 | 0.310 | 0.260 | 0.410 | 0.330 |
| on AUC_{∞}), | (40.9) | (17.7) | (22.8) | (14.9) | (35.1) | (39.6) | (17.3) | (47.0) |
| mean, | | | | | | | | |
| (CV %) | | | | | | | | |

M-I, urine

| | | | | | | | | |
|---|---|---|---|---|---|---|---|---|
| Aeₜ,^{g} mean, | 0.620 | 0.600 | 3.08 | 1.97 | 2.70 | 2.99 | 2.28 | 4.48 |
| mg (CV %) | (31.1) | (34.4) | (51.5) | (82.7) | (28.1) | (32.0) | (39.7) | (31.4) |
| CL_{R}, mean, | 2.55 | 2.45 | 3.83 | 2.42 | 3.88 | 4.10 | 3.87 | 3.81 |
| L/h (CV %) | (22.5) | (22.1) | (17.9) | (61.8) | (29.5) | (19.5) | (23.0) | (18.5) |

The AUC_{∞} and AUC₂₄ for M-I were comparable from day 1 to 14 after QD for 100, 300, and 400 mg. It was apparent that the mean metabolic ratio (MR), based on AUC_{∞}, decreased with increasing dosage and with the duration of exposure from day 1 to 14. For instance, the MR ranged from 0.56 to 0.31 after single doses ranging from 100 to 600 on day 1 to 0.44 to 0.26 after multiple doses ranging from 100 to 400 mg on day 14. Accordingly, the M-I t1/2z increased from 2.34 to 3.43 for 100 to 400 mg dosages. The renal clearance (CLR) of M-I did not changed in proportion to increased soticlestat dosage. Cohorts with the superscript “a” were administered 300 mg BID or 600 mg QD groups and discontinued dosing from day 11 to day 14. Therefore, PK parameters on day 14 were not available for the 300 mg BID and 600 mg QD groups. The soticlestat PK parameters were derived using plasma concentration data after the first dose on day 1 for the 300 mg BID cohort. The mean values in table 8 with superscript “c” were derived from n=4 subjects, superscript “d” n=5 subjects; superscript “e” n=3 subjects.

### Example 7. Pharmacodynamics of Soticlestat Dosing Over Multiple Days at a Range of Concentrations

This study measured the pharmacodynamics of soticlestat from the patients described in example 6. Plasma 24HC collection and measurement were as described in example 5, with the exception that the 30—minute pre-dose sampling occurred on day 1, 7, 12, 13 and 14, and the 24-hour sampling occurred on day 1 and 14. Assay quality control measures, parameter and statistical analyses were performed as described in example 5. FIG. 8 shows that plasma 24HC decreased in a soticlestat dose-dependent manner over multiple days, approaching the maximal decrease by day 7. Table 12 shows a dose-dependent decrease in percent change from baseline in 24HC area under the effect-time curve from 0 to 24 hours (AUEC₂₄) and at 24 hours (E24) on day 14. On day 14, AUEC₂₄ ranged from −46.8 to −62.7 after a single dose of 100 to 400 mg soticlestat. The data indicate that single oral doses of soticlestat solution from 100 to 400 mg lowers 24HC level. The dosage increase from 300 mg to 400 mg daily did not appreciably decrease 24HC levels further. Subjects in the 300 mg BID and 600 mg QD cohorts discontinued treatment after day 10 due to some subjects experiencing adverse effects.

TABLE 12
Summary of percent (%) change from baseline (day −1) in 24HC parameters
on day 1 and day 14 after single and multiple doses of soticlestat

| PD | | | | | | | | |
|---|---|---|---|---|---|---|---|---|
| parameters | Soticlestat dose | | | | | | | |
| (percent | 100 mg | | 300 mg | | 400 mg | | 300 mg | 600 mg |
| change | QD | | QD | | QD | | BID | QD |
| from | Day 1, | Day 14, | Day 1, | Day 14, | Day 1, | Day 14, | Day 1, | Day 1, |
| baseline) | n = 6^{a, b} | n = 6^{c, d, e} | n = 6^{a, b} | n = 6^{c, d, e} | n = 6^{a, b} | n = 6^{c, d, e} | n = 6^{a, b} | n = 6^{a, b} |
| AUEC₂₄. | −8.15 | −46.8 | −12.7 | −61.9 | −11.7 | −62.7 | −6.90 | −14.0 |
| mean, | (−45.4) | (−9.2) | (−36.5) | (−17.0) | (−34.5) | (−7.3) | (−75.9) | (−27.9) |
| h · ng/mL | | | | | | | | |
| (CV %) | | | | | | | | |
| E₂₄. mean, | −20.9 | −49.1 | −22.4 | −63.8 | −28.4 | −65.6 | −10.5 | −30.6 |
| ng/mL | (−49.0) | (−11.9) | (−19.5) | (−19.3) | (−28.1) | (−6.0) | (−63.8) | (−41.0) |
| (CV %) | | | | | | | | |

### Example 8. Soticlestat Safety in Healthy Subjects Taking Soticlestat for 14 Days

Soticlestat safety metrics were obtained from the patients described in example 6. The rising dose study tested the safety and tolerability of soticlestat dosages in healthy subjects over 14 days. Subjects were monitored during the study and a full review was conducted after each rising dose. Safety data monitoring included vitals, physical and visual examinations, electrocardiogram, BMI, and clinical laboratory tests. There was no clinically relevant findings. Table 13 summarizes the collected safety data. Percentages are rounded to one decimal place. A subject with two or more different adverse events (AE) within the same levels of the MedDRA term and treatment is counted only once at that level using the most related or severe incident. A TEAE is defined as an AE or SAE that occurs or gets worse after receiving the first dose of study drug and within 30 days after the last dose of study drug.

Forty-five treatment-emergent adverse events (TEAEs) were reported by 14 subjects (46.7%) taking soticlestat. Of the 45 TEAEs, 31 were considered related to soticlestat treatment. Most TEAEs (91%) were mild and transient. However, two subjects, one taking 600 mg soticlestat QD and the other taking 600 mg soticlestat BID, experienced severe acute psychosis and a mild state of confusion, respectively. The reported TEAEs were reversible and resolved without treatment. There were no serious adverse events (SAEs) reported. In summary, in this study, soticlestat was demonstrated to be safe and tolerated by healthy men and women up to 400 mg daily.

TABLE 13
Summary of treatment-emergent adverse events

| Number of | Placebo | 100 mg | 300 mg | 300 mg | 400 mg | 600 mg | | |
|---|---|---|---|---|---|---|---|---|
| subjects | QD, | QD, | QD, | BID, | QD, | QD, | total, | |
| (%) | n = 10 | n = 6 | n = 6 | n = 6 | n = 6 | n = 6 | n = 30 | |
| TEAEs | 4 (40.0) | 0 | 1 (16.7) | 5 (83.3) | 5 (83.3) | 3 (50.0) | 14 | (46.7) |
| Related | 2 (20.0) | 0 | 1 (16.7) | 5 (83.3) | 4 (66.7) | 3 (50.0) | 13 | (43.3) |
| Not | 2 (20.0) | 0 | 0 | 0 | 1 (16.7) | 0 | 1 | (3.3) |
| related | | | | | | | | |
| Mild | 4 (40.0) | 0 | 1 (16.7) | 5 (83.3) | 4 (66.7) | 2 (33.3) | 12 | (40.0) |
| Moderate | 0 | 0 | 0 | 0 | 1 (16.7) | 0 | 1 | (3.3) |
| Severe | 0 | 0 | 0 | 0 | 0 | 1 (16.7) | 1 | (3.3) |
| Leading to | 0 | 0 | 0 | 1 (16.7) | 0 | 1 (16.7) | 2 | (6.7) |

| | | | | | | | |
|---|---|---|---|---|---|---|---|
| halt | | | | | | | |
| SAEs | 0 | 0 | 0 | 0 | 0 | 0 | 0 |
| Deaths | 0 | 0 | 0 | 0 | 0 | 0 | 0 |

### Example 9. Safety and Tolerability of Soticlestat in Subjects with Developmental and/or Epileptic Encephalopathy

This trial tested soticlestat in adults ages 18-65 with developmental and/or epileptic encephalopathy and a mean of at least two bilateral motor seizures per month in the three months prior were enrolled in a 4-week baseline study. Only patients having at least one bilateral seizure during the baseline study were eligible to continue. Table 14 shows the patient demographics and diagnoses. The range of seizure types included tonic-clonic, drop seizures, tonic, atonic, bilateral clonic, myoclonic-atonic, myotonic-tonic-clonic, and focal seizures. Aside from seizure history, subjects were also enrolled based on an establish diagnosis of DEE, such as Lennox-Gastaut syndrome (LGS) and Dravet syndrome (DS), tuberous sclerosis complex, and a history of special education or an overall intellectual quotient of <70. Patients were excluded from the study if they had a clinically significant abnormal electrocardiogram (ECG) at screening, degenerative eye disease, or required mechanical respiration 3-months prior to screening. Subjects could continue taking 1-4 anti-seizure medicines (ASM), with the exception of stiripentol, during the study.

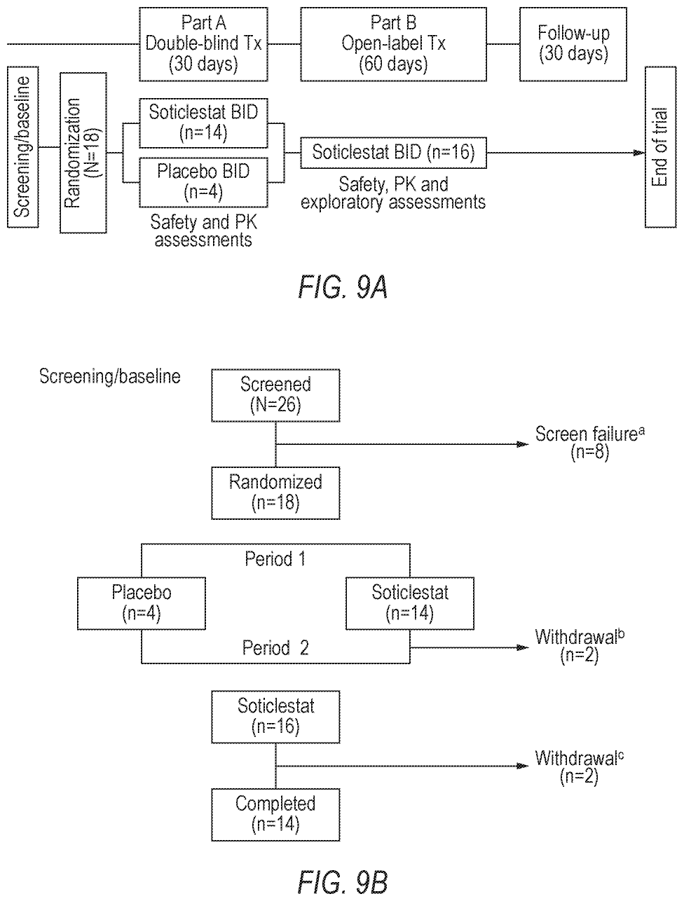

FIG. 9A depicts the study paradigm and FIG. 9B depicts the patient disposition. Part A consisted of a randomized, double blind, placebo controlled study with patients receiving soticlestat or placebo for 30 days. Part B consisted of an open-label 85-day extension study to ensure long-term safety and tolerability. The first 20 days of part A consisted of a tolerability titration phase, and thereafter a maintenance phase lasting from days 21-30.

TABLE 14
Patient demographics and type of developmental
and/or epileptic encephalopathy diagnoses

| | Placebo | | Soticlestat | | Total | |
|---|---|---|---|---|---|---|
| | (n = 4) | | (n = 14) | | (N = 18) | |
| Demographics | | | | | | |
| Age, years | 26.4 | (19, 39) | 28.7 | (20, 45) | 28.5 | (19, 45) |
| Median (min, max) | | | | | | |
| Sex, n (%) | | | | | | |
| Male | 4 | (100) | 10 | (71) | 14 | (78) |
| Female | — | | 4 | (29) | 4 | (22) |
| Race, n (%) | | | | | | |
| Caucasian | 3 | (75) | 13 | (93) | 16 | (89) |
| Black or African American | 1 | (25) | — | | 1 | (6) |
| Not reported | — | | 1 | (7) | 1 | (6) |
| Seizures at baseline, n per 28 days | | | | | | |
| Median (min, max) | 10.10 | (3.7, 49.5) | 33.75 | (3.4, 277.1) | — | |
| Diagnoses, n (%) | | | | | | |
| Lennox-Gastaut syndrome | 1 | (25) | 4 | (29) | 5 | (27.8) |
| Epileptic encephalopathy^{a} | 1 | (25) | 3 | (21.4) | 4 | (22.2) |

| | | | | | |
|---|---|---|---|---|---|
| Partial seizures with | — | 1 | (7) | 1 | (6) |
| Frontal lobe epilepsy | — | 1 | (7) | 1 | (6) |
| Tuberous sclerosis complex | — | 1 | (7) | 1 | (6) |
| Cerebral dysgenesis | — | 1 | (7) | 1 | (6) |
| Hypothalamic hamartoma | — | 1 | (7) | 1 | (6) |
| Dravet syndrome | — | 1 | (7) | 1 | (6) |

The safety and tolerability assessments included blood pressure, heart rate, respiratory rate, temperature, treatment-emergent adverse effects, clinical laboratory tests, electrocardiogram data, neurological and ophthalmic examination, and the Columbia-suicide severity rating scale (C-SSRS).

Patients were dosed 100 mg, 200 mg, or 300 mg BID. The dose titration pattern in part A is shown in table 15. Dosage (mg BID) in each interval reflects the final dose during that interval. Fields showing 0 mg in days 1-10, 11-20, and 21-30 intervals are patients who were assigned to the placebo group in Part A of the study.

TABLE 15
Dose adjustment pattern

Dosage (mg BID)

| | | | | | |
|---|---|---|---|---|---|
| Number of patients | Days | Days | Days | Days | Days |
| n (%) (n = 18) | 1-10 | 11-20 | 21-30 | 31-40 | 41-84 |

| | | | | | | |
|---|---|---|---|---|---|---|
| 4 | (22.2) | 0 | 0 | 0 | 200 | 300 |
| 1 | (5.6) | 100 | | | | |
| 1 | (5.6) | 100 | 100 | 100 | 200 | 300 |
| 1 | (5.6) | 100 | 200 | | | |
| 1 | (5.6) | 100 | 200 | 200 | 200 | 100 |
| 1 | (5.6) | 100 | 200 | 200 | 200 | 300 |
| 1 | (5.6) | 100 | 200 | 300 | 200 | 100 |
| 1 | (5.6) | 100 | 200 | 300 | 200 | 200 |
| 7 | (38.9) | 100 | 200 | 300 | 200 | 300 |

Table 16 shows the numbers of treatment-emergent adverse events (TEAEs) and serious adverse events (SAEs) for patients taking soticlestat or placebo. TEAEs were higher in the placebo group (100%) compared to the soticlestat group (71.4%). Part B had a similar frequency of TEAEs to the soticlestat arm in part A. In part A, the most frequent TEAE reported was transient dysarthria, which is a speech motor disorder. Other TEAEs reported were upper respiratory infection, lethargy, and headache. Two patients in part A and two patients in part B discontinued treatment due to TEAEs or SAEs. The first patient to discontinue treatment experienced gait disturbance and lethargy. The second patient to discontinue treatment experienced moderate lethargy. During part A, a third patient reported a severe seizure cluster but the event was considered unrelated to soticlestat. In part B, the same patient experienced two additional seizures clusters that appeared related to the drug, therefore the patient withdrew from the study. A fourth patient reported a serious seizure cluster, which appeared related to the soticlestat, thus the patient withdrew from the study. In addition to these four patients, a fifth patient experienced seizure clusters of moderate intensity, but they appeared be unrelated to the soticlestat so the patient remained in the study. In total, three patients reported five seizure related SAEs. Another patient reported intellectual impairment on day 22 and on day 87, but not at screening. There were no other clinically significant abnormalities identified during part A or part B. In addition, there were no deaths in either the soticlestat and placebo treatment arms

TABLE 16
Treatment-emergent adverse events and serious adverse events

| | |
|---|---|
| Part A | |
| Placebo | Part B |

| | | | |
|---|---|---|---|
| Frequency and severity | (n = 4) | Soticlestat (n = 14) | All (n = 16) |

Number of participants with at least one TEAE, n (%)

| | | | | | | |
|---|---|---|---|---|---|---|
| AEs | 4 | (100) | 10 | (71.4) | 11 | (68.8) |
| Mild AE | 4 | (100) | 7 | (50) | 4 | (25) |

| | | | | | |
|---|---|---|---|---|---|
| Moderate | — | 2 | (14.3) | 5 | (31.3) |
| Severe | — | 1 | (7.1) | 2 | (12.5) |
| AE-related withdrawal^{a} | — | 2 | (14.3) | 2 | (12.5) |
| SAEs^{b, c} | — | 1 | (7.1) | 3 | (18.8) |

Number of participants with most common (≥three participants overall [10%]) non-
serious AEs, n (%)^{d} AEs, n (%)^{d}

| Dysarthria | — | | 3 | (21) | — | |
|---|---|---|---|---|---|---|
| Fatigue | 1 | (25) | 2 | (14) | — | |
| Headache | 1 | (25) | 2 | (14) | — | |
| Insomnia | — | | — | | 3 | (19) |
| Lethargy | — | | 2 | (14) | 2 | (12.5) |
| Seizure | — | | — | | 3 | (19) |
| Upper respiratory | | | 2 | (14) | 1 | (6) |
| tract infection | | | | | | |

### Example 10. Pharmacokinetics of Soticlestat in Developmental and/or Epileptic Encephalopathy Patients

The pharmacokinetics of soticlestat was measured in the developmental and/or epileptic encephalopathy patients described in example 9. Blood samples were collected before and after the morning dose on days 1, 11, and 21, and before and after the morning dose on days 31, 41, and 85. Plasma soticlestat concentrations were measured as described in example 2. Patients dosed twice daily with 100, 200, or 300 mg soticlestat showed a soticlestat Cₘₐₓ steady state of 269.6, 639.8, and 975.3 ng/ml; a Ctrough of 10.5, 26, and 30.2 mg/mL; and a mean steady-state area under the curve of 562.5, 1437, and 2188 ng·h/mL. The mean clearance at 100, 200, and 300 mg BID soticlestat was 259.1, 195.8, and 190 L/h

### Example 11. Soticlestat Pharmacodynamics and Efficacy in Developmental and/or Epileptic Encephalopathy Patients

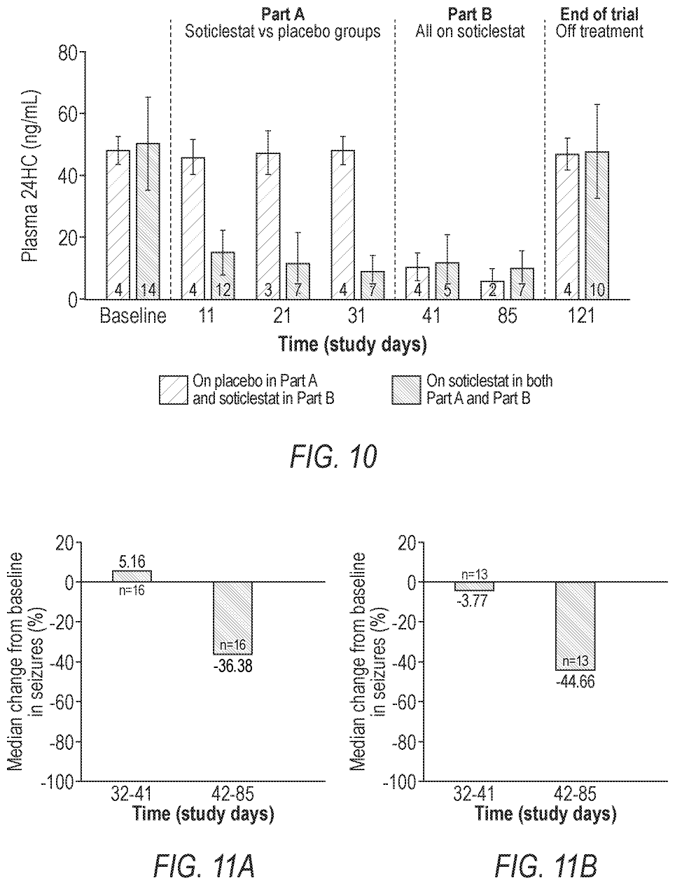

The pharmacodynamics of soticlestat was measured in the developmental and/or epileptic encephalopathy patients described in example 9. Blood sample collection and analyses for measuring 24HC was performed as previously described in example 5. FIG. 10 shows that plasma 24HC decreases in patients treated with soticlestat compared to the placebo treated group. The mean percent change from baseline was −69.76% at day 11 and −76.88% at day 21 for soticlestat versus −4.30% and −0.71% for placebo. The plasma concentration of 24HC continued to decrease to day 85, reaching −80.97%. Recovery to pre-dose 24HC baseline levels occurred by 36 days after the last soticlestat dose. The soticlestat lowering effect on 24HC appeared to plateau when soticlestat at steady-state (AUC₀₋ₜ) was greater than 800 ng×h/mL.

### Antiseizure Efficacy

Patients randomized to the soticlestat group (n=14) had more seizures than patients in the placebo group (n=4) in the 28 day period prior to treatment, with a median of 33.75 versus 10.10. In part A, the median percent change in seizures frequency from baseline for the soticlestat group was +16.71%, and ranged between −63% and +465%, and for the placebo group +22.16%, ranging between −73% to +142%. Three patients treated concurrently with the ASM perampanel had increased seizure activity from their baseline frequency (+107%, +465%, and +4%). Subtracting the three patients taking perampanel in part A resulted in a median percent change of seizure frequency from baseline of +7.54%.

In the part B, or the maintenance phase, the median percent change from baseline seizure frequency was −36.38% from baseline (n=16), with a range in median percent change from baseline seizure frequency from −100 to +398%. This percent change in baseline seizure frequency included the three patients taking perampanel, which increased +297%, +398%, and +175. FIG. 11B shows that excluding these three patients results in a median percent change from baseline seizure frequency of −44.66% (n=13). In the last 28 days of part B, the median percent change from baseline seizure frequency was −60.74%. The median seizure frequency for all completers, except those taking perampanel, was 25.60 per 28 days, which is less than the baseline frequency of 33.75 per 28 days. Two patients had 100% reduction in seizure frequency in their last month of treatment. In summary, soticlestat treatment appeared to reduced seizure frequency in developmental and/or epileptic encephalopathy patients in the open-label study.

### Example 12. Population Pharmacokinetic Modeling

Population pharmacokinetic (PK) modeling analysis was performed to define the relationship between dose and soticlestat exposure and define covariates with significant effects. Subject data for models included healthy subjects as well as pediatric and adult patients with epilepsy encephalopathies. Modeling data was derived from studies presented in examples 2, 4, 6, and 10, with the exception of data from the high-fat fed arm in example 4. More data was added from an open-label, positron emission tomography, phase 1 study wherein soticlestat CH24H binding was estimated based on its ability to displace ¹⁸ [F]MNI-792 occupancy from CH24H. The modeling analysis also included data from a randomized, double-blind, placebo-controlled phase 2 study called ELEKTRA, which evaluated the safety, tolerability, and efficacy of soticlestat in a study of approximately 126 pediatric patients with DEE. The starting dose was adjusted according to the patient's body weight (Table 17).

TABLE 17
Dosing schedule by weight in ELEKTRA patients

| Weight | Dose 1 | | Dose 2 | | Dose 3 | |
|---|---|---|---|---|---|---|
| (kg) | mg/day | No. tablets | mg/day | No. tablets | mg/day | No. tablets |
| 10-14 | 80 | 4 | 160 | 8 | 220 | 11 |
| 15-19 | 120 | 6 | 200 | 10 | 260 | 13 |
| 20-24 | 120 | 6 | 240 | 12 | 320 | 16 |
| 25-29 | 120 | 6 | 240 | 12 | 360 | 18 |
| 30-34 | 160 | 8 | 280 | 14 | 400 | 20 |
| 35-39 | 160 | 8 | 280 | 14 | 440 | 22 |
| 40-44 | 160 | 8 | 320 | 16 | 480 | 24 |

Altogether, this data allowed modeling the relationship between soticlestat dose and exposure and identified covariates (independent variables) significantly affecting PK.

The variables for the modeling study included subject variables (treatment formulation, dosage no., epileptic syndrome), treatment variables (dosage and time intervals), subject baseline demographic variables (sex, weight, height, age, body mass index, gender, ethnicity, and race), and baseline laboratory variables (aspartate aminotransferase, gamma glutamyl transferase, alkaline phosphatase, bilirubin, creatinine, creatinine clearance (CRCL), A1-AGLP, and eGFR. eGFR in mL/min/1.73m2 was estimated using the Shull formula [Shull, 1978]

$$eGFR = \frac{3.5 \times Age + 23.6}{Creatinine \times 0.011312}$$

Creatinine clearance (CRCL) in min/mL was estimated using the Cockcroft-Gault formula [Cockcroft, 1976]

$$CRCL = \frac{( 140 - Age ) \times Weight \times ( if Sex = Female then 1.04 else 1.23}{Creatinine}$$

In addition to the shared variables described above, Table 18 lists the groups of anti-epileptic co-medications some patients were taking during the study. Some anti-epileptic medications altered soticlestat bioavailability and either increased or reduced the CH24H enzyme inhibitory effect of soticlestat.

TABLE 18
Groups of anti-epileptic drugs taken
concomitantly in the phase 2 study

| | | | | Per- |
|---|---|---|---|---|
| Group | Variable | Drug Names | N | cent |
| Cannabidiol | AED_CANNA | “CANNABIDIOL” | 10 | 6.4 |
| Neutral 1 | AED_NEUT1 | “CLOBAZAM”, | 99 | 63.1 |
| Neutral 2 | AED_NEUT2 | “CLONAZEPAM”, | 82 | 52.2 |
| Stiripentol | AED_STIRI | “DIAZEPAM” | 27 | 17.2 |
| Strong | AED_{—} | “LACOSAMIDE”, | 20 | 12.7 |
| Inducer | STR_IND | | | |
| Topiramate | AED_TOPIR | “LAMOTRIGINE”, | 43 | 27.4 |
| Valproates | AED_VALPR | “LEVETIRACETAM”, | 102 | 65.0 |
| Weak | AED_WEAKI | “LORAZEPAM” | 51 | 32.5 |
| Inducer | | | | |

Table 19 summarizes the example nos. and clinical trial IDs, number of subjects, daily dose regimen, and number of observations and total doses. Tables 20-23 summarize the categorical and continuous covariate data used for PK modeling analysis from the entire population and for the ELEKTRA patient subset. The PK dataset also contained individual estimates of population PK parameters (IND_ALAG1, IND_KA, IND_V2, IND_Q, IND_V3, and IND_F1). The PK parameters are defined above in the definition section. The dataset analyzed included 69 single doses and 226 steady-state dosing events. A mixed effects PK population model strategy was tested using the computer program NONMEM. Aspects such as between and within subject variability, covariates, and structural aspects of the model were considered in a stepwise procedure, wherein models with additional parameters were compared to previous models. The models were accessed by the following goodness of fit (GOF) methods:
Standard goodness of fit plots
Biological plausibility of fixed and random effects of covariates
Numerical properties of model fitting procedure
Precision of the parameter estimates, as measured by the relative standard error (RES %=

100% ·standard error of the estimate/|parameter estimate|)
Shrinkage on between subject variability (RBV) and residual variability (RV) level (eta and epsilon shrinkage).

TABLE 19
PK population modeling dataset summary

| | | Daily Dose | Number of | Number of | Number |
|---|---|---|---|---|---|
| Example | Clinical Trial ID | number | Subjects | Observations | of Doses |
| 2 | NCT02201056 | single dose | 36 | 419 | 36 |
| 4 | NCT02906813 | single dose | 9 | 258 | 24 |
| 6 | NCT02539134 | multiple dose | 30 | 809 | 432 |
| n/a | NCT02497235 | Single dose | 11 | 47 | 11 |
| 10 | NCT03166215 | multiple dose | 17 | 119 | 2247 |
| 12 | NCT03650452 | multiple dose | 71 | 554 | 505 |
| | (ELEKTRA) | | | | |

TABLE 20
Categorical covariates for the entire PK modeling population

| Variable | Level | Count | Percent |
|---|---|---|---|
| Disease | 2001: EE | 17 | 10 |
| | 2002: Dravet S. | 26 | 15 |
| | 2002: Lennox-Gastaut S. | 45 | 26 |
| | Healthy Volunteer | 86 | 49 |
| Ethnicity | Hispanic or Latino | 36 | 21 |
| | Non-Hispanic or Latino | 138 | 79 |
| Freq. | BID | 77 | 44 |
| | QD | 97 | 56 |
| Race | Asian | 21 | 12 |
| | Black or African | 24 | 14 |
| | Multiracial | 2 | 1 |
| | Not Reported | 1 | 1 |
| | White | 126 | 72 |
| Sex | F | 51 | 29 |
| | M | 123 | 71 |

TABLE 21
Categorical covariate data from the ELEKTRA population

| Variable | Level | Count | Percent |
|---|---|---|---|
| Chinese | No | 51 | 72 |
| | Yes | 20 | 28 |
| Tablet Crushed | No | 49 | 69 |
| | Yes | 22 | 31 |
| Disease | 2002: Dravet S. | 26 | 37 |
| | 2002: Lennox-Gastaut S. | 45 | 63 |
| Ethnicity | Hispanic or Latino | 11 | 15 |
| | Non-Hispanic or Latino | 60 | 85 |
| G-Tube | No | 62 | 87 |
| | Yes | 9 | 13 |
| Race | Asian | 21 | 30 |
| | White | 50 | 70 |
| Sex | F | 22 | 31 |
| | M | 49 | 69 |

TABLE 22
Summary of continuous covariates from the entire PK population

| Statistic | Age | Weight | BMI | AST | ALT | CrCL | eGFR | Albumin | A1-AGLP | GGT | ALP |
|---|---|---|---|---|---|---|---|---|---|---|---|
| cc | (y) | (kg) | (kg/m2) | (U/L) | (U/L) | (mL/min) | (mL/min/1.73 m2) | (g/L) | (umol/L) | (U/L) | (U/L) |
| N | 174 | 174 | 174 | 174 | 174 | 174 | 174 | 174 | 92 (+82 NA) | 174 | 174 |
| Mean | 23.815 | 58.49 | 22.34 | 20.8 | 18.0 | 141.61 | 167.73 | 45.6 | 21.00 | 27.1 | 121.3 |
| Std. Dev. | 14.182 | 25.50 | 5.64 | 8.2 | 11.7 | 58.53 | 54.02 | 3.4 | 5.10 | 23.0 | 84.5 |
| Median | 24.000 | 65.70 | 23.45 | 19.5 | 15.0 | 124.30 | 162.20 | 46.0 | 20.00 | 20.0 | 92.0 |
| MAD | 17.791 | 27.72 | 6.75 | 6.7 | 8.2 | 34.84 | 50.48 | 3.0 | 4.67 | 11.9 | 51.9 |
| GMean | 18.856 | 51.46 | 21.58 | 19.6 | 0.0 | 132.54 | 160.04 | 45.5 | 20.42 | 21.7 | 99.6 |
| CV (%) | 75.558 | 55.37 | 26.96 | 33.1 | NaN | 35.09 | 30.28 | 7.4 | 23.74 | 63.3 | 61.2 |
| Min | 2.000 | 10.00 | 11.40 | 9.0 | 0.0 | 41.00 | 77.30 | 37.0 | 10.60 | 6.0 | 20.0 |
| Max | 54.000 | 114.80 | 35.40 | 76.0 | 81.0 | 466.70 | 339.40 | 54.0 | 37.00 | 211.0 | 529.0 |

TABLE 23
Summary of mean continuous covariates from the ELEKTRA population

| | Age | Weights | BMI | AST | ALT | CrCL | eGFR | Albumin | GGT | ALP |
|---|---|---|---|---|---|---|---|---|---|---|
| Statistic | (years) | (kg) | (kg/m2) | (U/L) | (U/L) | (mL/min) | (mL/min)1.73 m²) | (g/L) | (U/L) | (U/L) |
| Mean | 9.6 | 33.46 | 17.44 | 23.8 | 15.4 | 168.47 | 172.10 | 46.1 | 28.5 | 196.2 |
| Std. Dev. | 4.1 | 15.18 | 4.06 | 9.5 | 11.0 | 74.90 | 54.23 | 2.8 | 21.4 | 85.8 |
| Median | 9.0 | 29.60 | 16.60 | 22.0 | 13.0 | 157.50 | 167.00 | 46.0 | 22.0 | 183.0 |
| GMean | 8.6 | 30.25 | 17.03 | 22.5 | 13.0 | 154.28 | 164.47 | 46.0 | 23.1 | 179.1 |
| CV (%) | 50.2 | 45.79 | 21.52 | 32.3 | 55.5 | 42.31 | 30.11 | 6.0 | 63.4 | 43.4 |
| Min | 2.0 | 10.00 | 11.40 | 11.0 | 5.0 | 41.00 | 77.30 | 40.0 | 9.0 | 60.0 |
| Max | 17.0 | 73.00 | 32.60 | 76.0 | 81.0 | 466.70 | 339.40 | 53.0 | 110.0 | 529.0 |

### Model Assessment

The goodness-of-fit (GOF) plots incorporated the following modeling variables in the base and final models to assess model fit.
Individual fit plots: DV, RED, IPRED versus time, for each individual subject Scatterplots of random effects (ETAs) versus relevant covariates
Density of random effects (ETSs)
Density of residuals
Predicted residuals (IWRES, CWRES,CIWRES,NPDE) versus time (TIME) and plasma concentration (PRED)

Predicted plasma concentration versus observed plasma concentration grouped relative to covariates (PRED vs. DV; IPRED vs. DV) During the modeling process, models were compared with the parent model using a likelihood ratio test (LRT). The 5% significance level was adjusted for multiple testing. Non-nested models were compared using Akaike and Bayesian-Schwartz information criterion. Other factors considered in model comparison were parameter uncertainty (RSE), amount of explained between subject and residual variability, and interactive properties.

The base model was derived from previously developed models and graphical data exploration. Some parameter estimates used typical values (TV). Between subject variability (BSV) for PK parameters (X) were estimated using an exponential error model. THETA (.) and ETA (.) represent NONMEM placeholders for fixed and random effects, respectively. Every subject had the same fixed effect value.

$$TVX = θ ( . ) \\ X = TVX \times EXP ( ETA ( . ) )$$

BSV of Iₘₐₓ was estimated using an additive error model:

$$TVI MAX = θ ( . ) \\ I MAX = TVI MAX + ETA ( . )$$

Residual variability was described using a combined additive proportional error model that included assay variability, intra-subject variability, errors in dose timing and sample collection, subject non-compliance, model misspecification, and other unexplained errors.

$$W = SQRT ( {( THETA ( . ) / 100 \times IPRED )}^{2} + THETA ( . ) ** 2 )$$

Structural model parameters were explored by adding and dropping compartments, and testing non-linear kinetics. The residual model effects were examined by testing residual error models and transformations of the between subject variability models.

When adding a continuous or categorical covariate to the linear model to test individual random effects, an F-test determined whether unexplained variability was significantly reduced. Models including categorical covariates, such as sex, were analyzed independently to estimate their effect.

$$IF ( SEX . EQ . level ) TVX = TVX * ( 1 + THETA ( . ) / 100 )$$

Continuous covariate effects, such as age and weight, were scaled using a reference value identified using a power function.

$$TVX = TVX * ( AGE / 18 ) ** THETA ( . )$$

A visual predictive check method was used to evaluate the accuracy of the final PK and PD models. The fixed and random effect parameters from the final model were used to simulate 1000 replicates of the observed data. The 5th, 50th (median) and 95% percentile distributions of the simulated concentration values at each sampling time was calculated. Plots of the calculated data were overlaid with plots of the observed data to inspect the concordance between the simulated model and the observed data. To access the robustness of the model, a nonparametric bootstrap resampling approach was used, wherein the data was resampled 1000 times to obtain parameter estimates. The sampled parameter estimates were then compared to the estimates obtained from fitting the final model to the full dataset. By this procedure, a bootstrap mean, standard error, and bias were calculated.

A total of 2193 observations from 173 subjects was used to model the PK parameters. The modeling analysis proceeded in three steps. The first modeling step included only the oral solution formulation. The second step added the tablet arm of the studies from examples 3 and 9. The third step added the pediatric patients in the ELEKTRA study.

In step 1, several model types were tested by optimization of the Omega matrix. The omega matrix was restricted to a diagonal structure. The best model to describe the data was a linear 2-compartment model with delayed oral first-order absorption. The PK parameters included F1 (bioavailability), absorption lag time (ALAG1), absorption rate constant (KA), elimination clearance (CL), central volume (V2), inter-compartmental clearance (Q), and peripheral volume (V3). Random effects were used for F1, KA, Q, and V3. Modeling employed combined additive and proportional residual error.

Adding the tablet formulation in step 2 affected the systemic absorption lag time (ALAG1˜FORM) and the absorption rate constant (KA˜DOSE). The delay in absorption is usually described by a delay compartment; however Cₘₐₓ values were not consistent with a delay compartment model. The inconsistency could result from lower systemic clearance with the tablet formulation in the patients (CL˜PATIENT). The model demonstrated marked non-linearity. The dose effect on PK parameters identified significant covariates of relative bioavailability (F1˜DOSE), absorption rate constant (KA˜DOSE), inter-compartmental clearance (Q˜DOSE), and peripheral (V3˜DOSE).

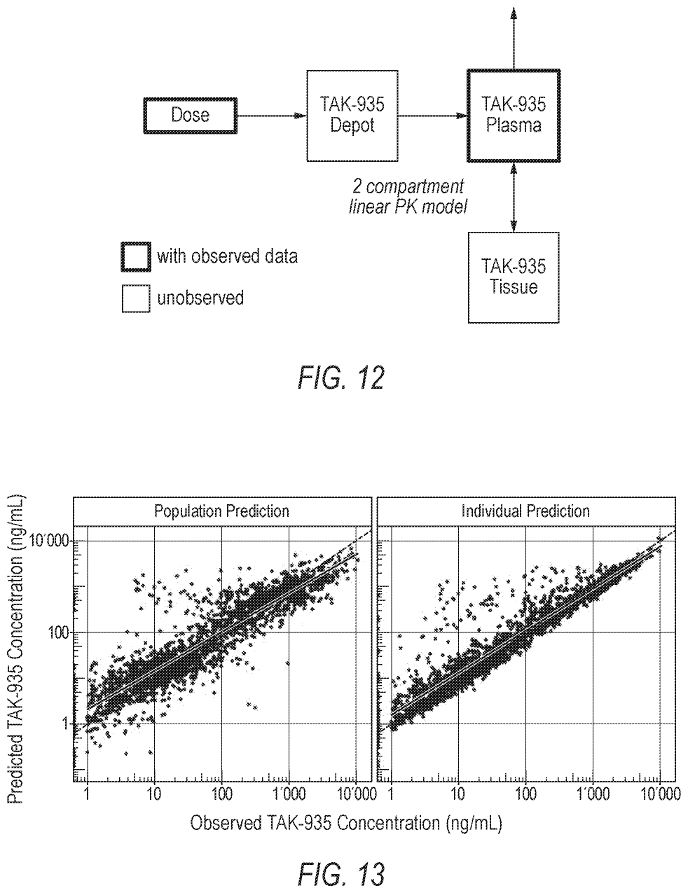

ELEKTRA dataset models showed a better fit with the tablet administration route. The growth-related changes in PK parameters were tested using the variables age, body weight, and body mass index (BMI). The growth related variables showed effects of weight on relative bioavailability (F1˜WEIGHT) and BMI on absorption rate constant in patients less than 18 years old (KA˜BMI (AGE≤18)). FIG. 12 shows a schematic of the basic drug interaction in the pharmacokinetic model. TAK-935 in the figure represents soticlestat.

Variables were screened separately by correlating the individual random effects with the variable incorporated in the model with those of the base model. An F-test was used to test the significance of the individual random effects with and without the variable. Candidates were further screened using a standard forward inclusion (α<0.05), backward deletion (α<0.01) procedure. The candidate covariates were epileptic medication with a strong inducing effect, and AST, GGT, A1-AGLP and bilirubin on F1 and KA. Other covariate candidates were Asian race and eGFR on V3. A1-AGLP produced the most significant reduction in between subject variability. However, A1-AGLP data was not collected in all studies, including the ELEKTRA study, and thus had to be imputed using the median observed value. Relative bioavailability was calculated using A1-AGLP baseline value in the following formula with A1-AGLP_MISS equal to 1 for missing; and 0 for available.

$$F 1 = F {1}_{typ} \times {( \frac{A 1 - AGLP}{20} )}^{{θ}_{1}} \times {e}^{( 1 + {θ}_{2} \times {AGP}_{MISS} ) \times η} ,$$

where θ₁ represents the power-slope of A1-AGLP on F1 and θ₂ describes the fraction of the between subject variability (n). For subjects with A1-AGLP baseline data available:

$$F 1 = F {1}_{typ} \times {( \frac{A 1 - AGLP}{20} )}^{{θ}_{1}} \times {e}^{η} ,$$

For subjects without A1-AGLP baseline data available:

$$F 1 = F {1}_{typ} \times {e}^{( 1 + {θ}_{2} ) \times η} ,$$

The remaining covariates were tested with a forward inclusion, backward deletion procedure, with forward P-value thresholds set at 0.05 and the backward threshold set at 0.01 This procedure identified the significant covariates as Asian and eGFR on V3 (V3˜eGFR, V3˜ASIAN) and “strong inducer” antiepileptic medications on the absorption rate constant (KA). Table 24 lists the shrinkage statistics for all random effect parameters. This data estimates the individual random effects (ETA) from cluster type data (e.g., different studies, repeated observations over time). The shrinkage was low to moderate with the largest variability found between subjects in Q, which is the inter-compartmental clearance. Sparse sampling at the terminal phase of the PK profile could introduce variability.

TABLE 24
Shrinkage and random effects statistics on final popPK

| Statistic | ETA (BSVKA) | ETA (BSVQ) | ETA (BSVV3) | ETA (BSVF1) | EPS (FIX) |
|---|---|---|---|---|---|
| Std. Dev. Shrinkage (%) | 20.0% | 36.6% | 16.8% | 5.2% | 7.4% |
| Num. Subjects | 173 − | 173 | 173 − | 173 | 173 |
| Mean Eta | 0.1002 | 0.024 | 0.304 | 0.014 | |
| Std. Err. Eta | 0.066 | 0.018 | 0.035 | 0.039 | |
| P (Mean Eta = 0) | 0.131 | 0.177 | 0.388 | 0.711 | |

Table 25 lists the final PK parameters and covariates tested, along with their estimated effect, relative standard error, and 95% confidence interval. In the table 25, the effects of between subject variation (BSV) on parameters is presented as standard deviation. The additive residual error was estimated to 0.001 in the final population PK model.

TABLE 25
Final population pharmacokinetic parameter estimates and their covariate effects

| Parameter | Covariate/Role | Estimate | | Fixed | Rel. Std. Err. | 95% CI |
|---|---|---|---|---|---|---|
| KA | typical value (1/h) | 7.49 | | No | 20.7% | [4.45, 10.5] |
| | non-OS formulation (%) | −45.7 | | No | 23.5% | [−66.7, −24.6] |
| | Dose exponent | −0.753 | | Yes | — | — |
| | BMI exponent | 2.52 | | No | 30.9% | [0.994, 4.05] |
| | Strong Inducer (%) | −73.7 | | No | 15.6% | [−96.2, −51.2] |
| | BSV | 1.1 | | No | 20.2% | [0.663, 1.53] |
| CL | typical value (L/h) | 4.31 | | No | 7.6% | [3.67, 4.95] |
| | Patient (%) | −24.4 | | No | 22.6% | [−35.2, −13.6] |
| V2 | typical value (L) | 2.99 | | No | 7.4% | [2.56, 3.42] |
| Q | typical value (L/h) | 1.16 | | No | 17.2% | [0.77, 1.55] |
| | Dose exponent | −0.218 | | Yes | | |
| | BSV | 0.369 | | No | 17.5% | [0.243, 0.495] |
| V3 | typical value (L) | 7.51 | | No | 18.3% | [4.82, 10.2] |
| | Dose exponent | −0.214 | | Yes | | |
| | eGFI exponent | −0.469 | | No | 44.9% | [−0.881, −0.0567] |
| | Asian (%) | −39.4 | | No | 34.4% | [−66, −12.9] |
| | BSV | 0.559 | | No | 9.1% | [0.459, 0.658] |
| ALAG1 | typical value (h) | 0.131 | | No | 9.9% | [0.106, 0.156] |
| | Non-OS formulation (%) | 25.2 | | No | 48.9% | [1.07, 49.4] |
| F1 | typical value | 0.0216 | | Yes | | |
| | Dose exponent | 0.204 | | Yes | | |
| | weight exponent | −0.516 | | No | 25.6% | [−0.775, −0.257] |
| | A1-AGLP exponent | 0.739 | | No | 37.6% | [0.195, 1.28] |
| | BSV explained by A1-AGLP (%) | 33.6 | | No | 558.0%^{b} | [−3.34, 4.01] |
| | BSV | 0.538 | | No | 14.4% | [0.387, 0.69] |
| RV | Proportional | 47.2 | | No | 3.1% | [44.3, 50.1] |
| | Additive | 0.001 | — | Yes | | |

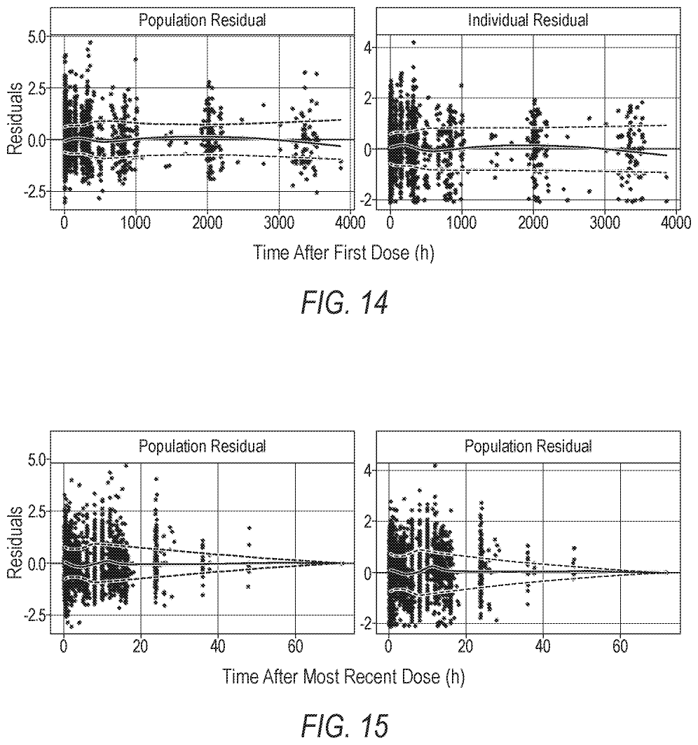

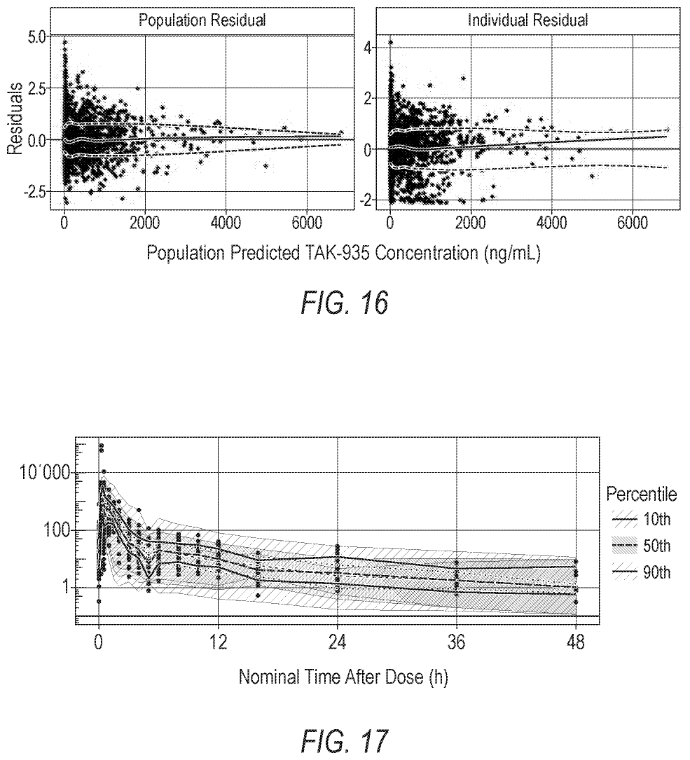

The scatterplot in FIG. 13 shows the predicted versus the observed soticlestat plasma concentration. The light gray line is the linear regression line. Both the population prediction and individual predictions align well with the observed soticlestat serum concentrations. Scatterplots in FIGS. 14-16 provide a visual comparison of the population and individual conditional weighted residuals after dose times. The residual is the difference between the predicted (regression line) and observed data. The centerline is the linear regression line of the mean, whereas the top and bottom lines are the +/−regression line of the residuals. The residual scatterplots are of time after first dose (FIG. 14), time after most recent dose (FIG. 15), and of the predicted population soticlestat concentration (FIG. 16). The y-axis shows residual values and the x-axis shows time after dose. The random pattern of residual values around the 0 residual line indicates that there was no trend over time or between population and individual data.

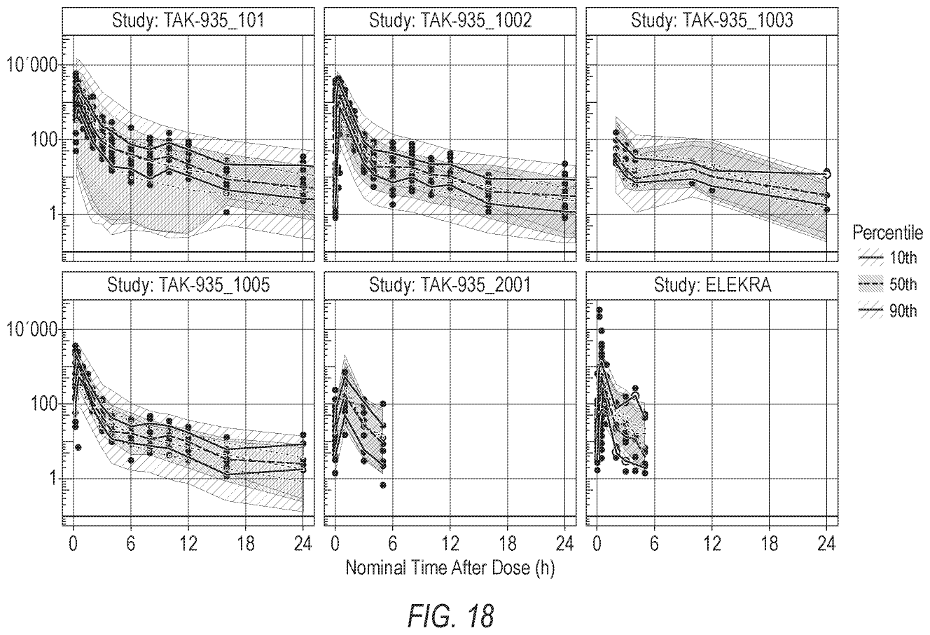

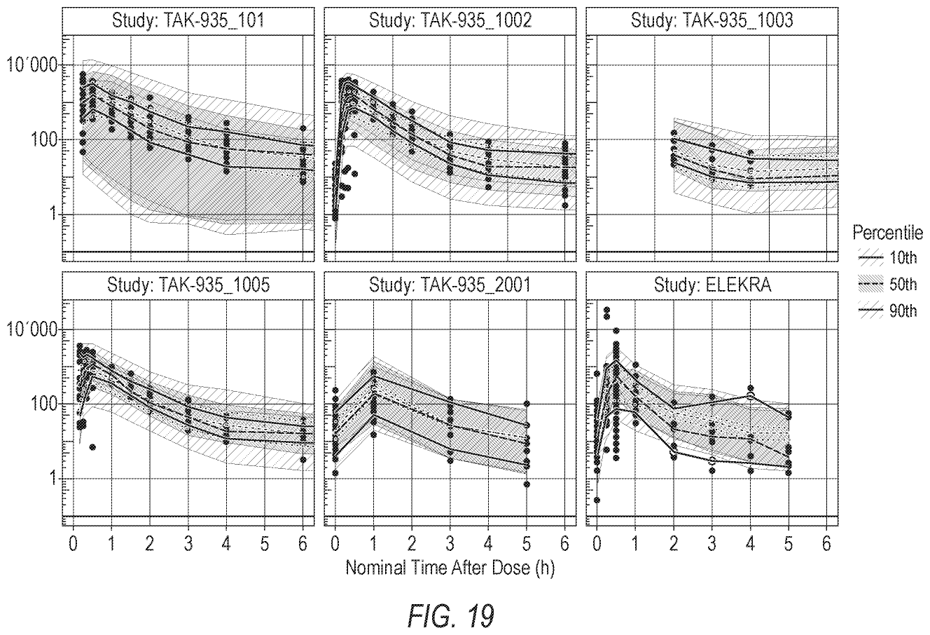

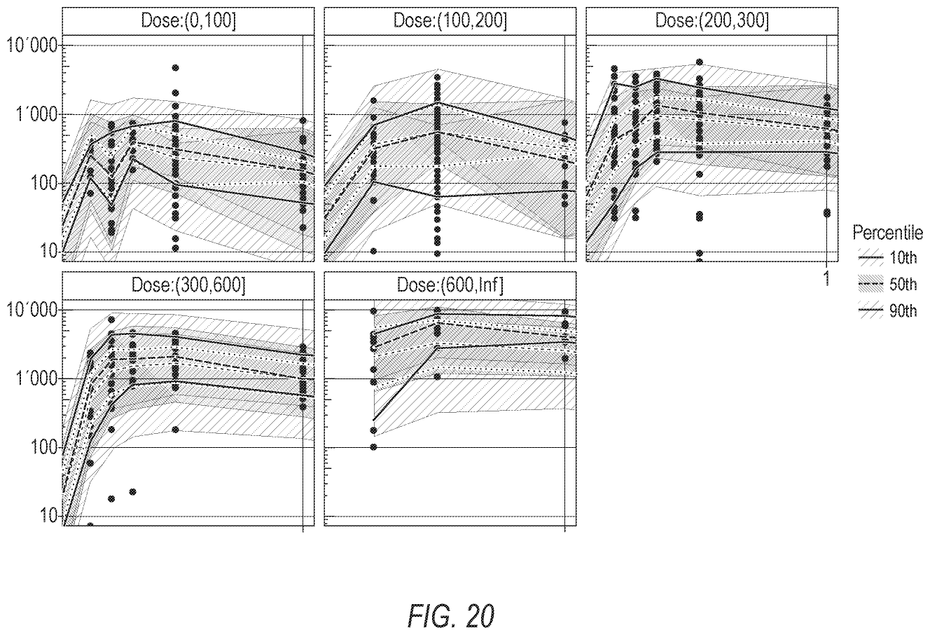

Simulation plots in FIGS. 17-20 compared the predicted-corrected (residual) PK concentration to each observed PK concentration after time of dose. These plots provided a visual predictive check (VPC) to illustrate prediction corrected simulations. The black dots represent the prediction-corrected observation. The shading represents the 95% confidence interval derived from the simulations. The dotted lines represent median simulations and the solid lines represent median observations. FIG. 17 shows the VPC from all PK concentration data up to 48 hours. FIGS. 18 and 19 compares data predicted-corrected observation from different study groups up to 24 hours and 6 hours, respectively. FIG. 20 compares the predicted-corrected observation at different dose concentrations up to 1-hour post dose and illustrates the predictive value of Cₘₐₓ. Most of the data points fall within the shaded area indicating the 95% confidence interval. A bootstrap procedure involving resampling the dataset 1000 times to create 1000 simulated samples further assessed the precision of PK parameters.

The final PK model included the following covariates and affected parameters:
Baseline A1-AGLP on relative bioavailability (F1)
Body weight on relative bioavailability (F1)
Dose on absorption rate (KA), relative bioavailability (F1), inter-compartmental clearance (Q), and peripheral volume (V3)
Patient status (healthy volunteer/patient) on systemic clearance (CL)
Body mass index on absorption rate (in subjects younger than 18 years)
Co-medication with anti-epileptic drugs (AED) that are strong PK inducers on absorption rate
eGFR on peripheral volume (V3)
Asian (yes/no) on peripheral volume (V3)

Covariates screened and found to have non-significant effects included age, sex creatinine clearance, epileptic syndrome, ethnicity, baseline albumin, several liver markers (AST, ALT, GGT, ALP, total bilirubin) and other groups of anti-epileptic drugs. The PK model could simulate a typical steady-state 24-hour PK profile for a 10-year old reference ELEKTRA patient with a body weight of 30 kg, an A1-AGLP concentration of 20 mg/cl, treated BID with 100 mg soticlestat.

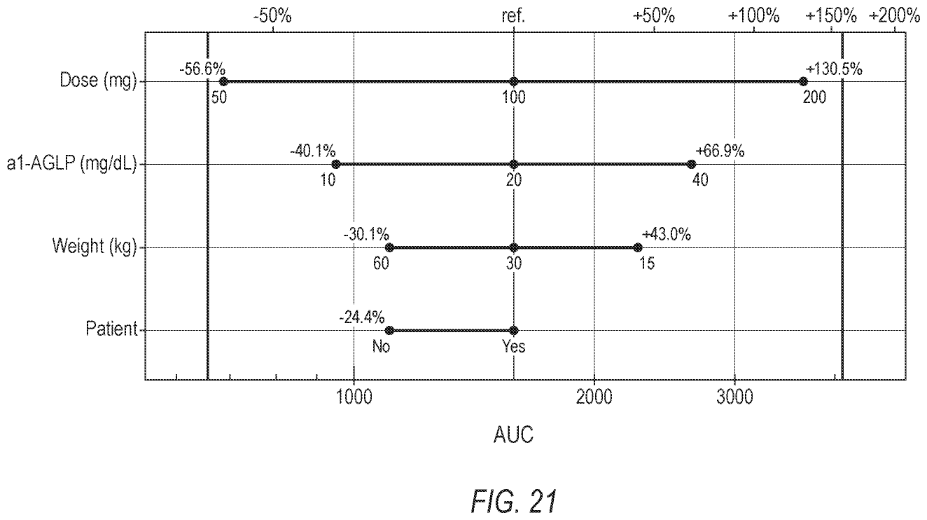

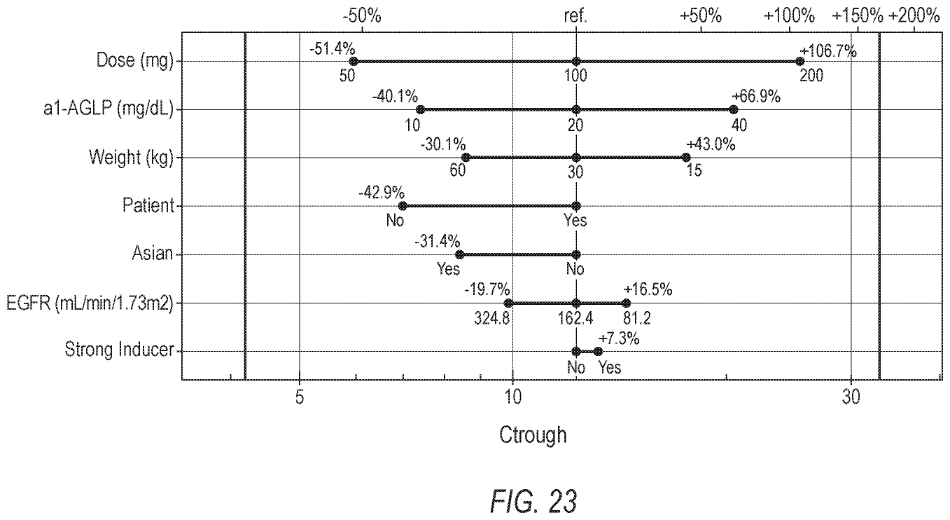

FIGS. 21-23 are tornado plots representing the change in AUC, Cₘₐₓ, and Ctrough levels after varying significant covariates one at a time at half or twice the reference value. The dots represent the simulation and the numbers below the dot represent the simulated covariate value, while the values above the dots show the percent change from the reference level. The vertical line in the center represents the reference value, while the vertical lines bracketing the covariate range depict the 80% probability interval for a reference subject. In FIG. 21, the 130% change in AUC with only twice the dosage illustrates the non-linearity dose effect on PK parameters.

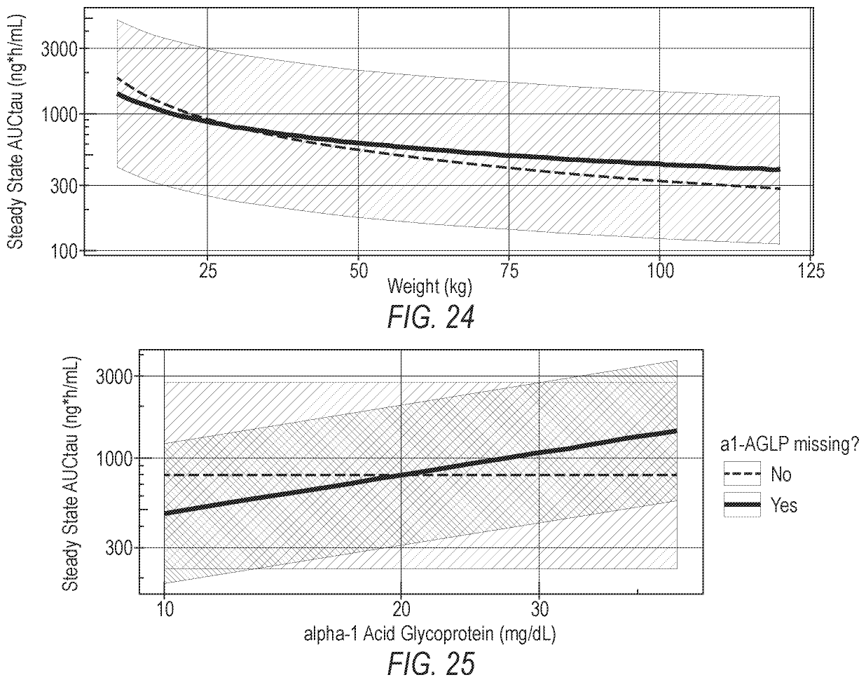

The line graph in FIG. 24 shows the effect of body weight on steady state AUC_{∞} The dashed line represents the expected allometric scaling with a weight exponent of 0.75, however the solid line shows that the actual estimated weight exponent was 0.516. FIG. 25 shows the effect of A1-AGLP concentration on steady state AUC_{∞} In the final PK model, A1-AGLP had the strongest covariate effect on soticlestat relative bioavailability.

### Example 13. Pharmacokinetic/Pharmacodynamic Modeling

The PK/PD analysis dataset consisted of 2165 24HC observations from 3 phase I trials. Four subjects from the ELEKTRA study with large IC50 estimates were excluded from the dataset. Additionally, 42 individuals with large conditional weighted residuals were excluded from the dataset. The final dataset contained 2107 24HC observations from 248 subjects. Table 26 summarizes the modeling dataset with reference to example numbers, respective clinical trial ID, subject numbers, regimen, and number of observations and doses. Tables 27 and 28 summarize the categorical covariates used in PK/OE/24HC analysis from the entire dataset and the ELEKTRA study subset, respectively.

TABLE 26
PK/EO/24HC modeling dataset details

| | | | Number Subjects | | Number | Number |
|---|---|---|---|---|---|---|
| Examples | Clinical Trial ID | Dose Number | Active | Placebo | Obs. | Doses |
| 2 | NCT02201056 | single dose | 36 | 12 | 622 | 36 |
| 6 | NCT02539134 | multiple dose | 30 | 10 | 784 | 432 |
| n/a | NCT02497235 | single dose | 11 | 0 | 88 | 11 |
| 9 | NCT03166215 | multiple dose | 17 | 0 | 165 | 2529 |
| 14 | NCT03650452 | multiple dose | 70 | 67 | 506 | 5062 |

TABLE 27
Categorical covariate data for the entire
population used for the PK/OE/24HC analysis

| Variable | Level | Count | Percent |
|---|---|---|---|
| Disease | 2001: EE | 17 | 7 |
| | 2002: Dravet S. | 51 | 20 |
| | 2002: Lennox-Gastaut S. | 86 | 34 |
| | Healthy Volunteer | 99 | 39 |
| Ethnicity | Hispanic or Latino | 50 | 20 |
| | Non-Hispanic or Latino | 203 | 80 |
| Freq. | BID | 145 | 57 |
| | QD | 108 | 43 |
| Race | American Indian or Alaska | 1 | 0 |
| | Asian | 39 | 15 |
| | Black or African American | 25 | 10 |
| | Multiracial | 1 | 0 |
| | Not Reported | 1 | 0 |
| | White | 186 | 74 |
| Sex | F | 84 | 33 |
| | M | 169 | 67 |

Tables 29 and 30 summarize the mean continuous variables used in the PK/OE/24HC covariate analysis from the entire PK/OE/24HC population studied and the ELEKTRA subset, respectively. In addition to the shared variables, the PK/PD dataset contained the individual final estimates of the population PK model parameters (IND_ALAG1, IND_KA, IND_CL, IND_V2, IND_Q, IND_V3, and IND_F1). Subjects without individual PK parameter estimates (e.g. subjects from the placebo arms) were assigned to the respective geometric mean of the estimated individual parameter values.

TABLE 28
Categorical covariate data for the ELEKTRA population
subset of the PK/OE/24HC analysis

| Variable | Level | Count | Percent |
|---|---|---|---|
| Chinese | No | 100 | 73 |
| | Yes | 37 | 27 |
| Disease | 2002: Dravet S. | 51 | 37 |
| | 2002: Lennox-Gastaut S. | 86 | 63 |
| Ethnicity | Hispanic or Latino | 21 | 15 |
| | Non-Hispanic or Latino | 116 | 85 |
| Race | American Indian or Alaska | 1 | 1 |
| | Native | | |
| | Asian | 39 | 28 |
| | Black or African American | 1 | 1 |
| | White | 96 | 70 |
| Sex | F | 49 | 36 |
| | M | 88 | 64 |

### Model Assessment

A turnover model was used to describe changes in 24HC concentration over time. Plasma 24HC was assumed to be at a steady state baseline level at the start of soticlestat dosing, with the baseline (BL) concentration maintained by a constant zero order synthesis (KIN) and first order elimination rate (KOUT), with KIN derived from BL×KOUT. Soticlestat exposure is assumed to occur mostly in the brain where it inhibits 24HC synthesis rate. Soticlestat plasma concentration (CP) was measured as an indirect exposure effect (EFF) for brain. The following formula estimates change in 24HC:

$$\frac{dHC 24}{dt} = KIN \times EFF - KOUT \times HC 24$$

The indirect exposure effect (EFF) was estimated by an Iₘₐₓ model:

$$EFF = 1 - {I}_{0} - \frac{I MAX \times {CE}^{IGAM}}{{IC}_{50}^{IGAM} + {CE}^{IGAM}}$$

Where IGAM is gamma, the shape of the concentration-probability of effect curve; IC50 is the concentration of soticlestat required to inhibit enzyme activity by 50%; and Iₘₐₓ is the soticlestat concentration required for maximum enzyme inhibition. The effect-site concentration was implemented as a delayed equilibrium with the plasma/brain transit rate parameter (KPLBR):

$$\frac{dCE}{dt} = KPLBR \times ( CP - CE )$$

TABLE 29
Summary of continuous covariate data from the entire population subset for PK/OE/24HC modeling

| | | | | | | | eGFR | | | | |
|---|---|---|---|---|---|---|---|---|---|---|---|
| | Age | Weight | BMI | AST | ALT | CrCL | (mL/min/1.73 m2 | Albumin | A1-AGLP | GGT | ALP |
| Statistic | (years) | (kg) | (kg/m2) | (U/L) | (U/L) | (mL/min) | 222 mm) | (g/L) | (umol/L) | (U/L) | (U/L) |
| N | 253 | 253 | 253 | 253 | 253 | 253 | 253 | 253 | 105 (+148 | 253 | 253 |
| | | | | | | | | | NA) | | |
| Mean | 20.6 | 52.41 | 21.16 | 21.8 | 17.7 | 146.46 | 171.91 | 45.7 | 21.01 | 28.8 | 141.4 |
| Std. Dev. | 14.0 | 25.95 | 5.67 | 9.3 | 12.6 | 58.93 | 55.15 | 3.3 | 5.05 | 27.6 | 91.7 |
| Median | 16.0 | 54.90 | 21.40 | 20.0 | 14.0 | 129.90 | 167.00 | 46.0 | 20.40 | 19.0 | 107.0 |
| MAD | 13.3 | 35.14 | 7.86 | 7.4 | 7.4 | 43.00 | 51.74 | 3.0 | 5.34 | 11.9 | 74.1 |
| GMean | 15.8 | 45.08 | 20.38 | 20.4 | 0.0 | 136.89 | 163.92 | 45.6 | 20.43 | 22.1 | 116.1 |
| SDlog | 78.1 | 58.48 | 27.90 | 36.0 | NaN | 35.87 | 30.62 | 7.2 | 23.85 | 67.9 | 62.9 |
| (%) | | | | | | | | | | | |
| Min | 2.0 | 10.00 | 9.00 | 9.0 | 0.0 | 41.00 | 69.9 | 36.0 | 10.60 | 6.0 | 30.0 |
| Max | 54.0 | 114.80 | 35.40 | 78.0 | 95.0 | 466.70 | 339.40 | 54.0 | 37.00 | 211.0 | 529.0 |

TABLE 30
Summary of continuous covariate data from the ELEKTRA population subset for PK/OE/24HC modeling

| | Age | Weight | BMI | AST | ALT | CrCL | eGFR | Albumin | GGT | ALP |
|---|---|---|---|---|---|---|---|---|---|---|
| Statistic | (years) | (kg) | (kg/m2) | (U/L) | (U/L) | (mL/min) | (mL/min/1.73 m2) | (g/L) | (U/L) | (U/L) |
| N | 137 | 137 | 137 | 137 | 137 | 137 | 137 | 137 | 137 | 137 |
| Mean | 9.6 | 33.01 | 17.45 | 24.4 | 15.2 | 166.99 | 177.21 | 45.8 | 32.3 | 201.7 |
| Std. Dev. | 4.0 | 15.72 | 4.20 | 10.6 | 11.2 | 67.79 | 55.58 | 3.1 | 30.4 | 85.2 |
| Median | 9.0 | 29.00 | 16.20 | 22.0 | 13.0 | 155.60 | 168.90 | 46.0 | 22.0 | 189.0 |
| GMean | 8.6 | 29.68 | 16.99 | 22.7 | 12.8 | 154.72 | 169.32 | 45.7 | 24.3 | 183.4 |
| CV (%) | 50.6 | 46.22 | 22.69 | 37.1 | 56.9 | 39.35 | 30.07 | 6.9 | 71.2 | 45.9 |
| Min | 2.0 | 10.00 | 9.00 | 9.0 | 5.0 | 41.00 | 77.30 | 36.0 | 8.0 | 30.0 |
| Max | 17.0 | 81.50 | 32.60 | 78.0 | 81.0 | 466.70 | 339.40 | 53.0 | 209.0 | 529.0 |

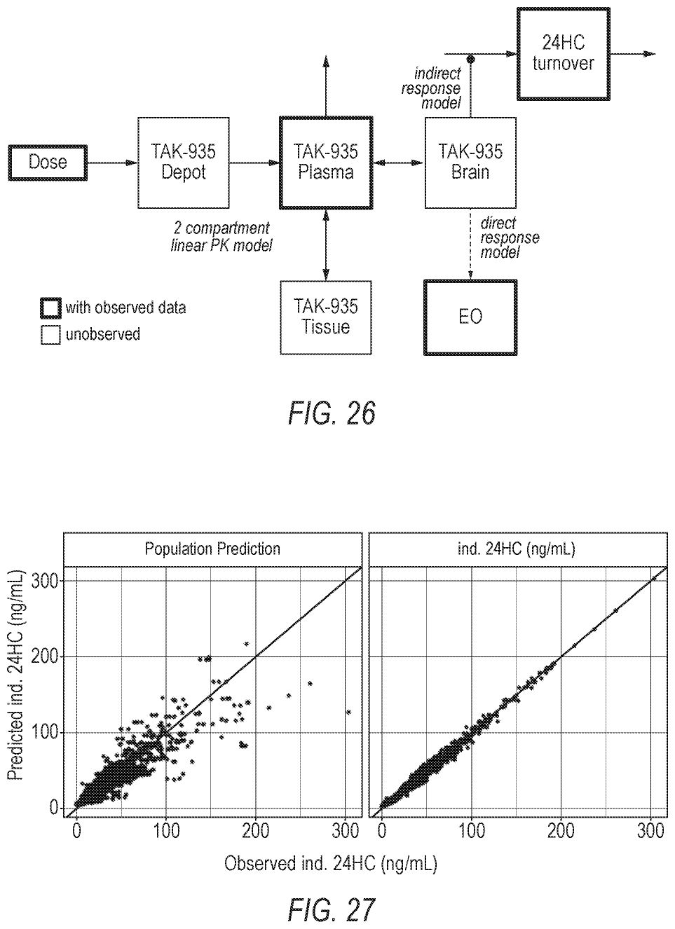

The scaling parameter KPLBR was previously estimated in a competitive enzyme occupancy model derived from PET brain imaging data (NCT02497235, Table 19). Parameters derived from this base model were fixed at their previous final PK population estimates derived from example 12. However, no random effects could be estimated without enzyme occupancy data. FIG. 26 depicts the basic model structure in relation to observed and unobserved data. There was a strong growth-related increase in baseline 24HC levels, which was only observed in young subjects. The addition of the effect site concentration estimate (CE) with regards to KPLBR did not result in significant model improvement. Thus, the model does not distinguish between plasma and brain 24HC concentration.

The PK/PD model parameters were:
Maximum inhibition of 24HC in %: Iₘₐₓ
(effect-site) concentration to reach 50% of the maximum inhibition in ng/ml: IC50
Gamma shape parameter (without units): IGAM
Plasma/brain scaling parameter in 1/h, fixed at 0.254: KPLBR

Random effects from between subject variability were estimated from baseline 24HC and soticlestat IC50.

The following variables were screened by forward inclusion, backward exclusion F-tests:
Growth/maturity: Weight, BMI, and age
Protein binding: Albumin (ALB), A1-AGLP
Liver function: AST, ALT, GGT, and ALP
Kidney function: Creatinine clearance (CRCL), eGFR
Categorical variables: Asian, sex. Ethnicity, and disease type

Weight, A1-AGLP, and GGT met the significance threshold on reducing unexplained variability by forward inclusion with P-value thresholds of 0.05 and backward exclusion with P-value of 0.01. However, the effect size of GGT was so small it is unlikely to be clinically important. Thus, the final model added two covariate relationships to the base model.
A1-AGLP on BL (backward exclusion p-value 0.000798)
Weight on BL (backward exclusion p-value 0.000033)

Table 31 lists the final PK/OE/24HC post-hoc Bayesian parameter estimates. Similar to the PK parameter estimates, a bootstrap procedure was used to confirmed the reliability of the PK/OE/24HC model parameters.

TABLE 31
Parameter estimates of the final PK/OE/24HC model

| Parameter | Role | Estimate | Fixed | Rel. Std. Err | 95% CI |
|---|---|---|---|---|---|
| Enzyme | KPLBR plasma-brain | 0.254 | Yes | | |
| Occupancy | rate | | | | |
| | EMAX maximum effect | 100 | Yes | | |
| | EC50 sensitivity | 5.86 | Yes | | |
| | EGAM shape | 0.769 | Yes | | |
| 24HC | Typical value (ng/mL) | 50.5 | No | 6.4% | [44.2, 56.9] |
| Baseline | age cut-off (y) | 21.2 | No | 0.0% | [21.2, 21.2] |
| (BL) | age exponent | −0.435 | No | 0.7% | [−0.441, −0.428] |
| | A1-AGLP exponent | 0.371 | No | 153.0% | [−0.742, 1.48] |
| | weight exponent | −0.253 | No | 0.1% | [−0.254, −0.253] |
| | BSV^{a} | 0.274 | No | 81.3% | [−0.163, 0.711] |
| KOUT | typical value (1/h) | 0.0215 | No | 0.1% | [0.0215, 0.0216] |
| Iₘₐₓ | typical value (%) | 93.5 | No | 0.0% | [93.5, 93.6] |
| IC50 | typical value (ng/mL) | 10.1 | No | 5.3% | [9.08, 11.2] |
| | BSV^{a} | 0.666 | No | 1.3% | [0.65, 0.683] |
| IGAM | typical value | 0.827 | No | 50.2% | [0.0129, 1.64] |
| RV | additive | 3.49 | No | 5.1% | [3.15, 3.84] |

The 24HC synthesis rate (KIN) was estimated at 1.09/h, which is comparable to a synthesis half-life of 0.6 hours. The 24HC elimination rate (KOUT) was estimated at 0.02 ng/mL/h, which is comparable to an elimination half-life of 1.3 days.

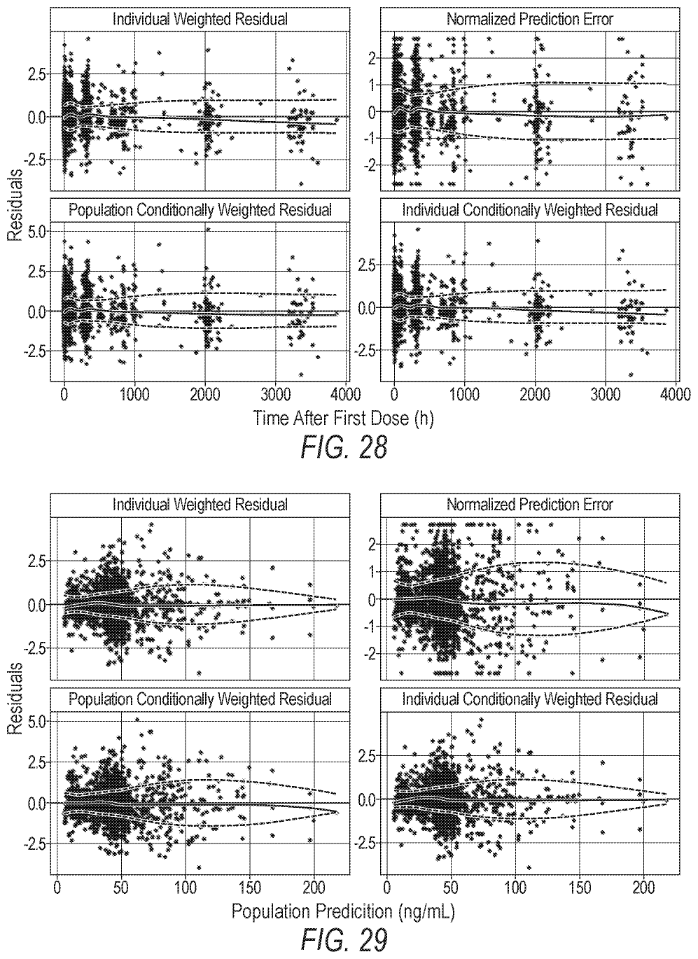

Goodness of fit plots for the final PK/EO/24HC model predictions aligned well the population and individual observed data. The scatterplot in FIG. 27 show predicted versus observed plasma 24HC concentrations. The scatterplot in FIG. 28 shows 24HC individual and population conditionally weighted residual values and individual weighted and normalized prediction residual error plotted against time after first dose. There was no trend over time apparent in the residual scatterplot. The scatterplot in FIG. 29 shows 24HC residuals versus the predicted individual and population concentrations.

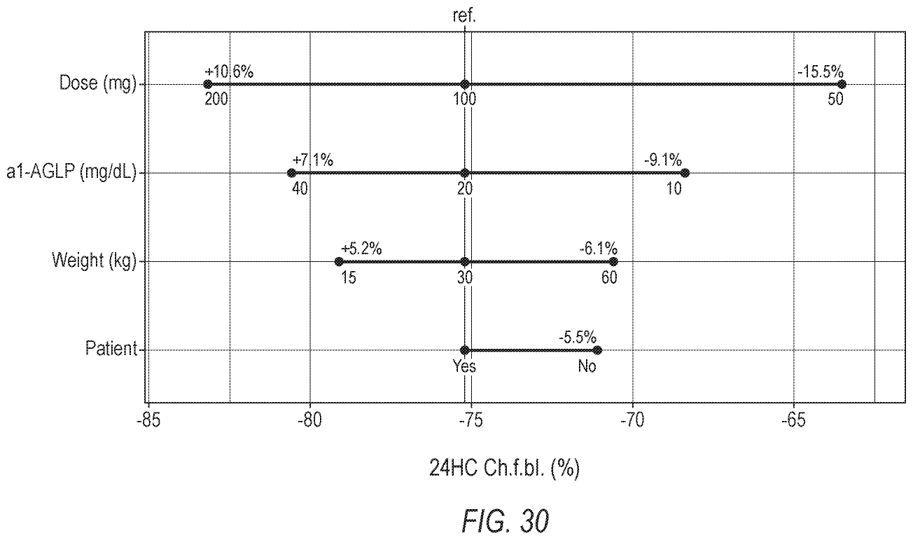

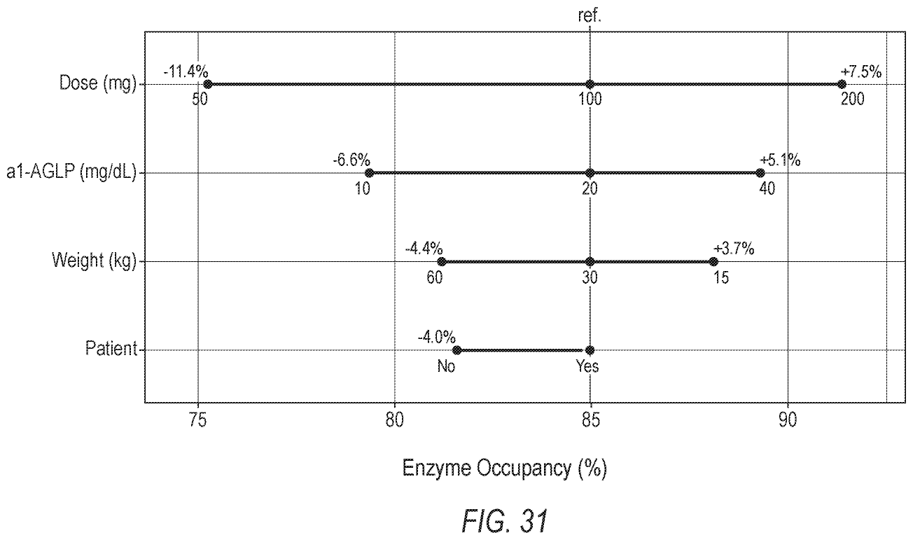

The covariate screening identified age as a covariate in subjects younger than 21 years and baseline A1-AGLP on baseline 24HC concentration. The model parameters can simulate the typical steady-state 24-hour 24HC profiles for a reference 10-year old ELEKTRA patient, weighing 30 kg, with an A1-AGLP concentration of 20 mg/dL, and treated with 100 mg soticlestat BID. The tornado plots in FIGS. 30 and 31 show the change in 24HC from baseline and enzyme occupancy after varying significant covariates one at a time at half or twice the reference value. The dots represent the simulation and the numbers below the dot represent the simulated covariate value, while the values above the dots show the percent change from the reference level. The vertical line labeled “ref” depicts the reference level. The plots only show covariates with a greater than 5% change from the reference point. Table 32 shows the final PK parameters obtained from the ELEKTRA dataset. The dosages administered to the ELEKTRA patients ranged between 60 mg and 300 mg BID. Table 33 summarize the derived statistics for PK/PD parameters for the ELEKTRA population. These values were derived from the observed individual patient data (Cₘₐₓ, Tₘₐₓ, and 24HC) and the population PK and PK/PD modeling parameter estimates. The CV % refers to the coefficient of variation assuming a log-normal distribution, derived as 100%×\sqrt{e²}−-1, where σ denotes the standard deviation of the log-transformed values.

TABLE 32
Summary of final soticlestat primary PK parameters from all subjects in the ELEKTRA study

| | Dose | ALAG1 | KA | CL/F | V2/F | Q/F | V3/F |
|---|---|---|---|---|---|---|---|
| Statistic | (mg) | (h) | (1/h) | (L/h) | (L) | (L/h) | (L) |
| Mean | 131 | 0.16 | 4.13 | 138 | 126 | 62.6 | 342 |
| Std. Dev. | 62.4 | 0 | 3.61 | 93.2 | 85.4 | 45.2 | 247 |
| G. Mean | 117 | 0.16 | 2.57 | 112 | 103 | 50.7 | 253 |
| CV % | 51.8 | 0 | 175.2 | 74.8 | 74.8 | 73.9 | 103.9 |
| Median | 120 | 0.16 | 3.045 | 114.74 | 105.17 | 50.97 | 286.525 |
| Range | 40-300 | 0.16-0.16 | 0.02-16.68 | 13.03-691.15 | 11.94-633.51 | 8.17-347.85 | 24.13-1235.17 |

TABLE 33
Summary statistics of soticlestat PK/PD
parameters from ELEKTRA subjects

| | BL | KOUT | Iₘₐₓ | IC50 | |
|---|---|---|---|---|---|
| Statistic | (ng/mL) | (1/h) | (%) | (ng/mL) | IGAM |
| Mean | 96.4 | 0.022 | 93.5 | 12.8 | 0.83 |
| Std. Dev. | 47.9 | 0 | 0 | 11.1 | 0 |
| CV % | 49.7 | 0 | 0 | 86.9 | 0 |
| Median | 84.775 | 0.022 | 93.5 | 10.06 | 0.83 |
| Range | 31.63-303.45 | 0.022-0.022 | 93.5-93.5 | 2.17-76 | 0.83-0.83 |

Table 34 list the PK exposure parameters for the ELEKTRA study, including the mean change from baseline 24HC over 24 hours at steady-state (Avg. change from baseline, (CFBL) 24HC) and the maximum reduction in baseline during steady-state (Max. CFBL 24HC). The term AUC₍₀₋ₜₐᵤ₎ stands for AUC to the end of the dosing period. The covariate effects were analyzed using exposure parameters AUC, Cₘₐₓ, and Ctrough.

The covariates affected the PK parameters absorption rate constant (Ka), clearance (CL), bioavailability (F), and peripheral volume (V3). The covariates with the most significant effects were A1-AGLP, body weight, and patients. The parameters CL/F, V2/F, Q/F, and V3/F were derived from the individual parameter and individual relative bioavailability estimates. The typical value for CL/F (clearance/bioavailability) was 150 L/h and 138.43 L for V2/F (central volume distribution).

A turnover model adequately described the relationship between the PK and 24HC data, and was confirmed using goodness of fit criteria. The magnitude of covariate effects on 24HC levels was explored by performing simulations of the percent change from baseline. The procedure identified the significant PK/PD covariates as A1-AGLP, body weight, and patients.

The model utilized typical values of 50.5 ng/mL for 24HC, a maximum inhibition of synthesis (Iₘₐₓ) of 93%, and an IC50 for 24HC of 10.1 ng/mL. The PK and PK/PD parameter estimates and covariate effect size are important estimates for predicting the optimal soticlestat dose for individuals and within a target population.

TABLE 34
Summary of statistics of soticlestat PK and PK/PD parameters from ELEKTRA patients

| | | | AUC24 | AUCtau | Cₘₐₓ | Ctrough | Avg. CFBL | Max. CFBL |
|---|---|---|---|---|---|---|---|---|
| Eq. Dose | Statistic | Dose (mg) | (ng · h/mL) | (ng · h/mL) | (ng/mL) | (ng/mL) | 24HC (%) | 24HC (%) |
| 100 mg | Mean | 72.9 | 1420 | 710 | 392 | 13.5 | −69.4 | −69.6 |
| (N = 70) | SD | 21.8 | 1080 | 542 | 275 | 25.1 | 9.85 | 9.78 |
| | G. Mean | 70.1 | 1130 | 564 | 305 | 7.86 | — | — |
| | CV % | 28 | 75.3 | 75.3 | 88.6 | 107.6 | — | — |
| | Median | 60 | 1060 | 528 | 309 | 7.28 | −70.8 | −71 |
| | Range | 40-180 | 232-5000 | 116-2500 | 22.1-1210 | 1.24-167 | −86.9-−42.2 | −87-−42.6 |
| 200 mg | Mean | 138 | 3130 | 1560 | 742 | 29.8 | −78.6 | −78.7 |
| (N = 68) | SD | 35.7 | 2400 | 1200 | 511 | 63.1 | 7.24 | 7.16 |
| | G. Mean | 134 | 2490 | 1240 | 588 | 15.7 | — | — |
| | CV % | 24.8 | 74.7 | 74.7 | 82.3 | 114.8 | — | — |
| | Median | 120 | 2420 | 1210 | 574 | 13.6 | −80.7 | −80.8 |
| | Range | 80-280 | 454-11500 | 227-5760 | 104-2140 | 2.24-404 | −89.7-−54.7 | −89.8-−55 |
| 300 mg | Mean | 196 | 4540 | 2270 | 1010 | 35.6 | −82 | −82.1 |
| (N = 56) | SD | 53.7 | 3600 | 1800 | 708 | 76.6 | 5.9 | 5.83 |
| | G. Mean | 189 | 3600 | 1800 | 789 | 21 | — | — |
| | CV % | 28.5 | 75.5 | 75.5 | 85.8 | 98.3 | — | — |
| | Median | 180 | 3460 | 1730 | 806 | 20.5 | −84 | −84.1 |
| | Range | 110-300 | 783-16900 | 391-8450 | 145-3040 | 3.65-567 | −90.9-−64.1 | −90.9-−64.4 |

Table 34A shows summary statistics from the ELEKTRA study (TAK-935-2002). Table 34A:

Summary Statistics of Dose-Dependent Parameters

| Equiv. Dose | Statistic | Avg. Ch. f. BL. 24HC (%) | Max. Red. f. BL. 24HC (%) |
|---|---|---|---|
| 100 mg BID | Mean | −67.38 | −67.65 |
| N = 70 | SD | 12.50 | 12.45 |
| | CV % | −18.6 | −18.4 |
| | Median | −70.08 | −70.39 |
| | Range | −86.70-−10.05 | −86.77-−10.19 |
| 200 mg BID | Mean | −76.77 | −76.97 |
| N = 68 | SD | 10.31 | 10.25 |
| | CV % | −13.4 | −13.3 |
| | Median | −80.21 | −80.37 |
| | Range | −89.03-−18.58 | −89.07-−18.80 |
| 300 mg BID | Mean | −79.95 | −80.11 |
| N = 56 | SD | 9.69 | 9.64 |
| | CV % | −12.1 | −12.0 |
| | Median | −82.50 | −82.65 |
| | Range | −89.98-−23.43 | −90.00-−23.69 |

Table 34B shows summary statistics for PK parameters from study TAK-935-2001. Table 34B:

Study TAK-935-2001

| | | Cmax, ss | Ctrough, ss | AUCss, 24 h | AUCss, tau |
|---|---|---|---|---|---|
| Dose | Statistic | (ng/mL) | (ng/mL) | (ng*h/mL) | (ng*h/mL) |
| 100 mg BID | N | 18 | 18 | 18 | 18 |
| | Mean | 617.11 | 12.20 | 1590.53 | 795.27 |
| | SD | 480.65 | 12.62 | 1219.15 | 609.37 |
| | CV % | 77.9 | 103.5 | 76.7 | 76.7 |
| | G. Mean | 452.45 | 7.11 | 1194.05 | 597.03 |
| | G. CV % | 107.7 | 178.0 | 98.3 | 98.3 |
| | Median | 493.00 | 8.29 | 1245.50 | 622.75 |
| | Range | 56-1780 | 0.72-50.4 | 206-4558 | 103-2279 |
| 200 mg BID | N | 17 | 17 | 17 | 17 |
| | Mean | 1274.41 | 25.17 | 3683.20 | 1841.60 |
| | SD | 1024.99 | 26.84 | 2893.28 | 1446.64 |
| | CV % | 80.4 | 106.6 | 78.6 | 78.6 |
| | G. Mean | 909.98 | 14.10 | 2719.54 | 1359.77 |
| | G. CV % | 115.4 | 191.9 | 102.3 | 102.3 |
| | Median | 978.00 | 16.60 | 2801.00 | 1400.50 |
| | Range | 105-3680 | 1.29-104 | 474.5-10500 | 237.25-5250 |
| 300 mg BID | N | 16 | 16 | 16 | 16 |
| | Mean | 1954.12 | 39.11 | 6118.38 | 3059.19 |
| | SD | 1587.03 | 42.23 | 4841.09 | 2420.55 |
| | CV % | 81.2 | 108.0 | 79.1 | 79.1 |
| | G. Mean | 1365.95 | 21.15 | 4451.62 | 2225.81 |
| | G. CV % | 123.2 | 206.1 | 107.0 | 107.0 |
| | Median | 1480.00 | 25.20 | 4673.00 | 2336.50 |
| | Range | 150-5540 | 1.82-159 | 773.1-17100 | 386.55-8550 |

### 14. Percent Change in Seizure Frequency in Pediatric Patients Enrolled in the ELEKTRA Study

The ELEKTRA study is described in the specification and in examples 12 and 13. The study measured seizure frequency as the percent change from baseline in 126 DEE patients treated with soticlestat and placebo. Baseline 24HC did not differ significantly between soticlestat and placebo. Table 35 shows the decrease in seizure frequency with soticlestat treatment compared to placebo for two time intervals. The difference in seizure frequency from baseline between soticlestat and placebo is highly significant. Statistical significance was computed using a Rank Transformed Analysis of Covariance (ANCOVA) with a 2-side p-value, while adjusting for baseline seizure frequency and indication. Tables 36 and 37 show the median percent change in seizure frequency from baseline for patients with Dravet syndrome and Lennox-Gastaut syndrome, respectively. It appears that soticlestat treatment may be especially effective in treating patients with Dravet syndrome. Tables 38 and 39 show the number of Lennox-Gastaut syndrome and Dravet syndrome patients, respectively, at different levels of percent seizure reduction. The data further indicates that soticlestat may be especially effective in patients with Dravet syndrome.

TABLE 35
Median percent change in seizure frequency from baseline

| | Placebo | Soticlestat | P value |
|---|---|---|---|
| Weeks 9-20 | | | |
| N | 56 | 64 | |
| % Change | 3.11 | −27.76 | P 0.0007 |
| Range | (−78.8 to 163) | (−100 to 160.6) | |
| Weeks 0-20 | | | |
| N | 59 | 64 | |
| % Change | 0.75 | −30.05 | P 0.0024 |
| Range | (−60.1 to 437.8) | (−100-347.5) | |

TABLE 36
Median percent change in convulsive seizure frequency
with Dravet syndrome per 28 days from baseline

| Weeks 9-20 | Placebo | Soticlestat | P value |
|---|---|---|---|
| N | 21 | 24 | |
| % Change | 9.38 | −36.5 | P 0.0001 |
| Range | (−47.3 to 153.5) | (−100 to 84.1) | |

TABLE 37
Median percent change from baseline in drop seizure frequency
per 28 days in participants with Lennox-Gastaut syndrome

| Weeks 9-20 | Placebo | Soticlestat | P value |
|---|---|---|---|
| N | 35 | 40 | |
| % Change | −1.9 | −18.46 | P 0.147 |
| Range | (−78.8 to 163) | (100.0 to 160.6) | |

TABLE 38
Number of patients with % reduction in seizures
with Lennox-Gastaut syndrome considered treatment
designated as responders in maintenance period

| | Placebo | | Soticlestat | |
|---|---|---|---|---|
| N | 37 | | 40 | |
| 25% reduction | 29.7 | (15.9-47.0) | 42.5 | (27.0-59.1) |
| 50% reduction | 16.2 | (6.2-32) | 27.5 | (14.6-43.9) |
| 75% reduction | 2.7 | (0.1-14.2) | 10 | (2.8-23.7) |
| 100% reduction | 0 | (0.0-9.5) | 5 | (0.6-16.9) |

TABLE 39
Number of patients with % reduction in seizures with Dravet
syndrome designated as responders through maintenance period

| | Placebo | | Soticlestat | |
|---|---|---|---|---|
| N | 22 | | 24 | |
| 25% reduction | 13.6 | (2.9-34.9) | 66.7 | 44.7-84.4) |
| 50% reduction | 0 | (0.0-15.4) | 41.7 | (22.1-63.4) |
| 75% reduction | 0 | (0.0-15.4) | 20.8 | (7.1-42.2) |
| 100% reduction | 0 | (0.0-15.4) | 8.3 | (1.0-27.0) |

### Example 15. Clinical Study

### Study Objectives

Primary Objective: To evaluate the safety and tolerability of TAK-935 in healthy Japanese subjects when administered as single oral dose or multiple oral doses with titration. Secondary Objective: To evaluate the PK of TAK-935 in healthy Japanese subjects when administered as single oral dose or multiple oral doses with titration.

Exploratory Objectives: To evaluate the PK of M-I, the main oxidative metabolite of TAK-935 in healthy Japanese subjects when administered as single oral dose or multiple oral doses with titration. To explore the effect of TAK-935 on 24HC plasma levels in healthy Japanese subjects when administered as single oral dose or multiple oral doses with titration.

### Trial Design

This study consisted of 2 parts, and was a randomized, double-blind, placebo-controlled study to assess safety, tolerability, and PK and PD of TAK-935 when administered orally in healthy Japanese subjects.

Part 1 had a SAD design, consisting of 3 cohorts containing 8 subjects each. On Day 1 of each dose level, a single dose of TAK-935 or placebo was administered, and safety, PK and PD were evaluated.

Part 2 had a MD design with titration study within the same subjects. TAK-935 or placebo was administered to the 9 subjects at the dose of 100 mg BID from Day 1 to Day 7, then 200 mg BID from Day 8 to Day 14, and finally 300 mg BID from Day 15 to Day 21, and safety, PK and PD were investigated. The planned dose levels of TAK-935 to be evaluated are outlined in Table 40a.

TABLE 40a
Outline of the Study Parts and Planned Dosing Cohorts

| Part | Cohort | Daily dose level (mg) | Dosing regimen | Randomization |
|---|---|---|---|---|
| Part 1 | 1 | 200 | Single oral dose under | Subjects were randomized to |
| | 2 | 600 | fasted condition | TAK-935 or placebo at a ratio |
| | 3 | 1200 | | of 6:2 in each cohort, double- |
| | | | | blind. No stratification variables |
| | | | | were used for randomization. |
| Part 2 | 4 | Day 1-7: 200 (100 mg BID) | Multiple oral doses up- | Subjects were randomized to |
| | | Day 8-14: 400 (200 mg BID) | titration with TAK-935 | TAK-935 or placebo at a ratio |
| | | Day 15-21: 600 (300 mg BID) | for 7 days per each dose | of 6:3, double-blind. No |
| | | | level within the same | stratification variables were |
| | | | subjects under fasted | used for randomization. |
| | | | condition | |

BID: twice daily

### Study Population: Healthy Japanese Subjects.

Planned Number of Subjects
Part 1 (SAD): Total of 24 subjects
Cohort 1:8 subjects, Cohort 2:8 subjects, Cohort 3:8 subjects
Part 2 (MD): Total of 9 subjects
Cohort 4:9 subjects (dose up-titration within the same subjects)
Key Study Procedure Overview

### <Part 1>

After the screening visit, eligible participants checked—in on Day-2. On Day 1, eligible participants were randomized 6:2 to TAK-935 or placebo in each dose group in double-blinded fashion. No stratification variables were used for randomization. On the same day, study drug was administered by oral administration of single dosing of TAK-935 to each cohort at each dose level. The subjects were discharged on Day 3. Intensive PK samplings were performed from Day 1 to Day 3.

### <Part 2>

After the screening visit, eligible participants checked—in on Day-2. On Day 1, eligible participants were randomized 6:3 to TAK-935 or placebo in double-blinded fashion. No stratification variables were used for randomization. Study drug was administered every day by oral administration of multiple dosing with up-titration of TAK-935 to 9 subjects firstly at 100 mg BID from Day 1 to Day 7, then at 200 mg BID from Day 8 to Day 14, and finally at 300 mg BID from Day 15 to Day 21, successively. The subjects were discharged on Day 24. Intensive PK samplings were performed on Day 1, Day 7, Day 14 and Day 21.

### Dose Escalation

### <Part 1>

Blinded safety and PK were evaluated at the end of treatment for each cohort and prior to dose escalation.

### <Part 2>

Part 2 (Cohort 4) was initiated after the evaluation of blinded safety and PK at 600 mg single dose in Part 1 (Cohort 2).

The dose escalation scheme is shown below in Table 40b:

TABLE 40b

| Part 1 (Single dose) | | | Part 2 (Multiple dose) |
|---|---|---|---|
| Cohort ^{a}→ | Cohort 2 ^{a}→ | Cohort 3 * | Cohort 4 ^{b} |
| TAK-935 200 mg or | TAK-935 600 mg or | TAK-935 1200 mg or | TAK-935 100 mg BID or placebo, then |
| placebo | placebo | placebo | TAK-935 200 mg BID or placebo, and |
| | | | finally TAK-935 300 mg BID or |
| | | | placebo with up-titration within the |
| | | | same subjects |

BID: twice daily; PK: pharmacokinetics.
^{a}The subsequent cohort was run after the evaluation of blinded safety and PK in the prior cohort and confirmed that the prior cohort was well tolerated.
^{b} Cohort 4 was initiated after the evaluation of blinded safety and PK in Cohort 2.

If the Cₘₐₓ and/or the AUC were expected to be much greater than the maximum exposure at maximum dose evaluated in precedent clinical studies, the dose level in the subsequent cohort was to be lower than the planned dose.

Treatments Administered

### <Part 1>

Subjects received a single dose of TAK-935 or placebo of 200 mg, 600 mg, or 1200 mg with 150 mL of water, after fasting for at least 10 hours and remained fasted until 4 hours after dosing.

In Cohort 3, in which 1200 mg of study drug was administered, subjects could ingest up to 150 ml of water as needed (ie, subjects in this cohort were not required to ingest the full 150 mL of water).

### <Part 2>

Subjects received multiple oral doses of TAK-935 or placebo escalating from 100 mg BID, 200 mg BID, and then to 300 mg BID with 150 mL of water for 7 days per each dose level. For the morning dose, subjects were kept fasted for at least 8 hours before administration and up to 2 hours after administration. The evening dose was administered at any time between 8 hours and 12 hours after the morning dose, but prior to the evening meal except for Days 1, 7, 14, and 21. On Days 1, 7, 14, and 21, the evening dose was administered 12 hours after the morning dose and prior to the evening meal.
Identity of Study Drugs
Study drug refers to TAK-935 100 mg tablet and matching placebo.
Compound: Code name: TAK-935
Dosage forms: TAK-935 100 mg tablets and matching placebo tablets were used in this study. The TAK-935 tablets and placebo tablets were yellow-red film-coated tablets for oral administration. A film coating was applied to protect against the photosensitivity exhibited by the uncoated tablets.
Manufacturing: TAK-935 100 mg tablets and matching placebo tablets were manufactured by SPERA Pharma Inc. Osaka, Japan. Table 40d shows identity of study drugs in this study.

TABLE 40c
Schedule of Study Procedures
Part 1

| | Screening | | | | | | | |
|---|---|---|---|---|---|---|---|---|
| | Days | Check-in | Baseline | Treatment | | Study Exit | Follow-up | Early |
| | −28 to −3 | Day −2 | Day −1 | Day 1 | Day 2 | Day 3 | Day 8 | Termination |
| Informed consent | x | | | | | | | |
| Inclusion/exclusion criteria | x | x | x | x | | | | |
| Demographics and medical history | x | | | (predose) | | | | |
| Medication history | x | | | | | | | |
| Physical exam | x | | x | x | | x | x | x |
| | | | | (predose) | | | | |
| Eye exam [symbol] | | | x | | | | x | x |
| [symbol] assessment | | | x | | | x | x | x |
| (MMSE and pre-determined | | | | | | | | |
| questions) | | | | | | | | |
| Vital signs [symbol] | x | x | x | x | x | x | x | x |
| Weight, height, and BMI [symbol] | x | x | | | | x | | x |
| [symbol] | x | x | x | x | x | x | x | x |
| Concurrent medical conditions | x | x | | | | | | |
| Clinical laboratory evaluations [symbol] | x | | x | | | x | | x |
| Hormone laboratory tests [symbol] | | | x | | | x | | x |
| [symbol] | x | | | | | | | |
| HIV, [symbol] | x | | | | | | | |
| Serum FSH (if applicable) | x | | | | | | | |
| Urine drug and alcohol screen | x | | x | | | | | |
| Standard 12-lead ECG [symbol] | x | | | x | x | x | | x |
| Holter ECG [symbol] | | | x | x | x | | | |
| Confinement [symbol] | | x | x | x | x | x | | |
| PK blood collection [symbol] | | | | x | x | x | | x |
| 24HC level blood collection [symbol] | | | x | x | x | x | | |
| PGx DNA collection [symbol] | | | | x | | | | |
| Study drug dosing | | | | x | | | | |
| C-SSRS assessment | x | x | | | | x | | x |
| AE assessment [symbol] | x | x | x | x | x | x | x | x |

AE: adverse event; BMI: body mass index; C-SSRS: Columbia Suicide Severity Rating Scale; ECG: electrocardiogram; FSH: follicle stimulating hormone; MMSE: mini [symbol] state examination; PD: pharmacodynamic(s); [symbol] PK: pharmacokinetic(s); TSH: thyroid stimulating hormone; 24HC: 24S-hydroxycholesterol [symbol]
Eye exam included visual field evaluation, best corrected visual [symbol] without pupillary dilation, and a slit lamp microscopy. [symbol]
Vital signs (temperature, respiration, pulse, and blood pressure) were obtained at Screening, Check-in (Day [symbol] ), Day [symbol] (upon rising), Day [symbol] (predose, and at 1, 4, 12 hours postdose), Day 2 (at 24 hours postdose), Discharge (at 48 hours postdose) and Follow-up or Early [symbol] [symbol]
Height and BMI were only collected at Screening. [symbol]
Record all ongoing medications from Screening and throughout the study. [symbol]
Clinical laboratory tests (hematology, serum chemistry, and urinalysis) were collected at Screening, Day [symbol] (upon rising) and Discharge (at 48 hours postdose) or Early Termination under fasted conditions. [symbol]
Hormone laboratory tests ( [symbol] , growth hormone, cortisol, and TSH) were collected on Day [symbol] (upon rising), and Discharge (upon rising) or Early Termination under fasted conditions. [symbol]
Standard 12-lead ECG was recorded at Screening, Day 1 (predose, and at 1, 2, 4, 8, 12 hours postdose), Day 2 (at 24 hours postdose) and Discharge (at 48 hours postdose) or Early Termination. [symbol]
QT extraction time points were the same anticipated time points matching Day [symbol] on Day [symbol] , predose, 0.25, 0.5, 1, 1.5, 2, 4, 8, 10, 12, 16 and 24 hours postdose. [symbol]
Subjects were discharged on Day [symbol] . [symbol]
Blood samples for pharmacokinetic analyses were collected at predose and at 0.25, 0.5, 1, 1.5, 2, 4, 8, 10, 12, 16, 24 and 48 hours postdose or Early Termination. [symbol]
Blood samples for PD analysis were collected on Day [symbol] at the same anticipated time points matching Day 1, [symbol] at predose, 1, 4, 8, 12 hours postdose, Day 2 (at 24 hours postdose) and Day 3 (at 48 hours postdose). [symbol]
One blood sample was collected for PGx DNA analysis on Day [symbol] . [symbol]
Any event from signing of informed consent was captured as an AE. [symbol]
indicates data missing or illegible when filed

TABLE 40d
Part 2

| | Screening | Check- | | Dose Level 1 | | | Dose Level 2 | |
|---|---|---|---|---|---|---|---|---|
| | Days −28 | in | Baseline | Day | Day | Day | Day | Day |
| | to −3 | Day −2 | Day −1 | 1 | 2-6 | 7 | 8 | 9-13 |
| Informed consent | x | | | | | | | |
| Inclusion/exclusion criteria | x | x | x | x | | | | |
| | | | | (predose) | | | | |
| Demographics and medical | x | | | | | | | |
| history | | | | | | | | |
| Medication history | x | | | | | | | |
| Physical exam | x | | x | | | | x | |
| | | | | | | | (predose) | |
| Eye exam [symbol] | | | x | | | | | |
| Neuropsychiatric | | | x | | | x | | |
| assessmet | | | | | | | | |
| (MMSE and pre-determined | | | | | | | | |
| questions) | | | | | | | | |
| Vital signs [symbol] | x | x | x | x | x | x | x | x |
| Weight, height, and BMI [symbol] | x | x | | | | | | |
| [symbol] | x | x | x | x | x | x | x | x |
| Concurrent medical | x | x | | | | | | |
| conditions | | | | | | | | |
| Clinical laboratory | x | | x | | | | x | |
| evaluations [symbol] | | | | | | | | |
| Hormone laboratory tests [symbol] | | | x | | | | x | |
| Hepatitis panel | x | | | | | | | |
| HIV, Syphilis | x | | | | | | | |
| [symbol] | x | | | | | | | |
| [symbol] | x | | x | | | | | |
| Standard 12-lead ECG [symbol] | x | | | x | | | x | |
| Holter ECG [symbol] | | | x | x | | | | |
| Confinement [symbol] | | x | x | x | x | x | x | x |
| PK blood collection [symbol] | | | | x | | x | | |
| 24HC level blood collection [symbol] | | | x | x | x | | x | x |
| [symbol] | | | | x | | | | |
| Study drag dosing | | | | x | x | x | x | x |
| C-SSRS assessment | x | x | | | | | | |
| AE assessment [symbol] | x | x | x | x | x | x | x | x |

| | | | | | | Study | Follow- | |
|---|---|---|---|---|---|---|---|---|
| | Dose Level 2 | Dose Level 3 | | | | Exit | up | |
| | Day | Day | Day | Day | Day | Day | Day | Early |
| | 14 | 15 | 16-20 | 21 | 22-23 | 24 | 35 | Termination |
| Informed consent | | | | | | | | |
| Inclusion/exclusion criteria | | | | | | | | |
| Demographics and medical | | | | | | | | |
| history | | | | | | | | |
| Medication history | | | | | | | | |
| Physical exam | | x | | | | x | x | x |
| | | (predose) | | | | | | |
| Eye exam [symbol] | | | | | | | x | x |
| Neuropsychiatric | x | | | x | | x | x | x |
| assessmet | | | | | | | | |
| (MMSE and pre-determined | | | | | | | | |
| questions) | | | | | | | | |
| Vital signs [symbol] | x | x | x | x | x | x | x | x |
| Weight, height, and BMI [symbol] | | | | | | x | | x |
| [symbol] | x | x | x | x | x | x | x | x |
| Concurrent medical | | | | | | | | |
| conditions | | | | | | | | |
| Clinical laboratory | | x | | | x | | | x |
| evaluations [symbol] | | | | | | | | |
| Hormone laboratory tests [symbol] | | x | | | x | | | x |
| Hepatitis panel | | | | | | | | |
| HIV, Syphilis | | | | | | | | |
| [symbol] | | | | | | | | |
| [symbol] | | | | | | | | |
| Standard 12-lead ECG [symbol] | | x | | x | x | x | | x |
| Holter ECG [symbol] | x | | | x | | | | |
| Confinement [symbol] | x | x | x | x | x | x | | |
| PK blood collection [symbol] | x | | | x | | | | x |
| 24HC level blood collection [symbol] | | x | x | x | x | | | |
| [symbol] | | | | | | | | |
| Study drag dosing | x | x | x | x | | | | |
| C-SSRS assessment | | | | | | x | | x |
| AE assessment [symbol] | x | x | x | x | x | x | x | x |

AE: adverse event; BMI: body mass index; C-SSRS: Columbia Suicide Severity Rating Scale; ECG: electrocardiogram; FSH: follicle-stimulating hormone; MMSE: mini mental state examination; PD: [symbol] PK: pharmacokinetic(s); TSH: thyroid stimulating hormone; [symbol] [symbol]
Eye exam included visual field evaluation, best corrected visual [symbol] without pupillary dilation, and a slit lamp microscopy. [symbol]
Vital signs (temperature, respiration, pulse, and blood pressure) were obtained at Screening, Check-in (Day −2) [symbol] Day [symbol] (upon rising), Days [symbol] through 20 (each pre-morning dose), Day 21 (pre-morning dose, and at 1, 4, 12 hours after morning dose), Day 22 (at 24 hours after final morning dose), Day 23 (at 48 hours after final morning dose), Study Exit (at 72 hours after final morning dose), and follow-up of Early Termination. [symbol]
Height and BMI were only collected at Screening. [symbol]
Record all ongoing medications from Screening and throughout the study. [symbol]
Clinical laboratory tests (hematology, serum chemistry, and urinalysis) were collected at Screening, Day [symbol] (upon rising), Day 8, Day 15 (each pre-morning dose), and Day 22 (at 24 hours after final morning dose) or Early Termination under fasted conditions. [symbol]
Hormone laboratory tests (prolactin, growth hormone, cortisol, and TSH) were collected on Day [symbol] (upon rising), Day 8 (upon rising), Day 15 (upon rising), and Day 22 (upon rising) or Early Termination under fasted conditions. [symbol]
Standard 12-lead ECG was recorded at Screening, Day 1, Day 8, Day 15 (each pre-morning dose), Day 21 (pre-morning dose, and at 1, 2, 4, 8, 12 hours after morning dose) and Day 22 (at 24 hours after final morning dose), Day 23 (at 48 hours after final morning dose), and Study Exit (at 72 hours after final morning dose) or Early Termination. [symbol]
QT extraction time points were the same anticipated time points matching Day [symbol] on Day [symbol] , pre-morning dose and 0.25, 0.5, 1, 2, 4, 8, 10 and 12 hours after morning dose on Day 1, Day 14 and Day 21. [symbol]
Subjects were discharged on Day 24 [symbol]
Blood samples for pharmacokinetic analyses were collected at pre-morning dose and at 0.25, 0.5, 1, 2, 4, 8, 10 and 12 hours after morning dose on Day 1, Day 7, Day 14 and Day 21, or Early Termination. [symbol]
Blood samples for PD analysis were collected on Day [symbol] , Day 2, Day 4, Day 8, Day 11, Day 15, Day 18 (each pre-morning dose) and on Day [symbol] at the same anticipated time points matching Day 21, at pre-morning dose, 1, 4, 8, 12 and 24 hours after final morning dose of Day 21 to Day 22. [symbol]
One blood sample was collected for PGx DNA analysis on Day 1. [symbol]
Any event from signing of informed consent was captured as an AE. [symbol]
indicates data missing or illegible when filed

### Drug Concentration Sample Collection

Blood samples for PK analysis of TAK-935 and its metabolites (M-I) were collected into blood collection tubes (vacutainer) containing the anticoagulant K2EDTA (Table 40e and Table 40f).

TABLE 40e
Sampling of Blood Samples for PK Analysis (Part 1)

| Analyzed substances | Samples | Study date | Blood sampling time |
|---|---|---|---|
| TAK-935 and its | Plasma | Day 1-3 | Predose, 0.25, 0.5, 1, 1.5, 2, 4, 8, 10, 12, 16, 24 and 48 hours post |
| metabolite (M-I) | | | dose or Early Termination. |

PK: pharmacokinetic(s).

TABLE 40f
Sampling of Blood Samples for PK Analysis (Part 2)

| Analyzed substances | Samples | Study date | Blood sampling time |
|---|---|---|---|
| TAK-935 and its | Plasma | Day 1, 7, | Pre-morning dose and at 0.25, 0.5, 1, 2, 4, 8, 10 and 12 hours after |
| metabolite (M-I) | | 14, 21 | morning dose on Day 1, Day 7, Day 14 and Day 21, or Early |
| | | | Termination |

PK: pharmacokinetic(s).

### Pharmacokinetic Parameter Estimates

The PK parameters of TAK-935 and its metabolites (M-I) were determined from the concentration-time profiles for all evaluable subjects. Actual sampling times, rather than scheduled sampling times, were used in all computations involving sampling times. The following PK parameters were calculated from plasma concentrations of TAK-935 and its metabolites (M-I), unless otherwise specified:

| Symbol/Term | |
|---|---|
| Plasma | Definition |
| Cₘₐₓ | Maximum observed plasma concentration |
| AUCₗₐₛₜ | Area under the plasma concentration-time curve from |
| | time 0 to time of the last quantifiable concentration |
| | (Part 1 only) |
| AUC_{∞} | Area under the plasma concentration-time curve from |
| | time 0 to infinity (Part 1 only) |
| AUC₂₄ | Area under the plasma concentration-time curve from |
| | time 0 to 24 hours (Part 1 only) |
| AUC [symbol] | Area under the plasma concentration time carve during |
| | a dosing interval (Part 2 only) |
| tₘₐₓ | Time of first occurrence of Cₘₐₓ |
| t_{1/2} [symbol] | Terminal disposition phase half-life |
| CL/F | Apparent clearance after extravascular administration |
| | (for TAK-935 only) |
| V_{z}/F | Apparent volume of distribution during the terminal |
| | phase (for TAK-935 only) |
| MR | Metabolic ratio based on AUC |
| R_{ac(AUC)} | Accumulation ratio based on AUC (Part 2 only) |
| R_{ac(Cmax)} | Accumulation ratio based on Cₘₐₓ (Part 2 only) |

[symbol]

indicates data missing or illegible when filed

### Pharmacodynamic Sample Collection

Blood samples for PD analysis of plasma 24HC level were collected into blood collection tubes (vacutainer) containing the anticoagulant K2EDTA.

The actual time of sample collection was recorded on the source document and eCRF. Sampling time points were to be adjusted based on the preliminary emerging concentration data collected from prior subject(s), but the total number of samples collected per subject was not to exceed the planned number.

TABLE 40g
Sampling of Blood Samples for PD Analysis (Part 1)

| Analyzed | | Study | |
|---|---|---|---|
| substances | Samples | date | Blood sampling time |
| 24HC | Plasma | Day | Day −1 at the same anticipated time points |
| | | 1-3 | matching Day 1, ie at predose, 1, 4, 8, 12 |
| | | | hours postdose, Day 2 (at 24 hours postdose) |
| | | | and Day 3 (at 48 hours postdose) |

PD: pharmacodynamic(s); 24HC: 24S-hydroxycholesterol.

TABLE 40h
Sampling of Blood Samples for PD Analysis (Part 2)

| Analyzed | | Study | |
|---|---|---|---|
| substances | Samples | date | Blood sampling time |
| 24HC | Plasma | Day | Day 1, Day 2, Day 4, Day 8, Day 11, Day |
| | | 1-22 | 15, Day 18 (each pre-morning dose) and |
| | | | on Day −1 at the same anticipated time |
| | | | points matching Day 21, at pre-morning |
| | | | dose, 1, 4, 8, 12 and 24 hours after |
| | | | morning dose of Day 21 to Day 22 |

PD; pharmacodynamic(s); 24HC: 24S-hydroxycholesterol.

The PD assessments described below were performed at the time points stipulated in the schedule of procedures. The additional PD endpoints included the following PD parameters for plasma 24HC:

| Symbol Term | |
|---|---|
| Plasma | Definition |
| AUEC₂₄ | Area under the effect time curve from time 0 to 24 hours |
| | (Part 1 and Part 2) |
| AUEC [symbol] | Area under the effect-time curve from time 0 to last |
| | sampling time point ( [symbol] ) (Part 1 only) |
| AUEC [symbol] | Area under the effect time curve during a dosing interval |
| | (Part 2 only) |
| Eₘₐₓ | Maximum drug-induced effect (Part 1 and Part 2) |
| E₂₄ | Observed effect at 24 hours (Part 1 and Part 2) |
| E₁₂ | Observed effect at 12 hours (Part 2 only) |
| Time to Eₘₐₓ | Time to reach maximusn PD effect |
| %Δ AUEC₂₄ | Percentage change from Baseline for AUEC₂₄ (Part 1 and |
| | Part 2) |
| %Δ AUEC [symbol] | Percentage change from Baseline for AUEC [symbol] (Part 2 |
| | only) |
| %Δ Eₘₐₓ | Percentage change from Baseline for Eₘₐₓ (Part 1 and |
| | Part 2) |
| %Δ E₂₄ | Percentage change from Baseline for E₂₄ (Part 1 and Part |
| | 2) |
| %Δ E₁₂ | Percentage change from Baseline for E₁₂ (Part 2 only) |

[symbol]

indicates data missing or illegible when filed

### Endpoints

The primary endpoint of the study included: Safety endpoint: Percentage of subjects who experience at least 1 TEAE Secondary endpoints included:

### Pharmacokinetic Endpoints:

Maximum observed plasma concentration (Cₘₐₓ).
Area under the plasma concentration-time curve from time 0 to time of the last quantifiable concentration (AUClast) (Part 1 only).
AUC from time 0 to time of infinity (AUC_{∞}) (Part 1 only).
AUC from time 0 to 24 hours (AUC₂₄) (Part 1 only).
AUC during a dosing interval (AUCτ) at steady state (Part 2 only).
   Exploratory Endpoints were Assessed Through the Following Parameters:

### Safety Endpoints:

Laboratory tests.
Vital signs.
ECG parameters.

The PK endpoints included the following PK parameters of TAK-935 and its metabolite M-I:
Cₘₐₓ for M-I.
AUClast for M-I (Part 1 only).
AUC_{∞} for M-I (Part 1 only).
AUC₂₄ for M-I (Part 1 only)
AUCt for M-I (Part 2 only).
Time to Cₘₐₓ (Tₘₐₓ).
Terminal disposition phase half-life (t1/2z).
Apparent clearance after extravascular administration (CL/F) (for TAK-935 only).
Apparent volume of distribution during the terminal disposition phase after extravascular
   administration (Vz/F) (for TAK-935 only).
MR based on AUC.
Accumulation index (Part 2 only)
   Additional PK parameters were to be included if appropriate.
   The PD endpoints included the following PD parameters for 24HC:
Area under the effect-time curve from time 0 to 24 hours (AUEC₂₄) (Part 1 and Part 2).
Area under the effect-time curve from time 0 to last sampling time point (t) (AUEC_{τ}) (Part 1 only)·
Area under the effect-time curve during a dosing interval (AUEC_{τ}) (Part 2 only).
Maximum drug-induced effect (Emax) (Part 1 and Part 2).
Observed effect at 24 hours (E24) (Part 1 and Part 2).
Observed effect at 12 hours (E12) (Part 2 only).
Time to reach maximum PD effect (time to Emax) (Part 1 and Part 2).
Percent change from Baseline for AUEC₂₄ (Part 1 and Part 2).
Percent change from Baseline for AUECτ (Part 2 only).
Percent change from Baseline for Emax (Part 1 and Part 2).
Percent change from Baseline for E24 (Part 1 and Part 2).
Percent change from Baseline for E12 (Part 2 only).

Additional PD parameters were to be included if appropriate.

Plasma Concentrations and Pharmacokinetic Parameter Results of TAK-935 (Cohort 1, 2, and 3)

A summary of plasma PK parameters of TAK-935 following a single dose is presented in Table 40g. A summary of dose-normalized plasma PK parameters of TAK-935 following a single dose is presented in Table 40h.

TAK-935 was rapidly absorbed with Tₘₐₓ between 0.5000 to 0.7500 hour (median Tₘₐₓ) and mean t1/2z values ranged from 5.075 to 8.695 hours following a single dose of TAK-935 200, 600, or 1200 mg. Mean Cₘₐₓ values were 850.0, 3282, and 10150 ng/ml for 200, 600, and 1200 mg administration, respectively. Mean AUC₂₄ values were 698.3, 3857, and 11290 h*ng/ml for 200, 600, and 1200 mg administration, respectively, with similar values observed for AUClast and AUC_{∞}. Intersubject variability (percent coefficient of variation [% CV]) for TAK-935 ranged from 47.9% to 139.3% for Cₘₐₓ and from 42.8% to 90.0% for AUC₂₄, AUClast, and AUC_{∞}.

Following a single dose of TAK-935 200, 600, or 1200 mg, dose-normalized Cₘₐₓ values were increased with increasing dose, ranging from 4.250 to 8.465 and dose-normalized AUClast and AUC_{∞} values were increased with increasing dose, ranging from 3.476 to 9.480 and from 3.605 to 9.550, respectively. These results suggest that exposure of TAK-935 increased more than dose proportionally with increasing TAK-935 dose from 200 to 1200 mg.

TABLE 41a
Summary of Plasma Pharmacokinetic Parameters of TAK-935 (Mean
and % CV) Following a Single Dose (Cohort 1, 2, and 3)

| | TAK-935 | | TAK-935 | | TAK-935 | |
|---|---|---|---|---|---|---|
| PK Parameter (unit) | 200 mg | | 600 mg | | 1200 mg | |
| Cₘₐₓ (ng/mL) | 850.0 | (139.3) | 3282 | (49.4) | 10150 | (47.9) |
| tₘₐₓ (h) ^{a} | 0.7500 | (0.267-1.00) | 0.5000 | (0.500-1.50) | 0.5000 | (0.500-1.50) |
| AUCₗₐₛₜ (h*ng/mL) | 695.2 | (90.0) | 3857 | (49.4) | 11390 | (44.3) |
| AUC_{∞} (h*ng/mL) | 720.2 | (88.3) | 3917 | (48.6) | 11450 | (44.1) |
| AUC₂₄ (h*ng/mL) | 698.3 | (89.4) | 3857 | (49.4) | 11290 | (42.8) |
| t_{1/2} [symbol] (h) | 5.888 | (42.3) | 8.695 | (135.2) | 5.075 | (72.4) |
| Lambda z (1/h) | 0.1608 | (82.6) | 0.1634 | (55.6) | 0.1922 | (52.3) |
| CL/F (L/h) | 470.8 | (67.1) | 178.4 | (36.6) | 119.8 | (35.9) |
| V_{z}/F (L) | 4264 | (81.4) | 2337 | (145.1) | 769.2 | (48.4) |

AUCₗₐₛₜ: area under the plasma concentration-time curve from time 0 to time of the last quantifiable concentration; AUC_{∞}: AUC from time 0 to time of infinity; AUC₂₄: AUC from time 0 to 24 hours; CL/F: apparent clearance after extravascular administration; Cₘₐₓ: maximum observed plasma concentration; PK: pharmacokinetic; t_{1/2} [symbol] : terminal disposition phase half-life; tₘₐₓ: time to Cₘₐₓ; V_{z}/F: apparent volume of distribution during the terminal disposition phase after extravascular administration; % CV: percent coefficient of variation.
N = 6 subjects in each dose group
^{a} Median (min-max) [symbol]
indicates data missing or illegible when filed

TABLE 41b
Summary of Dose-Normalized Plasma Pharmacokinetic
Parameters of TAK-935 (Mean and % CV) Following
a Single Dose (Cohort 1, 2, and 3)

| | TAK-935 | TAK-935 | TAK-935 |
|---|---|---|---|
| PK Parameter (unit) | 200 mg | 600 mg | 1200 mg |
| Cₘₐₓ/D (ng/mL/mg) | 4.250 (139.3) | 5.473 (49.5) | 8.465 (47.9) |
| AUCₗₐₛₜ/D (h*ng/mL/mg) | 3.476 (89.9) | 6.432 (49.6) | 9.480 (44.2) |
| AUC [symbol] /D (h*ng/mL/mg) | 3.605 (88.3) | 6.537 (48.6) | 9.550 (44.1) |

AUCₗₐₛₜ: area under the plasma concentration-time curve from time 0 to time of the last quantifiable concentration; AUC_{∞}: AUC from time 0 to time of infinity; Cₘₐₓ: maximum observed plasma concentration; PK: pharmacokinetic; % CV: percent coefficient of variation.
N = 6 subjects in each dose group [symbol]
indicates data missing or illegible when filed

Plasma Concentrations and Pharmacokinetic Parameter Results of TAK-935 (Cohort 4) A summary of plasma PK parameters of TAK-935 following single and multiple doses is presented in Table 41c. A summary of dose-normalized plasma PK parameters of TAK-935 following multiple doses is presented in Table 41d.

On Day 1, following the first dose of TAK-935 100 mg BID, mean Cₘₐₓ values were 254.7 ng/mL. Mean AUCt values were 231.8 h*ng/ml. Median Tₘₐₓ value was 0.7500 hour with a range (min-max) of 0.250 to 2.00 hours. Cₘₐₓ and AUCt values had intersubject variability (% CV) of 86.0% and 53.0%, respectively. Mean t1/2z value was 5.988 hours. On Day 7, Day 14, and Day 21, following multiple doses of TAK-935 100 mg BID, 200 mg BID, and 300 mg BID, mean Cₘₐₓ values were 203.7, 980.3, and 2063 ng/mL, respectively. Mean AUCτ values were 224.4, 860.8, and 2018 h*ng/ml, respectively. Median Tₘₐₓ values were 0.5000 hour with a range (min-max) of 0.250 to 2.00 hours. Cₘₐₓ and AUCτ values had intersubject variability (% CV) of 39.8% to 86.7% and 25.2% to 48.8%, respectively. Mean t1/2z values were 3.630, 2.620, and 2.927 hours, respectively.

Following multiple dose of TAK-935 100 mg BID, 200 mg BID, and 300 mg BID, dose-normalized Cₘₐₓ and AUCτ values were increased with increasing dose, ranging from 2.037 to 6.883 and from 2.244 to 6.730, respectively. These results suggest that exposure of TAK-935 increased more than dose proportionally with increasing TAK-935 dose from 100 mg BID to 300 mg BID.

Accumulation of TAK-935 by multiple doses with 100 mg BID was none to minimum with accumulation ratios for Cₘₐₓ and AUC of 1.047 and 1.054, respectively.

TABLE 41c
Summary of Plasma Pharmacokinetic Parameters of TAK-935 (Mean
and % CV) Following Single and Multiple Doses (Cohort 4)

| PK Parameter | | TAK-935 | | TAK-935 | | TAK-935 | |
|---|---|---|---|---|---|---|---|
| (unit) | | 100 mg BID | | 200 mg BID | | 300 mg BID | |
| Cₘₐₓ (ng/mL) | Day 1 | 254.7 | (86.0) | — | | — | |
| | Day 7, 14, or 21 | 203.7 | (86.7) | 980.3 | (54.8) | 2063 | (39.8) |
| tₘₐₓ (h) ^{a} | Day 1 | 0.7500 | (0.250-2.00) | — | | — | |
| | Day 7, 14, or 21 | 0.5000 | (0.500-2.00) | 0.5000 | (0.500-1.00) | 0.5000 | (0.250-1.00) |
| AUCₗₐₛₜ (h*ng/mL) | Day 1 | 231.8 | (53.0) | — | | — | |
| | Day 7, 14, or 21 | 223.8 | (49.3) | 860.3 | (45.0) | 2018 | (25.2) |
| AUC [symbol] (h*ng/mL) | Day 1 | 231.8 | (53.0) | — | | — | |
| | Day 7, 14, or 21 | 224.4 | (48.8) | 860.8 | (45.0) | 2018 | (25.2) |
| t_{1/2} [symbol] (h) | Day 1 | 5.988 | (101.0) | — | | — | |
| | Day 7, 14, or 21 | 3.630 | (52.5) | 2.620 | (23.9) | 2.927 | (44.6) |
| Lambda z (1/h) | Day 1 | 0.2042 | (59.9) | — | | — | |
| | Day 7, 14, or 21 | 0.2325 | (46.3) | 0.2775 | (24.6) | 0.2708 | (34.0) |
| CL/F (L/h) | Day 1 | 451.8 | (35.5) | — | | — | |
| | Day 7, 14, or 21 | 551.8 | (53.5) | 276.7 | (44.6) | 158.0 | (29.2) |
| V [symbol] /F (L) | Day 1 | 4099 | (104.4) | — | | — | |
| | Day 7, 14, or 21 | 2940 | (65.3) | 1088 | (58.3) | 640.5 | (37.5) |

| | | | | | |
|---|---|---|---|---|---|
| R_{ac(AUC)} | Day 7 | 1.054 | (43.2) | — | — |
| R_{ac(Cmax)} | Day 7 | 1.047 | (76.9) | — | — |

AUCₗₐₛₜ: area under the plasma concentration-time curve from time 0 to time of the last quantifiable concentration; AUC [symbol] : AUC during a dosing interval; BID: twice daily; CL/F: apparent clearance after extravascular administration; Cₘₐₓ: maximum observed plasma concentration; R_{ac(AUC)}: Accumulation ratio based on AUC; R_{ac(Cmax)}: Accumulation ratio based on Cₘₐₓ; PK: pharmacokinetic; t_{1/2} [symbol] : terminal disposition phase half-life; tₘₐₓ: time to Cₘₐₓ; V_{z}/F: apparent volume of distribution during the terminal disposition phase after extravascular administration; % CV: percent coefficient of variation.
N = 6 subjects for TAK-935 group.
—: Not applicable
^{a} Median (min-max) [symbol]
indicates data missing or illegible when filed

TABLE 41d
Summary of Dose-Normalized Plasma Pharmacokinetic Parameters of
TAK-935 (Mean and % CV) Following Multiple Doses (Cohort 4)

| | | TAK-935 | TAK-935 | TAK-935 |
|---|---|---|---|---|
| PK Parameter (unit) | | 100 mg BID | 200 mg BID | 300 mg BID |
| Cₘₐₓ/D (ng/mL/mg) | Day 7, 14, or 21 | 2.037 (86.7) | 4.903 (54.7) | 6.883 (39.9) |
| AUₗₐₛₜ/D (h*ng/mL/mg) | Day 7, 14, or 21 | 2.238 (49.3) | 4.298 (44.9) | 6.725 (25.2) |
| AUC [symbol] /D (h*ng/mL/mg) | Day 7, 14, or 21 | 2.244 (48.8) | 4.302 (44.9) | 6.730 (25.2) |

AUCₗₐₛₜ: area under the plasma concentration-time curve from time 0 to time of the last quantifiable concentration; AUC [symbol] : AUC during a dosing interval; BID: twice daily; Cₘₐₓ: maximum observed plasma concentration; PK: pharmacokinetic; % CV: percent coefficient of variation.
N = 6 subjects for TAK-935 group. [symbol]
indicates data missing or illegible when filed

A summary of plasma PK parameters of M-I following a single dose of TAK-935 is presented in Table 41e. A summary of dose-normalized plasma PK parameters of M-I following a single dose of TAK-935 is presented in Table 41f.

Mean Cₘₐₓ for M-I ranged from 178.0 ng/mL for the 200 mg dose of TAK-935 to 871.0 ng/mL for the 1200 mg dose of TAK-935, with median Tₘₐₓ values of 0.7500 to 1.000 hour. Mean AUC_{∞} values for M-I ranged from 324.8 h*ng/ml for the 200 mg dose to 2408 h*ng/mL for the 1200 mg dose. Mean t1/2z values for M-I ranged from 3.435 to 5.112 hours. Mean MR (based on AUC_{∞}) generally decreased with increasing dose, ranging from 0.5425 to 0.2053, consistent with the greater than dose-proportional increase of TAK 935 exposure with increasing dose.

Following a single dose of TAK-935 200, 600, or 1200 mg, dose-normalized Cₘₐₓ values of M-I were slightly decreased with increasing dose, ranging from 0.8902 to 0.7253, whereas AUClast values of M-I were slightly increased with increasing dose, ranging from 1.589 to 1.995, with similar values observed for AUC_{∞} values of M-I.

TABLE 41e
Summary of Plasma Pharmacokinetic Parameters of M-I (Mean and
% CV) Following a Single Dose of TAK-935 (Cohort 1, 2, and 3)

| | TAK-935 | | TAK-935 | | TAK-935 | |
|---|---|---|---|---|---|---|
| PK Parameter (unit) | 200 mg | | 600 mg | | 1200 mg | |
| Cₘₐₓ (ng/mL) | 178.0 | (68.5) | 531.0 | (13.3) | 871.0 | (40.8) |
| tₘₐₓ (h) ^{a} | 0.7500 | (0.500-1.00) | 1.000 | (0.500-1.50) | 1.000 | (0.500-1.50) |
| AUCₗₐₛₜ (h*ng/mL) | 318.0 | (49.7) | 1075 | (15.6) | 2393 | (40.8) |
| AUC_{∞} (h*ng/mL) | 324.8 | (49.0) | 1087 | (15.8) | 2408 | (40.7) |
| AUC₂₄ (h*ng/mL) | 320.7 | (49.4) | 1077 | (15.3) | 2370 | (39.1) |
| t_{1/2} [symbol] (h) | 3.435 | (87.0) | 5.040 | (88.0) | 5.112 | (36.5) |
| Lambda z (1/h) | 0.2980 | (48.4) | 0.2139 | (54.5) | 0.1508 | (35.6) |
| MR | 0.5425 | (31.4) | 0.3075 | (34.0) | 0.2053 | (11.9) |

AUCₗₐₛₜ: area under the plasma concentration-time curve from time 0 to time of the last quantifiable concentration; AUC_{∞}: AUC from time 0 to time of infinity; AUC₂₄: AUC from time 0 to 24 hours; Cₘₐₓ: maximum observed plasma concentration; MR: metabolic ratio; PK: pharmacokinetic; t_{1/2} [symbol] : terminal disposition phase half-life; tₘₐₓ: time to Cₘₐₓ; % CV: percent coefficient of variation.
N = 6 subjects in each dose group
^{a} Median (min-max) [symbol]
indicates data missing or illegible when filed

TABLE 41f
Summary of Dose-Normalized Plasma Pharmacokinetic
Parameters of M-I (Mean and % CV) Following
a Single Dose of TAK-935 (Cohort 1, 2, and 3)

| PK Parameter | TAK-935 | TAK-935 | TAK-935 |
|---|---|---|---|
| (unit) | 200 mg | 600 mg | 1200 mg |
| Cₘₐₓ/D | 0.8902 (68.5) | 0.8858 (13.4) | 0.7253 (40.7) |
| (ng/mL/mg) | | | |
| AUCₗₐₛₜ/D | 1.589 (49.5) | 1.792 (15.6) | 1.995 (40.8) |
| (h*ng/mL/mg) | | | |
| AUC_{∞}/D | 1.625 (49.1) | 1.810 (15.8) | 2.010 (40.7) |
| (h*ng/mL/mg) | | | |

AUCₗₐₛₜ: area under the plasma concentration-time curve from time 0 to time of the last quantifiable concentration; AUC_{∞}: AUC from time 0 to time of infinity, Cₘₐₓ: maximum observed plasma concentration; PK: pharmacokinetic; % CV: percent coefficient of variation.
N = 6 subjects in each dose group

Plasma Concentrations and Pharmacokinetic Parameter Results of M-I (Cohort 4) A summary of plasma PK parameters of M-I following single and multiple doses of TAK-935 is presented in Table 41g. A summary of dose-normalized plasma PK parameters of M-I following multiple doses of TAK-935 is presented in Table 41g.

Mean Cₘₐₓ for M-I, ranged from 83.83 ng/ml for the 100 mg BID dose of TAK-935 on Day 1 to 371.5 ng/ml for the 300 mg BID dose of TAK-935 on Day 21. Median Tₘₐₓ values ranged from 0.5000 to 1.000 hour across the dose range studied. Mean AUC, values for M-I ranged from 147.4 h*ng/mL for the 100 mg BID dose on Day 7 to 629.5 h*ng/ml for the 300 mg BID dose on Day 21, with similar values for AUClast. Following 100 mg BID of TAK-935, the exposure of M-I was comparable between Day 1 and Day 7. Mean t1/2z values for M-I ranged from 1.842 to 2.462 hours. Mean MR, based on AUCτ, generally decreased with increasing dose, ranging from 0.7375 to 0.3097 after multiple doses (100 mg BID to 300 mg BID), consistent with the greater than dose-proportional increase of TAK 935 exposure with increasing dose.

Following multiple dose of TAK-935 100 mg BID, 200 mg BID, and 300 mg BID, dose-normalized Cₘₐₓ and AUC, values for M-I were increased with increasing dose, ranging from 0.8625 to 1.266 and from 1.474 to 2.100, respectively.

Accumulation of M-I by multiple doses of TAK-935 with 100 mg BID was none to minimum with accumulation ratios for Cₘₐₓ and AUC of 1.247 and 0.9090, respectively.

TABLE 41g
Summary of Plasma Pharmacokinetic Parameters of M-I (Mean and
% CV) Following Single and Multiple Doses of TAK-935 (Cohort 4)

| | | TAK-935 | | TAK-935 | | TAK-935 | |
|---|---|---|---|---|---|---|---|
| PK Parameter (unit) | | 100 mg BID | | 200 mg BID | | 300 mg BID | |
| Cₘₐₓ (ng/mL) | Day 1 | 83.83 | (39.0) | — | | — | |
| | Day 7, 14, or 21 | 86.25 | (46.3) | 252.8 | (43.2) | 371.5 | (25.4) |
| tₘₐₓ (h) ^{a} | Day 1 | 1.000 | (0.500-2.00) | — | | — | |
| | Day 7, 14, or 21 | 0.7500 | (0.500-2.00) | 0.5000 | (0.500-1.00) | 0.5000 | (0.500-1.00) |
| AUCₗₐₛₜ (h*ng/mL) | Day 1 | 163.0 | (24.7) | — | | — | |
| | Day 7, 14, or 21 | 146.5 | (27.2) | 383.5 | (30.7) | 629.3 | (13.9) |
| AUC [symbol] (h*ng/mL) | Day 1 | 163.8 | (24.6) | — | | — | |
| | Day 7, 14, or 21 | 147.4 | (26.9) | 383.5 | (30.7) | 629.5 | (14.0) |
| t_{1/2} [symbol] (h) | Day 1 | 2.150 | (62.1) | — | | — | |
| | Day 7, 14, or 21 | 1.842 | (16.4) | 2.302 | (45.2) | 2.462 | (32.1) |
| Lambda z (1/h) | Day 1 | 0.4022 | (40.5) | — | | — | |
| | Day 7, 14, or 21 | 0.3868 | (19.8) | 0.3353 | (27.6) | 0.3030 | (27.3) |
| MR | Day 1 | 0.7640 | (32.0) | — | | — | |
| | Day 7, 14, or 21 | 0.7375 | (46.4) | 0.4615 | (27.3) | 0.3097 | (19.2) |

| | | | | | |
|---|---|---|---|---|---|
| R_{ac(AUC)} | Day 7 | 0.9090 | (21.6) | — | — |
| R_{ac(Cmax)} | Day 7 | 1.247 | (73.5) | — | — |

AUCₗₐₛₜ: area under the plasma concentration-time curve from time 0 to time of the last quantifiable concentration; AUC [symbol] : AUC during a dosing interval: BID: twice daily; Cₘₐₓ: maximum observed plasma concentration; MR: metabolic ratio; PK: pharmacokinetic; R_{ac(AUC)}: Accumulation ratio based on AUC; R_{ac(Cmax)}: Accumulation ratio based on Cₘₐₓ; t_{1/2} [symbol] : terminal disposition phase half-life; tₘₐₓ: time to Cₘₐₓ; % CV: percent coefficient of variation.
N = 6 subjects for TAK-935 group.
—: Not applicable
^{a} Median (min-max) [symbol]
indicates data missing or illegible when filed

### Plasma Pharmacodynamic Results (Cohort 1, 2, and 3)

Descriptive statistics for plasma PD parameter estimates of 24HC are summarized in Table 41h for Baseline (Day-1) and in Table 41i after a single oral dose of TAK-935 or placebo. A summary of percent change from Baseline in PD parameters of plasma 24HC following a single dose is presented in Table 41j.

At Baseline (Day-1), mean plasma 24HC concentrations were similar across the placebo and TAK-935 groups. Mean plasma 24HC concentrations generally fluctuated in the range of 40 to 60 ng/mL across all dose groups.

After TAK-935 single-dose administration, a trend of decreased plasma 24HC concentrations was observed on Day 1 compared with the time-matched baseline values on Day-1 in all the TAK-935 dose groups. However, a clear dose dependency of decrease in plasma 24HC concentrations was not observed on Day 1. The degree of 24HC decrease on Days 1 to 3 postdose appeared to increase with increasing dose up to 1200 mg. For all dose groups, the maximum decrease in plasma 24HC concentrations was observed at approximately 48 hours postdose. The maximum mean decrease of plasma 24HC concentrations from time-matched baseline at approximately 48 hours postdose in the TAK-935 1200 mg dose group was about 38%.

At Baseline (Day-1), no apparent difference in AUEC₂₄, Emax, E24 or time to Emax was observed across all dose groups, including the placebo group. After TAK-935 single-dose administration, a weak trend of decreasing AUEC₂₄ and Emax was observed with increasing TAK-935 dose. When compared with the placebo group, the TAK-935 1200 mg dose group showed a maximum decrease of approximately 11.4% in percent change from Baseline of mean AUEC₂₄.

TABLE 41h
Summary of Pharmacodynamic Parameters of Plasma 24HC (Mean
and % CV) at Baseline (Day −1) (Cohort 1, 2, and 3)

| | | | | Time |
|---|---|---|---|---|
| Dose | AUEC₂₄ | Eₘₐₓ | E₂₄ | to Eₘₐₓ |
| Group | (h*ng/mL) | (ng/mL) | (ng/mL) | (h) ^{a} |
| Placebo | 1157 (27.1) | 45.05 (30.8) | 50.83 (25.2) | 4.500 (0.00-12.0) |
| 200 mg | 1116 (26.5) | 43.57 (29.9) | 50.12 (22.8) | 8.000 (1.00-12.0) |
| 600 mg | 1432 (31.0) | 54.23 (30.2) | 64.08 (31.4) | 6.500 (0.00-12.0) |
| 1200 mg | 1013 (14.7) | 38.48 (16.4) | 43.12 (13.5) | 2.000 (0.00-4.00) |

AUEC₂₄: area under the effect-time curve from time 0 to 24 hours; Eₘₐₓ: maximum drug-induced effect; E₂₄: observed effect at 24 hours; % CV: percent coefficient of variation; 24HC; 24S-hydroxycholesterol.
N = 6 subjects for Placebo group, N = 6 subjects for each TAK-935 dose group.
^{a} Median (min-max)

TABLE 41i
Summary of Pharmacodynamic Parameters of Plasma 24HC (Mean and
% CV) Following a Single Dose (Day 1) (Cohort 1, 2, and 3)

| | AUEC₂₄ | AUEC [symbol] | Eₘₐₓ | E₂₄ | Time to Eₘₐₓ |
|---|---|---|---|---|---|
| Dose Group | (h*ng/mL) | (h*ng/mL) | (ng/mL) | (ng/mL) | (h) ^{a} |
| Placebo | 1191 (27.3) | 2403 (26.9) | 45.38 (28.2) | 50.78 (24.8) | 12.00 (8.00-12.0) |
| 200 mg | 1017 (27.5) | 1918 (26.0) | 35.25 (24.3) | 39.95 (25.4) | 48.00 (48.0-48.0) |
| 600 mg | 1311 (30.2) | 2425 (31.2) | 43.25 (35.3) | 48.47 (32.0) | 48.00 (24.0-48.0) |
| 1200 mg | 898.3 (16.6) | 1627 (18.7) | 27.30 (24.4) | 33.57 (19.8) | 48.00 (48.0-48.0) |

AUEC [symbol] : area under the effect-time curve from time 0 to last sampling time point (t); AUEC₂₄: AUEC from time 0 to 24 hours; Eₘₐₓ: maximum drug-induced effect; E₂₄: observed effect at 24 hours; % CV: percent coefficient of variation; 24HC: 24S-hydroxycholesterol.
N = 6 subjects for Placebo group, N = 6 subjects for each TAK-935 dose group.
^{a} Median (min-max) [symbol]
indicates data missing or illegible when filed

TABLE 41j
Summary of % Change From Baseline in Pharmacodynamic
Parameters of Plasma 24HC (Mean and % CV) Following
a Single Dose (Cohort 1, 2, and 3)

| Dose Group | AUEC₂₄ (%) | | Eₘₐₓ (%) | | E₂₄ (%) | |
|---|---|---|---|---|---|---|
| Placebo | 2.790 | (188.6) | 1.640 | (350.2) | −0.1650 | (−2875) |
| 200 mg | −8.875 | (−46.5) | −17.69 | (−40.4) | −20.29 | (−45.9) |
| 600 mg | −8.482 | (−37.6) | −21.40 | (−38.2) | −24.02 | (−28.1) |
| 1200 mg | −11.36 | (−40.8) | −29.67 | (−32.2) | −22.65 | (−34.5) |

AUEC₂₄: area under the effect-time curve from time 0 to 24 hours; Eₘₐₓ: maximum drug-induced effect; E₂₄: observed effect at 24 hours; % CV: percent coefficient of variation; 24HC: 24S-hydroxycholesterol.
N = 6 subjects for Placebo group, N = 6 subjects for each TAK-935 dose group.

### Plasma Pharmacodynamic Results (Cohort 4)

Descriptive statistics for plasma PD parameter estimates of 24HC are summarized in Table 41k for Baseline (Day-1) and in Table 411 after multiple oral doses of TAK-935 or placebo. A summary of percent change from Baseline in PD parameters of plasma 24HC following multiple doses is presented in Table 41m.

At Baseline (Day-1), mean plasma 24HC concentrations generally fluctuated in the range of 37 to 45 ng/mL in both placebo and TAK-935 groups.

After TAK-935 multiple-dose administration, mean plasma 24HC concentrations at trough time points showed an apparent dose-dependent decrease with more profound decreases at higher doses. On Day 21, the maximum mean decrease of trough plasma 24HC concentrations from time-matched baseline was about 88% in the TAK-935 group. At Baseline (Day-1), no apparent difference in AUEC₂₄, AUECτ, Emax, E₁₂, E₂₄ or time to Emax was observed in both placebo and TAK-935 group. After TAK-935 multiple-dose administration, an apparent decrease of AUEC₂₄, AUECτ, Emax, E₁₂, and E₂₄ was observed on Day 21. When compared with the placebo group, the TAK-935 group showed a maximum decrease of approximately 86.8% in percent change from Baseline of mean AUEC₂₄.

TABLE 41k
Summary of Pharmacodynamic Parameters of Plasma 24HC
(Mean and % CV) at Baseline (Day −1) (Cohort 4)

| | | | | | | Time |
|---|---|---|---|---|---|---|
| | AVEC₂₄ | AUEC_{τ} | Eₘₐₓ | E₁₂ | E₂₄ | to Eₘₐₓ |
| Dose Group | (h*ng/mL) | (h*ng/mL) | (ng/mL) | (ng/mL) | (ng/mL) | (h) ^{a} |
| Placebo | 1004 | 491.3 | 37.03 | 40.37 | 44.77 | 8.000 |
| | (16.1) | (13.7) | (11.4) | (21.7) | (15.4) | (4.00- |
| | | | | | | 12.0) |
| 100 mg BID, 200 mg BID, and 300 mg BID | 973.3 | 473.8 | 35.68 | 41.05 | 42.22 | 8.000 |
| | (22.3) | (21.6) | (18.9) | (24.3) | (22.5) | (4.00- |
| | | | | | | 8.00) |

AUEC₂₄: area under the effect-time curve from time 0 to 24 hours; AUEC_{τ}: AUEC during a dosing interval; BID: twice daily; Eₘₐₓ: maximum drug-induced effect; E₁₂: observed effect at 12 hours; E₂₄: observed effect at 24 hours; % CV: percent coefficient of variation; 24HC: 24S-hydroxycholesterol.
N = 3 subjects for Placebo group, N = 6 subjects for TAK-935 group.
^{a} Median (min-max)

TABLE 41l
Summary of Pharmacodynamic Parameters of Plasma 24HC (Mean
and % CV) Following Multiple Doses (Day 21) (Cohort 4)

| | | | | | | Time |
|---|---|---|---|---|---|---|
| | AUEC₂₄ | AVEC_{τ} | Eₘₐₓ | E₁₂ | E₂₄ | to Eₘₐₓ |
| Dose Group | (h*ng/mL) | (h*ng/mL) | (ng/mL) | (ng/mL) | (ng/mL) | (h) ^{a} |
| Placebo | 1007 | 497.7 | 38.53 | 43.23 | 41.77 | 1.000 |
| | (20.2) | (18.6) | (20.2) | (21.8) | (22.3) | (1.00- |
| | | | | | | 4.00) |
| 100 mg BID, 200 mg BID, and 300 mg BID | 124.2 | 61.28 | 4.640 | 5.443 | 5.013 | 6.000 |
| | (12.2) | (12.4) | (16.0) | (12.3) | (16.1) | (1.00- |
| | | | | | | 8.00) |

AUEC₂₄: area under the effect-time curve from time 0 to 24 hours; AUEC_{τ}: AUEC during a dosing interval; BID: twice daily; Eₘₐₓ: maximum drug-induced effect; E₁₂: observed effect at 12 hours; E₂₄: observed effect at 24 hours; % CV: percent coefficient of variation; 24HC: 24S-hydroxycholesterol.
N = 3 subjects for Placebo group, N = 6 subjects for TAK-935 group.
^{a} Median (min-max)

TABLE 41m
Summary of % Change From Baseline in Pharmacodynamic
Parameters of Plasma 24HC (Mean and % CV)
Following Multiple Doses (Cohort 4)

| | AUEC₂₄ | AUEC [symbol] | Eₘₐₓ | E₁₂ | E₂₄ |
|---|---|---|---|---|---|
| Dose Group | (%) | (%) | (%) | (%) | (%) |
| Placebo | 0.1567 | 0.9167 | 3.409 | 7.107 | −6.976 |
| | (3608.1) | (630.1) | (254.6) | (32.6) | (−156.3) |
| 100 mg BID, | −86.75 | −86.62 | −86.72 | −85.83 | −87.67 |
| 200 mg BID, | (−3.6) | (−3.2) | (−2.8) | (−5.6) | (−3.3) |
| and 300 mg BID | | | | | |

AUEC₂₄: area under the effect-time curve from time 0 to 24 hours; AUEC [symbol] : AUEC during a dosing interval; BID: twice daily; Eₘₐₓ: maximum drug-induced effect; E₁₂: observed effect at 12 hours; E₂₄: observed effect at 24 hours; % CV: percent coefficient of variation; 24HC: 24S-hydroxycholesterol.
N = 3 subjects for Placebo group, N = 6 subjects for TAK-935 group. [symbol]
indicates data missing or illegible when filed

### Pharmacokinetic Conclusions

### Cohort 1, 2, and 3

After single-dose oral administration of TAK-935 200, 600, and 1200 mg in healthy Japanese subjects, TAK 935 Cₘₐₓ was reached rapidly at 0.5000 to 0.7500 hour postdose (median Tₘₐₓ). Mean TAK-935 t1/2z ranged from 5.075 to 8.695 hours. Exposure to TAK-935 increased in a greater than dose-proportional manner over the 200 to 1200 mg dose range. Following a single dose of TAK-935 200, 600, or 1200 mg, dose-normalized Cₘₐₓ values were increased with increasing dose, ranging from 4.250 to 8.465 and dose-normalized AUClast and AUC_{∞} value ere increased with increasing dose, ranging from 3.476 to 9.480 and from 3.605 to 9.550, respectively, indicating greater than dose proportional increase in the exposure of TAK-935.

After single-dose oral administration of TAK-935 200, 600, and 1200 mg in healthy Japanese subjects, metabolite M-I showed median Tₘₐₓ values of 0.7500 to 1.000 hour and mean t1/2z values of 3.435 to 5.112 hours. Mean MR (based on AUC.) generally decreased with increasing dose, ranging from 0.5425 to 0.2053, consistent with the greater than dose-proportional increase of TAK 935 exposure with increasing dose.

### Cohort 4

After multiple-dose administrations with up-titration of TAK-935 starting at 100 mg BID, then at 200 mg BID, and finally at 300 mg BID in healthy Japanese subjects, TAK-935 Cₘₐₓ was reached rapidly at 0.7500 hour post dose (median Tₘₐₓ) on Day 1. Mean t1/2z value were 5.988 hours on Day 1, and 3.630, 2.620, and 2.927 hours on Day 7, 14, and 21, respectively. Following multiple dose of TAK-935 100 mg BID, 200 mg BID, and 300 mg BID, dose-normalized Cₘₐₓ and AUC, values were increased with increasing dose, ranging from 2.037 to 6.883 and from 2.244 to 6.730, respectively, indicating greater than dose proportional increase in the exposure of TAK-935. Accumulation of TAK-935 by multiple doses with 100 mg BID was none to minimum with accumulation ratios for Cₘₐₓ and AUC of 1.047 and 1.054, respectively.

M-I metabolite showed median Tₘₐₓ values ranged from 0.5000 to 1.000 hour across the dose range studied. Mean t_{1/2z} values for M-I ranged from 1.842 to 2.462 hours. Following 100 mg BID of TAK 935, the exposure of M-I was comparable between Day 1 and Day 7. Mean MR, based on AUC_{τ}, generally decreased with increasing dose, ranging from 0.7375 to 0.3097 after multiple doses (100 mg BID to 300 mg BID), consistent with the greater than dose-proportional increase of TAK 935 exposure with increasing dose.

### Pharmacodynamic Conclusions

### Cohort 1, 2, and 3

After single-dose oral administration of TAK-935 200, 600, and 1200 mg, the degree of decrease in plasma 24HC concentrations generally increased with increasing dose. When compared with the placebo group, the TAK-935 1200 mg dose group showed a maximum decrease of approximately 11.4% in percent change from Baseline of mean AUEC₂₄.

### Cohort 4

After multiple-dose administrations with up-titration of TAK-935 starting at 100 mg BID, then at 200 mg BID, and finally at 300 mg BID, a generally dose-dependent decrease in plasma 24HC concentrations was observed, with more profound decreases at higher doses. When compared with the placebo group, the TAK-935 group showed a maximum decrease of approximately 86.8% in percent change from Baseline of mean AUEC₂₄.

### DISCUSSION

This study was designed to evaluate the safety, tolerability, PK, and PD of single and multiple dosing with up-titration of TAK-935 in healthy Japanese subjects.

In this study, a single oral dose of TAK-935 up to 1200 mg and multiple doses with up-titration from 100 mg BID to 300 mg BID of TAK-935 in healthy Japanese subjects were well tolerated with no new safety concerns. All of the TEAEs were mild. No deaths, SAEs, or TEAEs leading to study drug discontinuation were reported during the study. Following multiple doses with up-titration of TAK-935 starting at 100 mg BID, then at 200 mg BID, and finally at 300 mg BID, 5 subjects (83.3%) in the TAK-935 group experienced TEAEs. However, all of the TEAEs were mild in intensity, and no TEAEs were reported in more than 1 subject. Drug-related TEAEs were reported in 3 subjects (50.0%) (diarrhoea, hiccups, and rash). All the TEAEs were recovered/resolved, except alopecia areata. There were no clinically meaningful differences between TAK-935 and placebo in safety laboratory, vital signs and weight, or ECG assessments.

In all treatments, TAK-935 was rapidly absorbed after the administration with Tₘₐₓ ranged from 0.5000 to 0.7500 hour (median Tₘₐₓ). TAK 935 appeared to have fast elimination with mean t_{1/2z} values ranged from 5.075 to 8.695 hours after a single dose and from 2.927 to 3.630 hours after multiple doses with up-titration. Dose-normalized Cₘₐₓ and AUC increased with increasing dose, ranging from 4.250 to 8.465 and from 3.605 to 9.550 after a single dose and ranging from 2.037 to 6.883 and from 2.244 to 6.730 after multiple doses with up-titration, respectively.

Collectively, exposure to TAK-935 increased in a greater than dose-proportional manner. In addition, multiple dose administration resulted in none to minimum accumulation of Cₘₐₓ and AUC of TAK-935 at <6%.

M-I rapidly reached peak plasma concentrations at approximately 0.5000 to 1.000 hour postdose (median Tₘₐₓ), shortly after the TAK-935 median Tₘₐₓ of 0.5000 to 0.7500 hour postdose. Mean t_{1/2z} values for M-I ranged from 3.435 to 5.112 hours after a single dose and from 1.842 to 2.462 hours after multiple doses, respectively. These values were generally shorter than the mean t_{1/2z} values of the parent drug TAK-935. MR (based on AUC. or AUC_{τ}) generally decreased with increasing dose (ranging from 0.5425 to 0.2053 after a single dose and from 0.7375 to 0.3097 after multiple doses with up-titration), consistent with the greater than dose-proportional increase of TAK-935 exposure with increasing dose. The data suggested that there were no significant differences in TAK-935 exposure and pharmacokinetics between Japanese and non-Japanese subjects are similar. The PD marker of plasma 24HC concentration was measured at matched time points on Day-1 predose (Baseline) and Day 1 postdose (over 24 hours for each day) and at additional time points up to Day 3 following a single dose of TAK-935, and at matched time points on Day-1 predose (Baseline) and Day 21 postdose and at additional trough time points following multiple doses of TAK-935. The time-matched plasma 24HC measurements allowed the evaluation of the drug effect on 24HC concentration without potential influence of circadian rhythm. After TAK-935 single-dose administration, the degree of decrease in plasma 24HC concentrations generally increased with increasing dose. For all TAK-935 dose groups, the nadir was observed as late as 48 hours postdose, which is considerably delayed compared with the median Tₘₐₓ of 0.5000 to 0.7500 hours of TAK-935 in plasma. Time-matched percent change from Baseline in 24HC concentration (Days 1 to 3 vs Day-1) showed a maximum decrease of approximately 38% at 48 hours postdose for the 1200 mg dose group. Similarly, time-matched percent change from Baseline showed a maximum decrease of approximately 11.4% for AUEC₂₄, again for the 1200 mg dose group. The maximum decrease in plasma 24HC concentrations was observed as late as 48 hours across all dose groups; therefore, data from the time-matched 24-hour sampling period should be interpreted with some caution. After multiple-dose administrations with up-titration of TAK-935, a generally dose-dependent decrease in plasma 24HC concentrations was observed, with more profound decreases at higher doses. On Day 21, time-matched percent change from Baseline in trough 24HC concentration (Day 21 vs Day-1) showed a maximum decrease about 88% in the TAK-935 group. Time-matched percentage change from Baseline on Day 21 showed a maximum decrease of approximately 86.8% for AUEC₂₄ in the TAK-935 group.

### CONCLUSIONS

Single and multiple doses with up-titration of TAK-935 in healthy Japanese subjects were well tolerated with no new safety concerns.

TAK-935 was rapidly absorbed at all doses examined in this study and exposure increased in a greater than dose-proportional manner with increasing dose. Mean MR, based on AUC_{∞} or AUC_{τ}, generally decreased with increasing dose, from 0.5425 to 0.2053 after a single dose and from 0.7375 to 0.3097 after multiple doses with up-titration.

After TAK-935 single-dose administration and multiple-dose administrations with up-titration of TAK-935, the degree of decrease in plasma 24HC concentrations generally increased with increasing dose. Time-matched AUEC₂₄ decreased from Baseline by approximately 11.4% and 86.8% for the TAK-935 1200 mg dose group after a single dose and for the TAK-935 group after multiple-dose administrations with up-titration, respectively.

Further data from the study is provided below.

TABLE 42
Summary of Pharmacokinetic Parameters of TAK-935
Day 1
Part 2
<Pharmacokinetic Analysis Set>
Analyte: TAK-935

| | | Statistics | | | | | | | | | | | |
|---|---|---|---|---|---|---|---|---|---|---|---|---|---|
| | | | | N of Geo | | | | | | | | | |
| Variable | Dose | N | Mean | Mean | Geo Mean | SD | CV | Geo CV | Minimum | Q1 | Median | Q3 | Maximum |
| Cₘₐₓ (ng/mL) | TAK-935 | 6 | 254.7 | 6 | 201.0 | 219.13 | 86.0 | 79.7 | 105 | 137.0 | 146.0 | 320.0 | 674 |
| AUC1ast (h*ng/mL) | TAK-935 | 6 | 231.8 | 6 | 209.0 | 122.95 | 53.0 | 51.0 | 139 | 158.0 | 160.0 | 348.0 | 426 |
| AUCtau (h*ng/mL) | TAK-935 | 6 | 231.8 | 6 | 209.0 | 122.95 | 53.0 | 51.0 | 139 | 158.0 | 160.0 | 348.0 | 426 |

TABLE 43
Summary of Pharmacokinetic Parameters of TAK-935
Day 1
Part 2
<Pharmacokinetic Analysis Set>
Analyte: TAK-935

| | | Statistics | | | | | | | |
|---|---|---|---|---|---|---|---|---|---|
| Variable | Dose | N | Mean | SD | Minimum | Q1 | Median | Q3 | Maximum |
| Tₘₐₓ (h) | TAK-935 | 6 | 0.8750 | 0.62750 | 0.250 | 0.5000 | 0.7500 | 1.000 | 2.00 |

TABLE 44
Summary of Pharmacokinetic Parameters of TAK-935
Day 1
Part 2
<Pharmacokinetic Analysis Set>
Analyte: TAK-935

| Variable | Dose | N | Mean | SD | CV | Minimum | Q1 | Median | Q3 | Maximum |
|---|---|---|---|---|---|---|---|---|---|---|
| T1/2z (h) | TAK-935 | 6 | 5.988 | 6.0512 | 101.0 | 2.01 | 2.140 | 3.275 | 7.630 | 17.6 |
| Lambda z (1/h) | TAK-935 | 6 | 0.2042 | 0.12237 | 59.9 | 0.0395 | 0.09090 | 0.2135 | 0.3240 | 0.344 |
| CL/F (L/H) | TAK-935 | 6 | 451.8 | 160.28 | 35.5 | 231 | 280.0 | 501.5 | 580.0 | 617 |
| Vz/F (L) | TAK-935 | 6 | 4099 | 4279.9 | 104.4 | 671 | 1200 | 2450 | 5820 | 12000 |

TABLE 45
Summary of Pharmacokinetic Parameters of TAK-935
Day 7
Part 2
<Pharmacokinetic Analysis Set>
Analyte: TAK-935

| | | Statistics | | | | | | | | | | | |
|---|---|---|---|---|---|---|---|---|---|---|---|---|---|
| | | | | N of Geo | | | | | | | | | |
| Variable | Dose | N | Mean | Mean | Geo Mean | SD | CV | Geo CV | Minimum | Q1 | Median | Q3 | Maximum |
| Cₘₐₓ (ng/mL) | TAK-935 | 6 | 203.7 | 6 | 146.5 | 176.64 | 86.7 | 113.1 | 49.1 | 65.20 | 148.5 | 302.0 | 509 |
| AUC1ast (h*ng/mL) | TAK-935 | 6 | 223.8 | 6 | 201.3 | 110.24 | 49.3 | 55.4 | 91.9 | 139.0 | 226.0 | 248.0 | 412 |
| AUCtau (h*ng/mL) | TAK-935 | 6 | 224.4 | 6 | 202.3 | 109.6 | 48.8 | 54.6 | 93.3 | 141.0 | 226.0 | 248.0 | 412 |

TABLE 46
Summary of Pharmacokinetic Parameters of TAK-935
Day 7
Part 2
<Pharmacokinetic Analysis Set>
Analyte: TAK-935

| | | Statistics | | | | | | | |
|---|---|---|---|---|---|---|---|---|---|
| Variable | Dose | N | Mean | SD | Minimum | Q1 | Median | Q3 | Maximum |
| Tₘₐₓ (h) | TAK-935 | 6 | 0.8333 | 0.60553 | 0.500 | 0.5000 | 0.5000 | 1.000 | 2.00 |

TABLE 47
Summary of Pharmacokinetic Parameters of TAK-935
Day 7
Part 2
<Pharmacokinetic Analysis Set>
Analyte: TAK-935

| Variable | Dose | N | Mean | SD | CV | Minimum | Q1 | Median | Q3 | Maximum |
|---|---|---|---|---|---|---|---|---|---|---|
| T1/2z (h) | TAK-935 | 6 | 3.630 | 1.9050 | 52.5 | 1.68 | 2.550 | 3.270 | 3.880 | 7.13 |
| Lambda z (1/h) | TAK-935 | 6 | 0.2325 | 0.10759 | 46.3 | 0.0972 | 0.1790 | 0.2180 | 0.2710 | 0.412 |
| CL/F (L/H) | TAK-935 | 6 | 551.8 | 294.98 | 53.5 | 243 | 403.0 | 443.0 | 709.0 | 1070 |
| Vz/F (L) | TAK-935 | 6 | 2940 | 1918.3 | 65.3 | 588 | 1590 | 2595 | 4370 | 5900 |

TABLE 49
Summary of Pharmacokinetic Parameters of TAK-935
Day 14
Part 2
<Pharmacokinetic Analysis Set>
Analyte: TAK-935

| | | Statistics | | | | | | | | | | | |
|---|---|---|---|---|---|---|---|---|---|---|---|---|---|
| | | | | N of Geo | | | | | | | | | |
| Variable | Dose | N | Mean | Mean | Geo Mean | SD | CV | Geo CV | Minimum | Q1 | Median | Q3 | Maximum |
| Cₘₐₓ (ng/mL) | TAK-935 | 6 | 980.3 | 6 | 835.5 | 536.91 | 54.8 | 73.8 | 319 | 461.0 | 1026 | 1390 | 1660 |
| AUC1ast (h*ng/mL) | TAK-935 | 6 | 860.3 | 6 | 788.0 | 387.58 | 45.0 | 49.0 | 464 | 481.0 | 793.5 | 1230 | 1400 |
| AUCtau (h*ng/mL) | TAK-935 | 6 | 860.8 | 6 | 788.6 | 387.31 | 45.0 | 48.9 | 464 | 482.0 | 794.5 | 1230 | 1400 |

TABLE 48
Summary of Pharmacokinetic Parameters of TAK-935
Day 7
Part 2
<Pharmacokinetic Analysis Set>
Analyte: TAK-935

| Variable | Dose | N | Mean | SD | CV | Minimum | Q1 | Median | Q3 | Maximum |
|---|---|---|---|---|---|---|---|---|---|---|
| Rac (AUC) | TAK-935 | 6 | 1.054 | 0.45584 | 43.2 | 0.553 | 0.5870 | 1.028 | 1.560 | 1.57 |
| Rac (Cₘₐₓ) | TAK-935 | 6 | 1.047 | 0.80523 | 76.9 | 0.154 | 0.3190 | 1.016 | 1.590 | 2.19 |

TABLE 50
Summary of Pharmacokinetic Parameters of TAK-935
Day 14
Part 2
<Pharmacokinetic Analysis Set>
Analyte: TAK-935

| | | Statistics | | | | | | | |
|---|---|---|---|---|---|---|---|---|---|
| Variable | Dose | N | Mean | SD | Minimum | Q1 | Median | Q3 | Maximum |
| Tₘₐₓ (h) | TAK-935 | 6 | 0.6667 | 0.25820 | 0.500 | 0.5000 | 0.5000 | 1.000 | 1.00 |

TABLE 51
Summary of Pharmacokinetic Parameters of TAK-935
Day 14
Part 2
<Pharmacokinetic Analysis Set>
Analyte: TAK-935

| Variable | Dose | N | Mean | SD | CV | Minimum | Q1 | Median | Q3 | Maximum |
|---|---|---|---|---|---|---|---|---|---|---|
| T1/2z (h) | TAK-935 | 6 | 2.620 | 0.62645 | 23.9 | 1.78 | 2.200 | 2.575 | 3.010 | 3.58 |
| Lambda z (1/h) | TAK-935 | 6 | 0.2775 | 0.068196 | 24.6 | 0.194 | 0.2300 | 0.2685 | 0.3150 | 0.389 |
| CL/F (L/H) | TAK-935 | 6 | 276.4 | 123.39 | 44.6 | 143 | 163.0 | 254.0 | 415.0 | 431 |
| Vz/F (L) | TAK-935 | 6 | 1088 | 634.67 | 58.3 | 419 | 540.0 | 1029 | 1370 | 2140 |

TABLE 53
Summary of Pharmacokinetic Parameters of TAK-935
Day 21
Part 2
<Pharmacokinetic Analysis Set>
Analyte: TAK-935

| | | Statistics | | | | | | | |
|---|---|---|---|---|---|---|---|---|---|
| Variable | Dose | N | Mean | SD | Minimum | Q1 | Median | Q3 | Maximum |
| Tₘₐₓ (h) | TAK-935 | 6 | 0.5417 | 0.24580 | 0.250 | 0.5000 | 0.5000 | 0.5000 | 1.00 |

TABLE 52
Summary of Pharmacokinetic Parameters of TAK-935
Day 21
Part 2
<Pharmacokinetic Analysis Set>
Analyte: TAK-935

| | | Statistics | | | | | | | | | | | |
|---|---|---|---|---|---|---|---|---|---|---|---|---|---|
| | | | | N of Geo | | | | | | | | | |
| Variable | Dose | N | Mean | Mean | Geo Mean | SD | CV | Geo CV | Minimum | Q1 | Median | Q3 | Maximum |
| Cₘₐₓ (ng/mL) | TAK-935 | 6 | 2063 | 6 | 1945 | 820.97 | 39.8 | 37.7 | 1470 | 1500 | 1605 | 2850 | 3350 |
| AUC1ast (h*ng/mL) | TAK-935 | 6 | 2018 | 6 | 1960 | 509.17 | 25.2 | 27.7 | 1230 | 1790 | 2045 | 2240 | 2760 |
| AUCtau (h*ng/mL) | TAK-935 | 6 | 2018 | 6 | 1960 | 509.17 | 25.2 | 27.7 | 1230 | 1790 | 2045 | 2240 | 2760 |

TABLE 54
Summary of Pharmacokinetic Parameters of TAK-935
Day 21
Part 2
<Pharmacokinetic Analysis Set>
Analyte: TAK-935

| Variable | Dose | N | Mean | SD | CV | Minimum | Q1 | Median | Q3 | Maximum |
|---|---|---|---|---|---|---|---|---|---|---|
| T1/2z (h) | TAK-935 | 6 | 2.927 | 1.3064 | 44.6 | 1.99 | 2.000 | 2.240 | 3.990 | 5.10 |
| Lambda z (1/h) | TAK-935 | 6 | 0.2708 | 0.092155 | 34.0 | 0.136 | 0.1740 | 0.3095 | 0.3470 | 0.349 |
| CL/F (L/H) | TAK-935 | 6 | 158.0 | 46.135 | 29.2 | 109 | 134.0 | 147.0 | 168.0 | 243 |
| Vz/F (L) | TAK-935 | 6 | 640.5 | 239.96 | 37.5 | 311 | 501.0 | 620.5 | 804.0 | 986 |

1. A method of treating a neurological disease in a human in need thereof, the method comprising administering to the human a therapeutically effective amount of a dosage form comprising soticlestat or a pharmaceutically acceptable salt thereof, wherein the human reaches a soticlestat steady-state plasma area under the curve (AUC₂₄) from about 449±219 to about 6118±4841 h×ng/ml following administration.

2. A method of treating a neurological disease in a human in need thereof, the method comprising administering to the human a therapeutically effective amount of a dosage form comprising soticlestat or a pharmaceutically acceptable salt thereof, wherein the human reaches a maximum blood plasma concentration (Cₘₐₓ) of from about 204±177 to about 2063±821 ng/ml following administration.

3. A method of treating a neurological disease in a human in need thereof, the method comprising administering to the human a therapeutically effective amount of soticlestat or a pharmaceutically acceptable salt thereof, wherein the human reaches a reduction from baseline in plasma 24HC of at least about 50% following administration.

4. A method of treating a neurological disease in a human in need thereof, the method comprising administering to the human a therapeutically effective amount of a solution comprising soticlestat or a pharmaceutically acceptable salt thereof, wherein the human reaches a soticlestat steady-state plasma area under the curve (AUC₂₄) from about 350 to about 2900 h×ng/ml following administration.

5. A method of treating a neurological disease in a human in need thereof, the method comprising administering to the human a therapeutically effective amount of a solution comprising soticlestat or a pharmaceutically acceptable salt thereof, wherein the human reaches a maximum blood plasma concentration (Cₘₐₓ) of from about 430 to about 2980 ng/ml.

6. A method of reducing plasma 24HC levels in a human, the method comprising administering to the human a therapeutically effective amount of soticlestat or a pharmaceutically acceptable salt thereof in a solution or a solid dosage form, wherein the human reaches a reduction from baseline in plasma 24HC of at least about 50% following administration.

7. A method of reducing seizure frequency in a human, the method comprising administering to the human a therapeutically effective amount of soticlestat or a pharmaceutically acceptable salt thereof in a solution or a solid dosage form, wherein the human achieves a reduction in seizure frequency of at least about 30% following administration.

8. The method according to any one of claims 1-7, wherein the therapeutically effective amount is a total daily dosage of from about 80 to about 600 mg of soticlestat or a pharmaceutically acceptable salt thereof.

9. The method according to claim 8, wherein the total daily dosage is about 200 mg, about 400 mg, or about 600 mg.

10. The method according to claim 8, wherein the total daily dose is 80 mg, 120 mg, 160 mg, 200 mg, 240 mg, or 400 mg for a human, wherein the human is a pediatric patient.

11. The method according to claim 9, wherein the total daily dosage is administered twice daily.

12. The method according to claim 11, wherein the human is a pediatric patient.

13. A method of minimizing the risk of one or more adverse events associated with soticlestat administration, the method comprising administering to a human having a neurological disease a total daily dosage of from about 80 mg to about 600 mg of soticlestat or a pharmaceutically acceptable salt thereof, wherein the soticlestat or a pharmaceutically acceptable salt thereof is administered in a solution or a solid dosage form.

14. The method according to any one of claim 1-10 or 12-13, wherein soticlestat or a pharmaceutically acceptable salt thereof is administered once daily.

15. The method according to any one of claims 1-13, wherein soticlestat or a pharmaceutically acceptable salt thereof is administered twice daily.

16. The method according to any one of claims 1-8, wherein the total daily dosage is about 100 mg once daily, about 200 mg once daily, about 300 mg once daily, or about 400 mg once daily.

17. The method according to any one of claims 1-8, wherein the total daily dosage is about 200 mg, about 400 mg, or about 600 mg, wherein the total daily dosage is administered in two administrations per day.

18. The method according to claim 1, wherein the human reaches an AUC₂₄ from about 800 to about 2290 h×ng/ml.

19. The method according to claim 4, wherein the human reaches an AUC₂₄ from about 800 to about 2900 h×ng/ml.

20. The method according to any one of claims 1-19, wherein the time to reach maximum blood plasma concentration (Tₘₐₓ) is about 2 hours or less after oral administration.

21. The method according to claim 20, wherein the time to reach maximum blood plasma concentration (Tₘₐₓ) is about 0.5 to 2 hours after oral administration.

22. The method according to any one of claims 1-21, wherein the terminal elimination half-life is from about 1.7 to about 7.1 hours.

23. The method according to any one of claims 1-22, wherein the total daily dosage is from about 200 to about 600 mg, wherein the human is administered soticlestat for at least 7 days, and wherein the AUC₁₂ on day 7 is within 160% of the AUC₁₂ on day 1.

24. The method according to any one of claims 1-23, wherein the total daily dosage is from about 200 to about 600 mg, wherein the human is administered soticlestat for at least 7 days, and wherein the Cₘₐₓ on day 7 is within 220% of the Cₘₐₓ on day 1.

25. The method of claim 1, wherein the therapeutically effective amount is a total daily dosage of about 200 mg, administered twice daily, and wherein the human reaches a soticlestat steady-state plasma area under the curve (AUC₂₄) of about 449 ng×h/ml±219 ng×h/ml.

26. The method of claim 1, wherein the therapeutically effective amount is a total daily dosage of about 400 mg, administered twice daily, and wherein the human reaches a soticlestat steady-state plasma area under the curve (AUC₂₄) of about 767 ng×h/ml±235 ng×h/ml.

27. The method of claim 1, wherein the therapeutically effective amount is a total daily dosage of about 600 mg, administered twice daily, and wherein the human reaches a soticlestat steady-state plasma area under the curve (AUC₂₄) of about 4036 ng×h/ml±1018 ng×h/ml.

28. The method of claim 2, wherein the therapeutically effective amount is a total daily dosage of about 200 mg, administered twice daily, and wherein the human reaches a maximum blood plasma concentration (Cₘₐₓ) of about 204 ng/ml±177 ng/ml.

29. The method of claim 2, wherein the therapeutically effective amount is a total daily dosage of about 400 mg, administered twice daily, and wherein the human reaches a maximum blood plasma concentration (Cₘₐₓ) of about 253 ng/ml±109 ng/ml.

30. The method of claim 2, wherein the therapeutically effective amount is a total daily dosage of about 600 mg, administered twice daily, and wherein the human reaches a maximum blood plasma concentration (Cₘₐₓ) of about 2063 ng/ml±831 ng/ml.

31. The method according to any one of claims 1-30, wherein the neurological disease is epilepsy, rare epilepsy, developmental epileptic encephalopathy, epileptic encephalopathy, Dravet syndrome, Lennox-Gastaut syndrome, focal onset seizures, major motor seizures, major motor drop seizures, focal seizures with secondary generalization, infantile spasms, primary generalized tonic-clonic seizures, partial onset seizures with our without secondary generalization, simple partial seizures, complex partial seizures, simple absence seizures, complex absence seizures, Dup15q syndrome, CDKL5 deficiency disorder, migraine, or complex regional pain syndrome.

32. The method according to any one of claims 1-30, wherein the neurological disease is developmental epileptic encephalopathy or epileptic type disease.

33. The method according to any one of claims 1-30, wherein the neurological disease is Dravet syndrome or Lennox-Gastaut syndrome.

34. The method according to any one of claims 1-33, wherein the soticlestat is a free base of crystalline Form II.

35. The method according to claim 34, wherein the crystalline Form II is characterized by (i) an x-ray powder diffraction (XRPD) pattern comprising three or more, four or more, five or more, six or more, seven or more, eight or more, nine or more, or ten 2θ values selected from 9.4, 10.8, 13.0, 15.3, 17.2, 18.2, 18.8, 19.4, 20.1, and 21.6±0.2°2θ, and/or (ii) an XPRD pattern substantially in accordance with FIG. 2.

36. The method according to any one of claims 1-33, wherein the soticlestat is a free base of crystalline Form I.

37. The method according to claim 36, wherein the crystalline Form I is characterized by (i) an x-ray powder diffraction (XRPD) pattern comprising three or more, four or more, five or more, six or more, seven or more, eight or more, nine or more, or ten 2θ values selected from 9.0, 9.6, 11.3, 12.3, 14.1, 15.7, 17.4, 20.9, 21.6, and 22.0±0.2°2θ, and/or (ii) an XPRD pattern substantially in accordance with FIG. 1.

38. The method according to any one of claims 1-33, wherein the soticlestat is a crystalline 3.0 hydrate of soticlestat.

39. The method according to claim 38, wherein the crystalline 3.0 hydrate is characterized by (i) an x-ray powder diffraction (XRPD) pattern comprising three or more, four or more, five or more, six or more, seven or more, eight or more, nine or more, or ten 2θ values selected from 8.8, 9.3, 12.4, 14.8, 16.9, 20.5, 20.9, 21.9, 22.3, and 24.5±0.2°2θ, and/or (ii) an XPRD pattern substantially in accordance with FIG. 3.

40. The method of claim 1, 2, 6, 7, or 13, wherein the solid dosage form is a tablet.

41. The method according to any one of claims 1-40, wherein soticlestat or a pharmaceutically acceptable salt thereof is administered daily for at least fourteen days, optionally wherein the human is in a fasted state at the time of administration.

42. A method of treating a neurological disease in a human in need thereof, the method comprising administering to the human about 100 mg once daily of soticlestat or a pharmaceutically acceptable salt thereof.

43. A method of treating a neurological disease in a human in need thereof, the method comprising administering to the human about 200 mg once daily of soticlestat or a pharmaceutically acceptable salt thereof.

44. A method of treating a neurological disease in a human in need thereof, the method comprising orally administering to the human about 300 mg once daily of soticlestat or a pharmaceutically acceptable salt thereof.

45. A method of treating a neurological disease in a human in need thereof, the method comprising administering to the human about 400 mg once daily of soticlestat or a pharmaceutically acceptable salt thereof.

46. A method of treating a neurological disease in a human in need thereof, the method comprising administering to the human a total daily dosage of about 200 mg of soticlestat or a pharmaceutically acceptable salt thereof, wherein the soticlestat or a pharmaceutically acceptable salt thereof is administered twice daily.

47. A method of treating a neurological disease in a human in need thereof, the method comprising administering to the human a total daily dosage of about 400 mg of soticlestat or a pharmaceutically acceptable salt thereof, wherein the soticlestat or a pharmaceutically acceptable salt thereof is administered twice daily.

48. A method of treating a neurological disease in a human in need thereof, the method comprising administering to the human a total daily dosage of about 600 mg of soticlestat or a pharmaceutically acceptable salt thereof, wherein the soticlestat or a pharmaceutically acceptable salt thereof is administered twice daily.

49. A method of treating a neurological disease in a human in need thereof, the method comprising administering to the human a total daily dosage of about 80 mg, 120 mg, 160 mg, 200 mg, 240 mg, or 400 mg of soticlestat or a pharmaceutically acceptable salt thereof, wherein the soticlestat or pharmaceutically acceptable salt thereof is administered twice daily.

50. The method according to claim 49, wherein the human is a pediatric patient.

51. The method of any of claims 42-50, wherein the soticlestat or pharmaceutically acceptable salt thereof is administered orally.

52. The method of any of claims 42-50, wherein the soticlestat or pharmaceutically acceptable salt thereof is administered by a gastrostomy or jejunostomy tube delivery.

53. The method of any of claims 42-52, wherein the soticlestat or pharmaceutically acceptable salt thereof is administered in an oral solution.

54. The method of any of claims 42-52, wherein the soticlestat or pharmaceutically acceptable salt thereof is administered in a solid oral dosage form.

55. The method of claim 54, wherein the solid oral dosage form is a tablet.

56. The method according to any one of claims 42-55, wherein the neurological disease is epilepsy, rare epilepsy, developmental epileptic encephalopathy, epileptic encephalopathy, Dravet syndrome, Lennox-Gastaut syndrome, focal onset seizures, major motor seizures, major motor drop seizures, focal seizures with secondary generalization, infantile spasms, primary generalized tonic-clonic seizures, partial onset seizures with our without secondary generalization, simple partial seizures, complex partial seizures, simple absence seizures, complex absence seizures, Dup15q syndrome, CDKL5 deficiency disorder, migraine, or complex regional pain syndrome.

57. The method according to any one of claims 42-55, wherein the neurological disease is developmental epileptic encephalopathy or epileptic type disease.

58. The method according to any one of claims 42-55, wherein the neurological disease is Dravet syndrome or Lennox-Gastaut syndrome.

59. The method according to any one of claims 42-58, wherein the soticlestat is a soticlestat free base of crystalline Form II.

60. The method according to claim 59, wherein the crystalline Form II is characterized by (i) an x-ray powder diffraction (XRPD) pattern comprising three or more, four or more, five or more, six or more, seven or more, eight or more, nine or more, or ten 2θ values selected from 9.4, 10.8, 13.0, 15.3, 17.2, 18.2, 18.8, 19.4, 20.1, and 21.6±0.2°2θ, and/or (ii) an XPRD pattern substantially in accordance with FIG. 2.

61. The method according to any one of claims 42-58, wherein the soticlestat is a soticlestat free base of crystalline Form I.

62. The method according to claim 61, wherein the crystalline Form I is characterized by (i) an x-ray powder diffraction (XRPD) pattern comprising three or more, four or more, five or more, six or more, seven or more, eight or more, nine or more, or ten 2θ values selected from 9.0, 9.6, 11.3, 12.3, 14.1, 15.7, 17.4, 20.9, 21.6, and 22.0±0.2°2θ, and/or (ii) an XPRD pattern substantially in accordance with FIG. 1.

63. The method according to any one of claims 42-58, wherein the soticlestat is a crystalline 3.0 hydrate of soticlestat.

64. The method according to claim 63, wherein the crystalline 3.0 hydrate is characterized by (i) an x-ray powder diffraction (XRPD) pattern comprising three or more, four or more, five or more, six or more, seven or more, eight or more, nine or more, or ten 2θ values selected from 8.8, 9.3, 12.4, 14.8, 16.9, 20.5, 20.9, 21.9, 22.3, and 24.5±0.2°2θ, and/or (ii) an XPRD pattern substantially in accordance with FIG. 3.

65. The method of any one of claims 1-13, wherein the dosage form is orally administered.

66. The method of any one of claims 1-13, wherein the dosage form is administered by gastronomy or jejunostomy tube.

67. The method of any preceding claim, wherein soticlestat is co-administered with an antiseizure medication.

68. The method of claim 67, wherein the co-administration is sequential.

69. The method of claim 67, wherein the co-administration is at the same time.

70. The method of any one of claims 67-69, wherein the antiseizure medication is valproic acid, clobazam, stiripentol, topiramate, clonazepam (klonipin), levetiracetam, zonisamide, gelbatolm, epidiolex, fintepla, zonisamide, pyridoxine, ethosuximide, diazepam, brivaracetam, prednisone, prednisolone methylprednisolone, fycompa, phenobarbital, valproate, or lamotrigine, rufinamide.
